(19) United States
(12) Patent Application Publication
(10) Pub. No.: US 2024/0117295 A1
(43) Pub. Date: Apr. 11, 2024

(54) MICROORGANISMS AND USES THEREOF

(71) Applicant: United Kingdom Research and Innovation, Swindon, Wiltshire (GB)

(72) Inventors: Wesley E. ROBERTSON, Swindon (GB); Louise F. H. FUNKE, Swindon (GB); Daniel DE LA TORRE, Swindon (GB); Julius FREDENS, Swindon (GB); Thomas S. ELLIOTT, Swindon (GB); Daniel L. DUNKELMANN, Swindon (GB); Julian C.W. WILLIS, Swindon (GB); Adam T. BEATTIE, Swindon (GB); Jason W. CHIN, Swindon (GB)

(21) Appl. No.: 18/288,340

(22) PCT Filed: May 28, 2021
(86) PCT No.: PCT/EP2021/064391

§ 371 Date: Oct. 25, 2023

(51) Int. Cl. C12N 1/20; C12N 9/00; C12N 15/10

(52) U.S. Cl. CPC C12N 1/20; C12N 9/93; C12N 15/1024; C12R 2001/19

(57) ABSTRACT

The invention relates to prokaryotic cells for the production of polymers containing non-canonical amino acids, and to methods for making said cells. The invention also relates to newly obtainable polymers as produced by the cells of the invention. In addition, the invention relates to new orthogonal aminoacyl-tRNA synthetases (aaRSs) and orthogonal tRNAs, which may be used in pairs and find utility in host cells such as, but not limited to, the prokaryotic cells of the invention.

### FIELD OF THE INVENTION

The present invention relates to novel prokaryotic cells for the production of polymers containing non-canonical amino acids, and to methods for making said cells. The invention also relates to newly obtainable polymers as produced by the prokaryotic cells of the invention. In addition, the invention relates to new orthogonal aminoacyl-tRNA synthetases (aaRSs) and orthogonal tRNAs, which may be used in pairs and find utility in host cells such as, but not limited to, the prokaryotic cells of the invention.

### BACKGROUND OF THE INVENTION

Nature uses 64 triplet codons to encode the synthesis of proteins composed of the twenty canonical amino acids, and most amino acids are encoded by more than one synonymous codon. It is widely hypothesized that removing sense codons and the tRNAs that read them from the genome may enable the creation of cells with several properties not found in natural biology—including new modes of viral resistance and the ability to encode the biosynthesis of non-canonical heteropolymers (3-6). However, these hypotheses have not been experimentally tested and so remain conjecture.

Removing release factor-1 (RF-1) (and therefore the ability to efficiently terminate translation on the TAG stop codon) from E. coli, provides some resistance to a limited subset of phage. However, this resistance is not general and phage are often propagated in the absence of RF-1 (8) because the TAG stop codon is rarely used for the termination of translation, and—even when viral genes do terminate in an amber codon—the inability to read a stop codon does not limit the synthesis of full-length viral proteins. In contrast, sense codons are commonly at least 10 times more abundant than amber codons in viral genomes, and occur over the length of viral genes.

Current strategies for encoding new monomers in cells are limited to encoding a single type of monomer (commonly in response to the amber stop codon) (3, 10, 11), inefficient, or incompatible with encoding sequential monomers (12-17); these limitations preclude the synthesis of non-canonical heteropolymer sequences composed entirely of non-canonical monomers.

Recently a strain of E. coli, Syn61, was created with a synthetic recoded genome in which all annotated occurrences of two sense codons (serine codons TCG and TCA) and a stop codon (TAG) were replaced with synonymous codons (18). This strain grows 1.6-fold slower than the strain from which it was derived.

A need, therefore, remains for new platforms for the synthesis of polymers containing non-canonical amino acids.

Some of the current platforms for the synthesis of polymers containing a non-canonical amino acid make use of an orthogonal aaRS/tRNA pair. Such pairs may be used to insert the non-canonical amino acid during protein synthesis. These pairs must be further engineered to decode a distinct target codon and to use a unique monomer that is not a substrate for other aaRSs.

Should a platform be developed that is capable of inserting multiple non-canonical amino acids into a polymer, it might need to make use of multiple aaRS/tRNA pairs. The identification of multiple engineered mutually orthogonal aaRS/tRNA pairs that recognise distinct codons and incorporate distinct non-canonical amino acids (ncAAs) remains an outstanding challenge. Each new ncAA, aaRS, tRNA and codon must function together and be orthogonal to each endogenous amino acid, aaRS and group of isoacceptor tRNAs and their cognate group of codons. Therefore, for each new ncAA:aaRS:tRNA:codon set, three interactions must be established (ncAA:aaRS, aaRS:tRNA and tRNA:codon) and 120 interactions (6×20 interactions; this analysis counts all isoacceptors for a natural amino acid as one and all codons for an amino acid as one and therefore provides a conservative estimate of the interactions that must be controlled) between the new set and the endogenous translational machinery must be minimised. Moreover, when incorporating more than one ncAA, there is the potential for interactions between components of the additional ncAA:aaRS:tRNA:codon sets, and these must also be minimised. Generating ncAA:aaRS:tRNA:codon sets to encode three distinct ncAAs into a polypeptide requires nine specific interactions to be established and minimization of at least 378 specific interactions, including 18 interactions between components of the three sets.

As such, a need exists for the provision of new orthogonal aaRS/tRNA pairs and for the identification of aaRS/tRNA pairs that would be functional if used in combination.

### SUMMARY OF THE INVENTION

Provided herein are prokaryotic cells that are suitable for the production of polymers containing non-canonical amino acids. The inventors demonstrate that it is possible to remove one, two, or more endogenous tRNAs from a prokaryotic cell, e.g. by deletion of the endogenous genes, to result in a viable cell. The inventors demonstrate that the removed endogenous tRNAs may be replaced with orthogonal tRNAs. The inventors further provide methods to overcome any growth defects that may be introduced by these processes. The inventors also provide experimental data showing that the modified prokaryotic cells are completely resistant to phage.

In an aspect of the invention, there is provided a prokaryotic cell, wherein: the prokaryotic cell does not express a first endogenous tRNA and a second endogenous tRNA; and the prokaryotic cell comprises a genome wherein a first type of sense codon and a second type of sense codon have been recoded such that the first endogenous tRNA and the second endogenous tRNA are dispensable.

In some embodiments, the essential genes of the genome do not contain occurrences of the first type of sense codon, and the first endogenous tRNA is a cognate tRNA for the first type of sense codon; and/or the essential genes of the genome do not contain occurrences of the second type of sense codon, and the second endogenous tRNA is a cognate tRNA for the second type of sense codon.

In some embodiments, the genome comprises 5, 4, 3, 2, 1, or no occurrences of the first type of sense codon, and the first endogenous tRNA is a cognate tRNA for the first type of sense codon; and/or the genome of comprises 5, 4, 3, 2, 1, or no occurrences of the second type of sense codon, and the second endogenous tRNA is a cognate tRNA for the second type of sense codon.

In some embodiments, the first type of sense codon is TCA and the first endogenous tRNA is tRNA^{Ser}_{UGA}; and/or the second type of sense codon is TCG and the second endogenous is tRNA^{Ser}_{CGA}. A plurality of occurrences of the TCA codon in the parental strain may have been replaced with AGT; and/or a plurality of occurrences of the TCG codon in the parental strain may have been replaced with AGC.

In some embodiments, the prokaryotic cell does not express a first endogenous release factor; and a first type of stop codon has been recoded within the genome such that the first endogenous release factor is dispensable.

The essential genes of the genome may not contain occurrences of the first type of stop codon, and the first endogenous release factor may be a cognate release factor for the first type of stop codon.

The genome may comprise 5, 4, 3, 2, 1, or no occurrences of the first type of stop codon, and the first endogenous release factor may be a cognate release factor for the first type of stop codon. The first type of stop codon may be TAG and wherein the first endogenous release factor may be RF-1. Occurrences of the TAG codon in the parental strain may have been replaced with TAA.

In some embodiments, the genome is at least 80%, 85%, 90%, 95%, 98%, 99%, 99.5%, 99.8%, 99.9%, or 100% identical to any one of SEQ ID NOs: 3 to 8.

In some embodiments, the prokaryotic cell expresses a first orthogonal aminoacyl-tRNA synthetase and a first orthogonal tRNA, the first orthogonal aminoacyl-tRNA synthetase and the first orthogonal tRNA form a first orthogonal aminoacyl-tRNA synthetase-tRNA pair, and the first orthogonal tRNA is capable of decoding the first type of sense codon.

The first orthogonal aminoacyl-tRNA synthetase may be MmPylRS or a variant with altered selectivity to a non-canonical amino acid, and the first orthogonal tRNA may be MmtRNA^{Pyl}_{YYY} or MmtRNA^{Pyl}_{UGA}.

In some embodiments, the prokaryotic cell expresses a second orthogonal aminoacyl-tRNA synthetase and a second tRNA, the second orthogonal aminoacyl-tRNA synthetase and the second orthogonal tRNA form a second orthogonal aminoacyl-tRNA synthetase-tRNA pair, and the second orthogonal tRNA is capable of decoding the second type of sense codon.

The second orthogonal aminoacyl-tRNA synthetase may be 1R26PylRS or a variant with altered selectivity to a non-canonical amino acid, and the second orthogonal tRNA may be AlvtRNA^{ΔNPyl(8)}_{YYY} or AlvtRNA^{ΔNPyl(8)}_{CGA}.

In some embodiments, the prokaryotic cell expresses a third orthogonal aminoacyl-tRNA synthetase and a third orthogonal tRNA, the third orthogonal aminoacyl-tRNA synthetase and the third orthogonal tRNA form a third orthogonal aminoacyl-tRNA synthetase-tRNA pair, and the third orthogonal tRNA is capable of decoding the first type of stop codon.

The third orthogonal aminoacyl-tRNA synthetase-tRNA pair may be AfTyrRS or a variant with altered selectivity to a non-canonical amino acid, and Af-tRNA^{Tyr(A01)}_{YYY}; or MjTyrRS or a variant with altered selectivity to a non-canonical amino acid, and MjtRNA^{Tyr}_{YYY}.

The growth rate of the prokaryotic cell may be faster than the growth rate of a reference prokaryotic cell of a parental strain. The reference prokaryotic cell may be of a parental strain directly obtained upon recoding of the genome to remove the first type of sense codon, the second type of sense codon, and the first type of stop codon. The reference prokaryotic cell may be of a parental strain directly obtained upon removal of the first endogenous tRNA, the second endogenous tRNA, and the first endogenous release factor.

The prokaryotic cell may be resistant to phage infection and/or horizontal transfer of the F plasmid. The prokaryotic cell may be completely resistant to phage infection and/or horizontal transfer of the F plasmid.

The prokaryotic cell may be a bacterial cell or an Escherichia coli cell. The prokaryotic cell is viable.

In an aspect of the invention, there is provided a prokaryotic cell with a genome that is at least 80%, 85%, 90%, 95%, 98%, 99%, 99.5%, 99.8%, 99.9%, or 100% identical to any one of SEQ ID NOs: 3 to 8. The calculation of the sequence identity may exclude any exogenous sequences, for instance any sequences for the insertion of orthogonal aaRS/tRNA pairs.

In another aspect of the invention, there is provided a method for producing a modified prokaryotic cell, wherein the method comprises:
(i) modifying a prokaryotic cell to express a first orthogonal aminoacyl-tRNA synthetase-tRNA pair suitable for decoding a first type of sense codon, wherein
   the prokaryotic cell comprises a genome wherein the first type of sense codon has been recoded such that a first endogenous tRNA is dispensable;
(ii) incubating the prokaryotic cell in the presence of a non-canonical amino acid which is a substrate for the first orthogonal aminoacyl-tRNA synthetase; and
(iii) modifying the endogenous gene encoding the first endogenous tRNA such that the first endogenous tRNA is not expressed.

In an embodiment, steps (i) and (ii) are performed before step (iii). In another embodiment, step (iii) is performed before step (i) and (ii).

The method may further comprise:
(a) modifying the prokaryotic cell to express a second orthogonal aminoacyl-tRNA synthetase-tRNA pair suitable for decoding a second type of sense codon, wherein
   the prokaryotic cell comprises a genome wherein the second type of sense codon has been recoded such that a second endogenous tRNA is dispensable;
(b) incubating the prokaryotic cell in the presence of a non-canonical amino acid which is a substrate for the second orthogonal aminoacyl-tRNA synthetase; and
(c) modifying the endogenous gene encoding the second endogenous tRNA such that the second endogenous tRNA is not expressed.

In an embodiment, steps (i) and (ii) are performed before step (iii), and/or steps (a) and (b) are performed before step (c). In another embodiment, step (iii) is performed before steps (i) and (ii), and/or step (c) is performed before steps (a) and (b).

In some embodiments, steps (a), (b), and (c) are performed before steps (i), (ii), (iii); or step (a) is performed concurrently with step (i), step (b) is performed concurrently with step (ii), and step (c) is performed concurrently with step (iii); or steps (a), (b), and (c) are performed after steps (i), (ii), (iii).

The essential genes of the genome may not contain occurrences of the first type of sense codon, or the genome may comprise 5, 4, 3, 2, 1, or no occurrences of the first type of sense codon. The first type of sense codon may be TCA and the first endogenous tRNA may be tRNA^{Ser}_{UGA}. The TCA codon may have been replaced with AGT.

The first orthogonal aminoacyl-tRNA synthetase may be MmPylRS or a variant with altered selectivity to a non-canonical amino acid, and the first orthogonal tRNA may be MmtRNA^{Pyl}_{YYY} or MmtRNA^{Pyl}_{UGA}.

The essential genes of the genome may not contain occurrences of the second type of sense codon, or the genome of may comprise 5, 4, 3, 2, 1, or no occurrences of the second type of sense codon. The second type of sense codon may be TCG and the second endogenous tRNA may be tRNA^{Ser}_{CGA}. The TCG codon may be replaced with AGC.

The second orthogonal aminoacyl-tRNA synthetase may be 1R26PylRS or a variant with altered selectivity to a non-canonical amino acid, and the second orthogonal tRNA may be AlvtRNA^{ΔNPyl(8)}_{YYY} or AlvtRNA^{ΔNPyl(8)}_{CGA}.

The genome of the prokaryotic cell to which the method is applied may be at least 80%, 85%, 90%, 95%, 98%, 99%, 99.5%, 99.8%, 99.9%, or 100% identical to any one of: GenBank accession number CP040347.1, SEQ ID NO: 3, or SEQ ID NO: 4.

The method may further comprise modifying the prokaryotic cell to express a third orthogonal aminoacyl-tRNA synthetase-tRNA pair suitable for decoding a first type of stop codon, wherein a first type of stop codon has been recoded within the genome such that a first endogenous release factor is dispensable. The essential genes of the genome may not contain occurrences of the first type of stop codon or the genome may comprise 5, 4, 3, 2, 1, or no occurrences of the first type of stop codon. The first type of stop codon may be TAG and wherein the cognate release factor for the first type of stop codon may be RF-1. The TAG codon may be replaced with TAA.

The third orthogonal aminoacyl-tRNA synthetase-tRNA pair may be AfTyrRS or a variant with altered selectivity to a non-canonical amino acid, and Af-tRNA^{Tyr(A01)}_{YYY}; or MjTyrRS or a variant with altered selectivity to a non-canonical amino acid, and MjtRNA^{Tyr}_{YYY}.

The method may comprise, prior to step (i) and (a):
inducing mutagenesis in a cell culture comprising the prokaryotic cell that comprises the recoded genome,
maintaining the cell culture under exponential growth conditions,
selecting a prokaryotic cell, from said cell culture, with an increased growth rate compared to the initial culture; and
   wherein step (i) or (a) may be applied to the selected prokaryotic cell.

The mutagenesis, mutation, and selection may be part of a parallel mutagenesis and dynamic parallel selection process. The induction of mutagenesis may comprise the use of a mutagenesis plasmid. The mutagenesis plasmid may be MP6.

In an embodiment, prior to step (i), two rounds of mutagenesis and selection are applied.

The method may further comprises, after step (iii) and/or (c):
obtaining a cell culture from the prokaryotic cell,
inducing mutagenesis in the cell culture,
maintaining the cell culture under exponential growth conditions, and
selecting a prokaryotic cell, from said cell culture, with an increased growth rate compared to the initial culture.

The mutagenesis, mutation, and selection may be part of a parallel mutagenesis and dynamic parallel selection process. The induction of mutagenesis may comprise the use of a mutagenesis plasmid, wherein the mutagenesis plasmid does not contain any occurrences of the first or second type of sense codon nor any occurrences of the first type of stop codon. The mutagenesis plasmid may be MP6, wherein the MP6 has been recoded to not contain any occurrences of the first or second type of sense codon nor any occurrences of the first type of stop codon. Three rounds of mutagenesis and selection may be applied.

In methods of the invention, mutagenesis may be carried out for 5, 10, 15, 17, 20, 30, 45, 60, 70, 80, 100, 150, 200, or more generations; and/or the cell culture may be maintained under exponential growth conditions for 5, 10, 15, 20, 30, 40, 50, 52, 60, 70, 80, 100, 200, or more generations.

In some embodiments, the prokaryotic cell is a bacterial cell or an Escherichia coli cell.

In an aspect of the invention, there is provided a method of synthesising a polymer, comprising:
i) providing a prokaryotic cell of the invention, or generated according to the methods of the invention, wherein the prokaryotic cell comprises the first orthogonal aminoacyl-tRNA synthetase-tRNA pair; and
contacting said prokaryotic cell with a nucleic acid sequence encoding a polymer, said nucleic acid sequence comprising the first type of sense codon;
ii) incubating the prokaryotic cell in the presence of a first non-canonical amino acid, wherein the first non-canonical amino acid is a substrate for the first orthogonal aminoacyl-tRNA synthetase; and
iii) incubating the prokaryotic cell to allow incorporation of the first non-canonical amino acid into the polymer via the first orthogonal aminoacyl-tRNA synthetase-tRNA pair.

In an embodiment, the prokaryotic cell comprises the second orthogonal aminoacyl-tRNA synthetase-tRNA pair and the nucleic acid sequence comprises the second type of sense codon, and
step ii) comprises incubating the prokaryotic cell in the presence of a second non-canonical amino acid, wherein the second non-canonical amino acid is a substrate for the second orthogonal aminoacyl-tRNA synthetase; and
step iii) comprises incubating the prokaryotic cell to allow incorporation of the second non-canonical amino acid into the polymer via the second first orthogonal aminoacyl-tRNA synthetase-tRNA pair.

In another embodiment, the prokaryotic cell comprises the third orthogonal aminoacyl-tRNA synthetase-tRNA pair and the nucleic acid sequence comprises the first type of stop codon, and
step ii) comprises incubating the prokaryotic cell in the presence of a third non-canonical amino acid, wherein the third non-canonical amino acid is a substrate for the third orthogonal aminoacyl-tRNA synthetase; and
step iii) comprises incubating the prokaryotic cell to allow incorporation of the third non-canonical amino acid into the polymer via the third first orthogonal aminoacyl-tRNA synthetase-tRNA pair.

Any of the methods of synthesising a polymer, may further comprise purifying the synthesised polymer.

The synthesised polymer may comprise at least one non-canonical amino acid directly adjacent to another non-canonical amino acid. The polymer may comprises a chain of two, three, four, five, six, seven, eight, nine, ten, 15, 20, or more non-canonical amino acids directly adjacent to each other. The polymer may be a macrocycle. The non-canonical amino acids may be any one of, or any combination of, BocK, CbzK, AllocK, p-I-Phe, CypK, AlkK, 3-Nitro-Tyr, and p-Az-Phe.

In an aspect of the invention, there is provided a polymer obtained from or obtainable by the methods of synthesising a polymer of the invention.

In an aspect of the invention, there is provided an isolated 1R26PylRS, or a variant with altered selectivity to a non-canonical amino acid. The isolated 1R26PylRS may be at least 50%, 60%, 70%, 80%, 90%, 95%, 99%, or 100% identical to SEQ ID NO: 9, comprising any one of the following groups of mutations: i) L121L, L125L, Y126G, M129L, N166N, V168V, Y206Y, A223A; ii) L121L, L125L, Y126Y, M129A, N166N, V168V, Y206F, A223A; iii) L121L, L125L, Y126Y, M129M, N166Q, V168V, Y206F, A223A; iv) L121L, L125L, Y126Y, M129M, N166S, V168M, Y206F, A223G; and v) L121M, L125I, Y126F, M129A, N166N, V168F, Y206Y, A223A. The isolated 1R26PylRS may be at least 50%, 60%, 70%, 80%, 90%, 95%, 99%, or 100% similar or identical to any one of SEQ ID NOs: 10 to 14.

In an aspect of the invention, there is provided an isolated Archaeoglobus fulgidus tyrosyl-tRNA synthetase (AfTyrRS), or a variant with altered selectivity to a non-canonical amino acid. The isolated AfTyrRS may be at least 50%, 60%, 70%, 80%, 90%, 95%, 99%, or 100% identical to SEQ ID NO: 16, comprising any one of the following groups of mutations: i) Y36I, L69M, H74L, Q116E, D165T, I166G, and N190N; ii) Y36T, L69L, H74L, Q116E, D165T, I166G, N190K; and iii) Y36I, L69L, H74L, Q116E, D165T, I166G, N190N. The isolated AfTyrRS may be at least 50%, 60%, 70%, 80%, 90%, 95%, 99%, or 100% similar or identical to any one of SEQ ID NOs: 16 to 19.

In an aspect of the invention, there is provided an isolated Lum1PylRS, or a variant with altered selectivity to a non-canonical amino acid. The Lum1PylRS may be at least 50%, 60%, 70%, 80%, 90%, 95%, 99%, or 100% identical to SEQ ID NO: 32, comprising any one of the following groups of mutations: i) L121L, L125L, Y126G, M129L, V168V; and ii) L121M, L125I, Y126F, M129A, V168F. The Lum1PylRS may be at least 50%, 60%, 70%, 80%, 90%, 95%, 99%, or 100% similar or identical to any one of SEQ ID NOs: 32 to 34.

In an aspect of the invention, there is provided an isolated NitroPylRS, or a variant with altered selectivity to a non-canonical amino acid. The NitroPylRS may be at least 50%, 60%, 70%, 80%, 90%, 95%, 99%, or 100% identical to SEQ ID NO: 36, comprising the following substitutions L123M, L127I, Y128F, M131A, and V169F. The NitroPylRS may be at least 50%, 60%, 70%, 80%, 90%, 95%, 99%, or 100% similar or identical to SEQ ID NO: 35 or SEQ ID NO: 36.

In an aspect of the invention, there is provided an isolated ClosΔNTDPylRS, or a variant with altered selectivity to a non-canonical amino acid. The ClosΔNTDPylRS may be at least 50%, 60%, 70%, 80%, 90%, 95%, 99%, or 100% identical to SEQ ID NO: 37, comprising any one of the following groups of mutations: i) Y126G, M129L, Y208Y, and ii) Y126Y, M129A, Y208F. The ClosΔTDPylRS may be at least 50%, 60%, 70%, 80%, 90%, 95%, 99%, or 100% similar or identical to any one of SEQ ID NOs: 37 to 39.

In an aspect of the invention, there is provided an isolated TronΔNTDPylRS, or a variant with altered selectivity to a non-canonical amino acid. The TronΔNTDPylRS may be at least 50%, 60%, 70%, 80%, 90%, 95%, 99%, or 100% similar or identical to SEQ ID NO: 40.

In an aspect of the invention, there is provided an isolated GemmΔNTDPylRS, or a variant with altered selectivity to a non-canonical amino acid. The GemmΔNTDPylRS may be at least 50%, 60%, 70%, 80%, 90%, 95%, 99%, or 100% similar or identical to SEQ ID NO: 41.

In an aspect of the invention, there is provided an isolated PGA8ΔNTDPylRS, or a variant with altered selectivity to a non-canonical amino acid. The PGA8ΔNTDPylRS may be at least 50%, 60%, 70%, 80%, 90%, 95%, 99%, or 100% similar or identical to SEQ ID NO: 42.

In an aspect of the invention, there is provided an isolated I2ΔNTDPylRS, or a variant with altered selectivity to a non-canonical amino acid. The I2ΔNTDPylRS may be at least 50%, 60%, 70%, 80%, 90%, 95%, 99%, or 100% similar or identical to SEQ ID NO: 43.

In an aspect of the invention, there is provided an isolated D121ΔNTDPylRS, or a variant with altered selectivity to a non-canonical amino acid. The D121ΔNTDPylRS may be at least 50%, 60%, 70%, 80%, 90%, 95%, 99%, or 100% similar or identical to SEQ ID NO: 44.

In an aspect of the invention, there is provided an isolated D416ΔNTDPylRS, or a variant with altered selectivity to a non-canonical amino acid. The D416ΔNTDPylRS may be at least 50%, 60%, 70%, 80%, 90%, 95%, 99%, or 100% similar or identical to SEQ ID NO: 45.

The tRNA synthetase of the invention may have altered selectivity towards any of: AllocK, AlkK, BocK, Bta, CbzK, CypK, 3-Nitro-Tyr, NMH, p-I-Phe, and p-Az-Phe, and o-Methyl-Tyrosine.

In an aspect of the invention, there is provided an isolated tRNA, wherein the isolated tRNA is at least 80%, 90%, 95%, 99%, or 100% identical to any one of SEQ ID NOs: 15, 20, 46-52, 54-58, 61-66.

In an aspect of the invention, there is provided a host cell comprising a tRNA synthetase according to the invention and a tRNA, wherein the tRNA synthetase and tRNA form an orthogonal aminoacyl-tRNA synthetase-tRNA pair.

In an aspect of the invention, there is provided a host cell comprising any combination of two or three items selected from the list:
(i) the tRNA synthetase according to the invention and a tRNA, wherein the tRNA synthetase and tRNA form an orthogonal aminoacyl-tRNA synthetase-tRNA pair;
(ii) an Archaeoglobus fulgidus tyrosyl-tRNA synthetase (AfTyrRS), or a variant with altered selectivity to a non-canonical amino acid, and an Af-tRNA^{Tyr(A01)}_{YYY}, wherein the AfTyrRS and Af-tRNA^{Tyr(A01)}_{YYY} form an orthogonal aminoacyl-tRNA synthetase-tRNA pair;
(iii) a MmPylRS tRNA synthetase, or a variant with altered selectivity to a non-canonical amino acid, and a MmtRNA^{Pyl}_{YYY}, wherein the MmPylRS tRNA synthetase and the MmtRNA^{Pyl}_{YYY} form an orthogonal aminoacyl-tRNA synthetase-tRNA pair.

In an aspect of the invention, there is provided a host cell comprising any combination of two or three items selected from the list:
(i) the tRNA synthetase according to the invention and a tRNA, wherein the tRNA synthetase and tRNA form an orthogonal aminoacyl-tRNA synthetase-tRNA pair;
(ii) an AfTyrRS, or a variant with altered selectivity to a non-canonical amino acid, and an Af-tRNA^{Tyr(A01)}_{YYY}, wherein the AfTyrRS and Af-tRNA^{Tyr(A01)}_{YYY} form an orthogonal aminoacyl-tRNA synthetase-tRNA pair;
(iii) an M jannaschii tyrosyl-tRNA synthetase (MjTyrRS), or a variant with altered selectivity to a non-canonical amino acid, and a MjtRNA^{Tyr}_{YYY}, wherein the MjTyrRS and the MjtRNA^{Tyr}_{YYY} form an orthogonal aminoacyl-tRNA synthetase-tRNA pair.

In an aspect of the invention, there is provided a host cell comprising any combination of two or three items selected from the list: i) a class A ΔNPylRS/^{ΔNPyl}tRNA pair; ii) a class B ΔNPylRS/^{ΔNPyl}tRNA pair; and iii) an MmPylRS/Spe^{Pyl}tRNA pair.

The host cell may be a prokaryotic cell or eukaryotic cell, a bacterial cell, an E. coli. cell, a mammalian cell, an insect cell, or a human cell.

In an aspect of the invention, there is provided a method for improving the growth rate of a prokaryotic cell wherein the genome has been recoded, wherein the method comprises:
inducing mutagenesis in a cell culture comprising the prokaryotic cell that comprises the recoded genome,
maintaining the cell culture under exponential growth conditions,
selecting a prokaryotic cell, from said cell culture, with an increased growth rate compared to the initial culture.

The mutagenesis, mutation, and selection may be part of a parallel mutagenesis and dynamic parallel selection process. The induction of mutagenesis may comprise the use of a mutagenesis plasmid. The mutagenesis plasmid may be a recoded mutagenesis plasmid, for instance recoded to remove a first and/or a second type of sense codon, and optionally a first type of stop codon. The mutagenesis plasmid may be MP6. Two rounds of mutagenesis and selection may be applied. The mutagenesis may be carried out for 5, 10, 15, 17, 20, 30, 45, 60, 70, 80, 100, 150, 200, or more generations; and/or the cell culture may be maintained under exponential growth conditions for 5, 10, 15, 20, 30, 40, 50, 52, 60, 70, 80, 100, 200, or more generations. The prokaryotic cell may be a bacterial cell or an Escherichia coli cell.

In an aspect of the invention, there is provided a prokaryotic cell obtained or obtainable by any of the methods for improving the growth rate of a prokaryotic cell of the invention, wherein the growth rate of the prokaryotic cell is faster than the growth rate of a reference prokaryotic cell of a parental strain.

In an aspect of the invention, there is provided a prokaryotic cell, wherein the prokaryotic cell: does not express a first endogenous tRNA; comprises a genome wherein a first type of sense codon has been recoded such that the first endogenous tRNA is dispensable; expresses a first orthogonal aminoacyl-tRNA synthetase and a first orthogonal tRNA, the first orthogonal aminoacyl-tRNA synthetase and the first orthogonal tRNA form a first orthogonal aminoacyl-tRNA synthetase-tRNA pair, and the first orthogonal tRNA is capable of decoding the first type of sense codon.

The essential genes of the genome may not contain occurrences of the first type of sense codon, and the first endogenous tRNA may be a cognate tRNA for the first type of sense codon. The genome may comprise 5, 4, 3, 2, 1, or no occurrences of the first type of sense codon, and the first endogenous tRNA may be a cognate tRNA for the first type of sense codon. The first type of sense codon may be TCA and the first endogenous tRNA may be tRNA^{Ser}_{UGA}; or the first type of sense codon may be TCG and the first endogenous may be tRNA^{Ser}_{CGA}. A plurality of occurrences of the TCA codon in the parental strain may have been replaced with AGT; and/or a plurality of occurrences of the TCG codon in the parental strain may have been replaced with AGC.

In a further embodiment, the prokaryotic cell does not express a second endogenous tRNA; comprises a genome wherein a second type of sense codon has been recoded such that the second endogenous tRNA is dispensable; expresses a second orthogonal aminoacyl-tRNA synthetase and a second tRNA, the second orthogonal aminoacyl-tRNA synthetase and the 20 second orthogonal tRNA form a second orthogonal aminoacyl-tRNA synthetase-tRNA pair; and the second orthogonal tRNA is capable of decoding the second type of sense codon. The essential genes of the genome may not contain occurrences of the second type of sense codon, and the second endogenous tRNA may be a cognate tRNA for the second type of sense codon. The genome may comprise 5, 4, 3, 2, 1, or no occurrences of the second type of sense codon, and the second endogenous tRNA may be a cognate tRNA for the second type of sense codon. The second type of sense codon may be TCA and the second endogenous tRNA may be tRNA^{Ser}_{UGA}; or the second type of sense codon may be TCG and the second endogenous may be tRNA^{Ser}_{CGA}.

In a further embodiment, the prokaryotic cell does not express a first endogenous release factor; a first type of stop codon has been recoded within the genome such that the first endogenous release factor is dispensable; the prokaryotic cell expresses a third orthogonal aminoacyl-tRNA synthetase and a third orthogonal tRNA; the third orthogonal aminoacyl-tRNA synthetase and the third orthogonal tRNA form a third orthogonal aminoacyl-tRNA synthetase-tRNA pair; and the third orthogonal tRNA is capable of decoding the first type of stop codon. The essential genes of the genome may not contain occurrences of the first type of stop codon, and the first endogenous release factor may be a cognate release factor for the first type of stop codon. The genome may comprise 5, 4, 3, 2, 1, or no occurrences of the first type of stop codon, and the first endogenous release factor may be a cognate release factor for the first type of stop codon. The first type of stop codon may be TAG and wherein the first endogenous release factor may be RF-1. Occurrences of the TAG codon in the parental strain may have been replaced with TAA.

The prokaryotic cell may be a bacterial cell or an Escherichia coli cell. The prokaryotic cell is viable.

### BRIEF DESCRIPTION OF THE DRAWINGS

FIG. 1. Strain evolution and creation of Syn61Δ3.

(A) Schematic of strain evolution. The codons that encode serine and protein termination are connected to the anticodons of the tRNAs or release factors predicted to decode them by black lines. The genes encoding the corresponding tRNAs and release factors are indicated in the black boxes. Cells with the decoding rules of Syn61 are denoted with a pink box throughout. Two rounds of parallel mutagenesis and dynamic selection created Syn61, Syn61(ev2). serT, serU, and prfA were then deleted to create Syn61Δ3. Finally, three rounds of parallel mutagenesis and dynamic selection were applied to create Syn61Δ3(ev5); Syn61Δ3 and Syn61Δ3(ev5) are represented by the light teal box throughout.

(B) Growth rates of Syn61 and all intermediate strains in the development of Syn61Δ3(ev5). Growth rates were calculated based on growth curves measured for n=8 replicate cultures for each strain. For statistics see Methods.

FIG. 2. Lytic phage propagation and cell lysis is obstructed in Syn61Δ3.

(A) Schematic of viral infection of Syn61Δ3. Deletion of serU (encoding tRNA^{Ser}_{CGA}), serT (encoding tRNA^{Ser}_{UGA}), and prfA (encoding RF-1) makes the UCG, UCA and UAG codons unreadable and the ribosome will stall at these codons within an mRNA that contains them, as shown here for a viral mRNA.

(B) Schematic of the number of TCG, TCA, and TAG codons and their positions in the genome of T6 phage.

(C) Cultures were infected with T6 phage at a multiplicity of infection (MOI) of 5·10⁻² and the total titer (intracellular phage plus free phage) was monitored over 4 hours. Treatment with gentamicin was used to ablate protein synthesis and provides a control for cells that cannot synthesize viral proteins or produce new viral particles.

(D) T6 efficiently lyses Syn61 variants but not Syn61Δ3. Cultures were infected as in panel (C) and OD₆₀₀ was measured after 4 hours.

(E) Number of the indicated codons per kb in each indicated phage.

(F-G) Syn61Δ3 survives simultaneous infection of multiple phage: (F) photos of the culture at the indicated timepoints following infection (+) or without infection (−). Cultures were infected with phage λ, P1, T4, T6, and T7, each with MOI=1·10⁻². (G) OD₆₀₀ of the cultures was measured after 4 hours. All experiments were performed in three independent replicates, the dots represent the independent replicates and the line (panel (C)) or bar (panels (D) and (G)) represents the mean. The photo (panel (F)) is a representative of data from three independent replicates.

FIG. 3. Reassigning two sense codons and a stop codon to non-canonical amino acid (ncAAs) in Syn61Δ3.

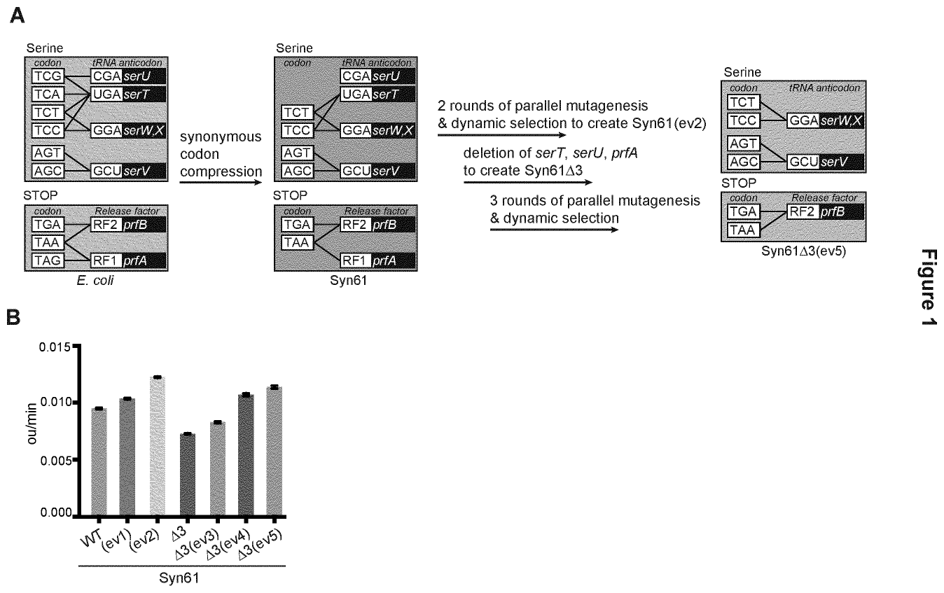

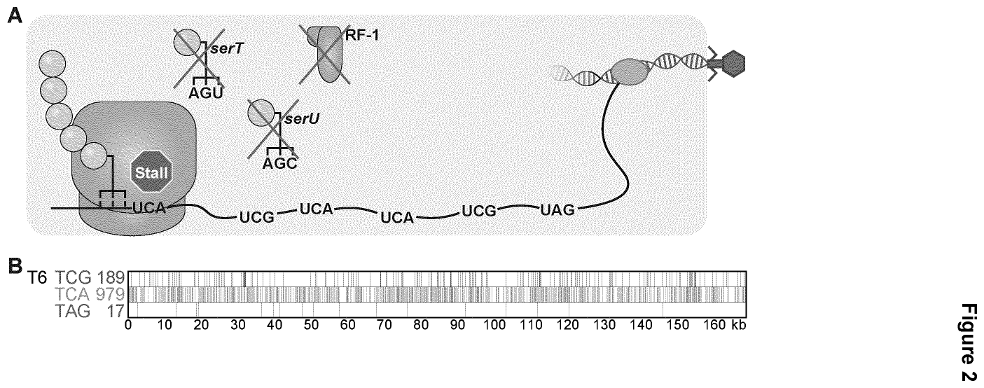

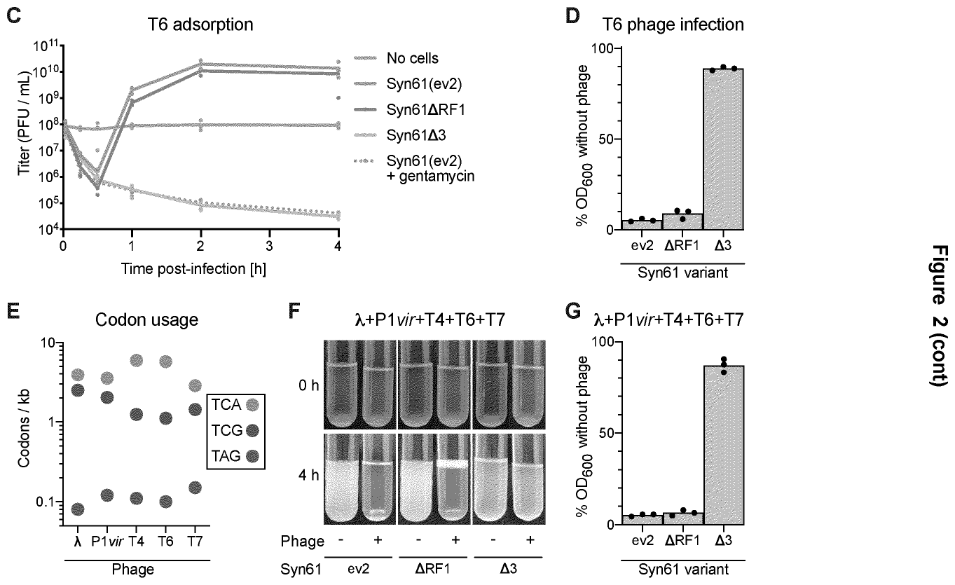

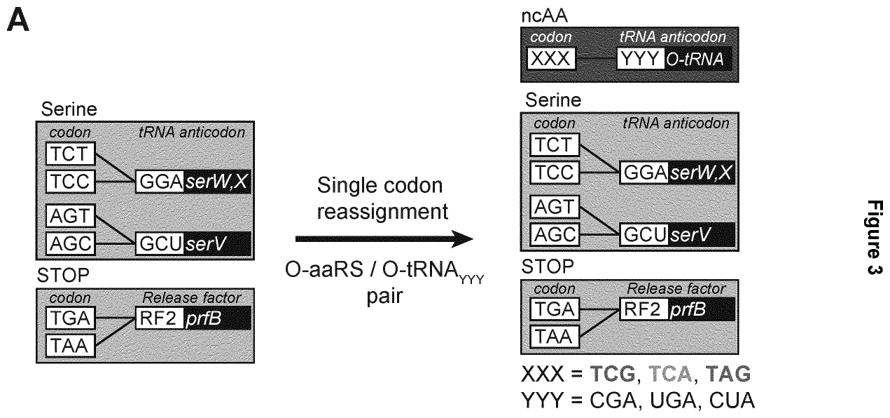

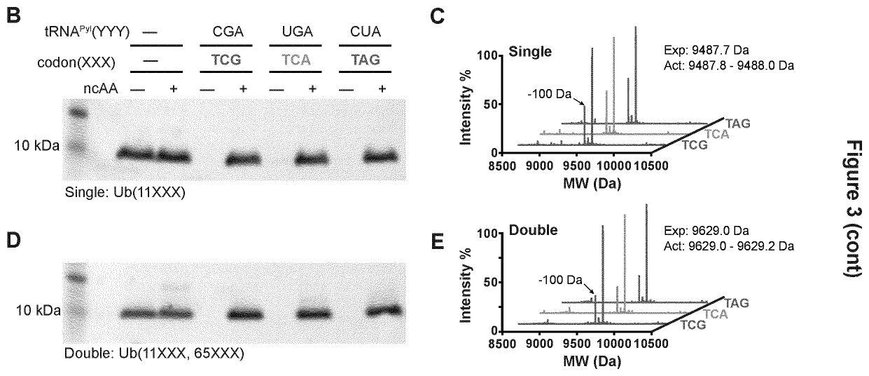

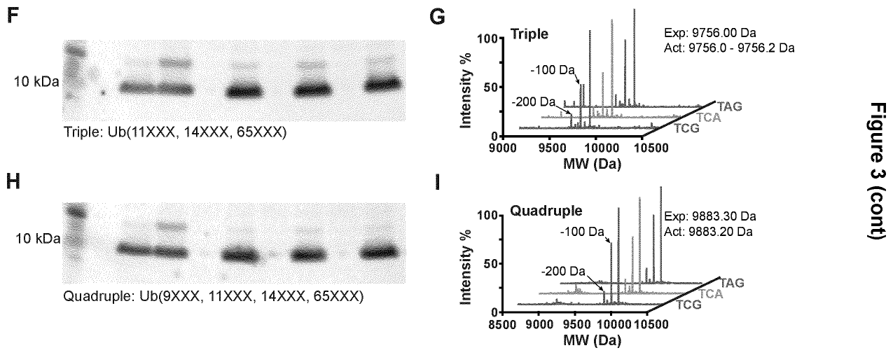

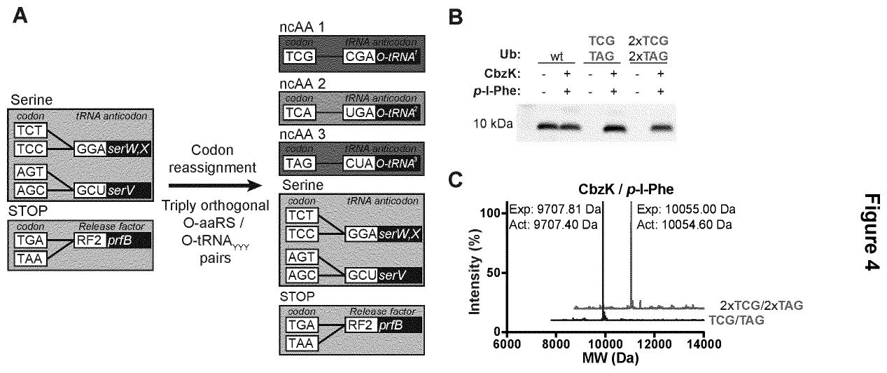

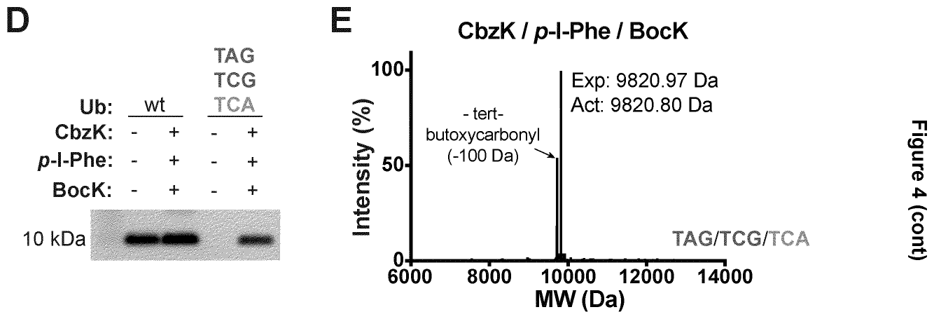

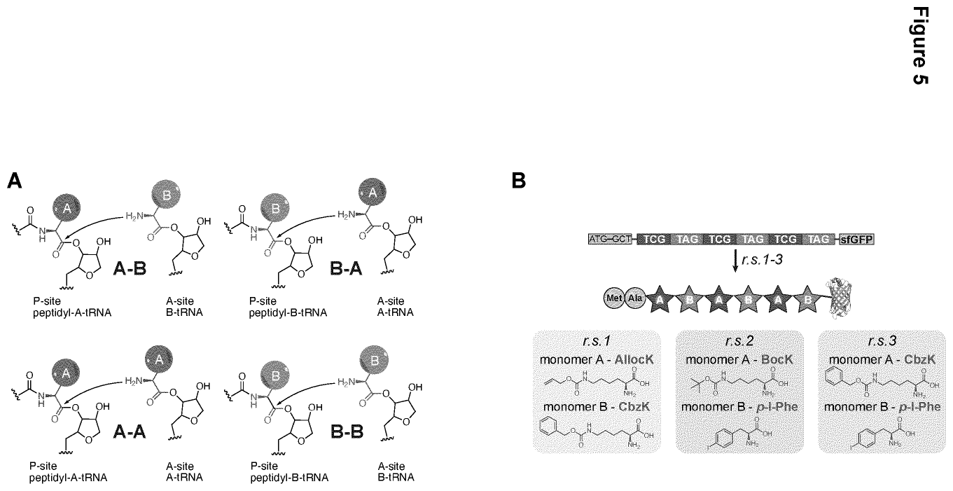

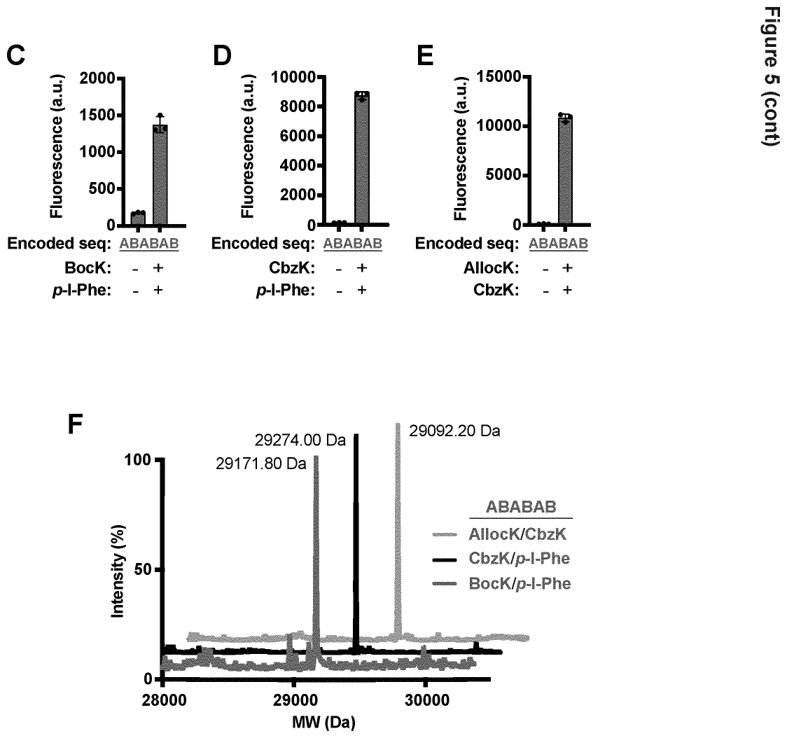

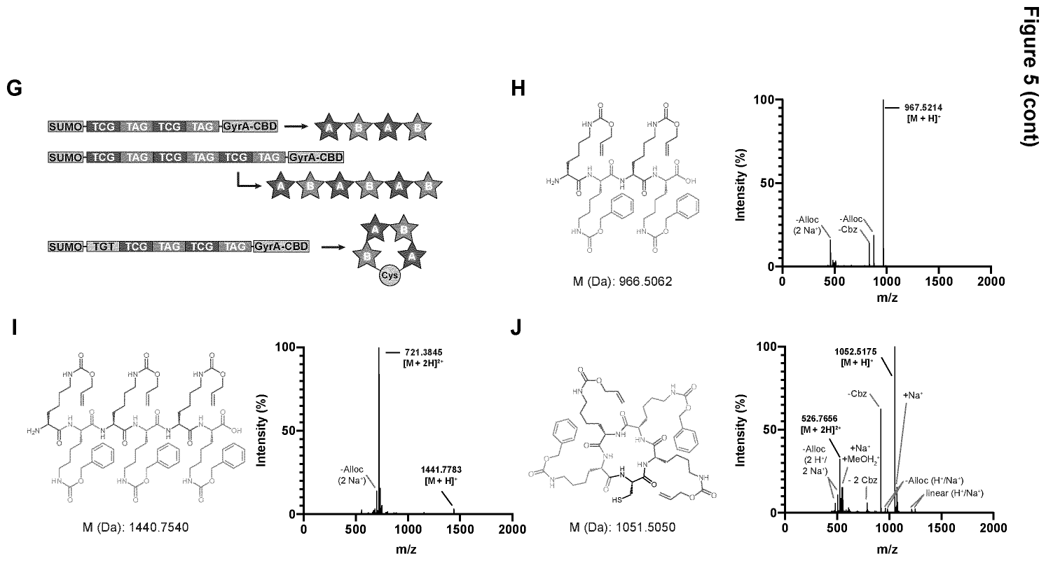

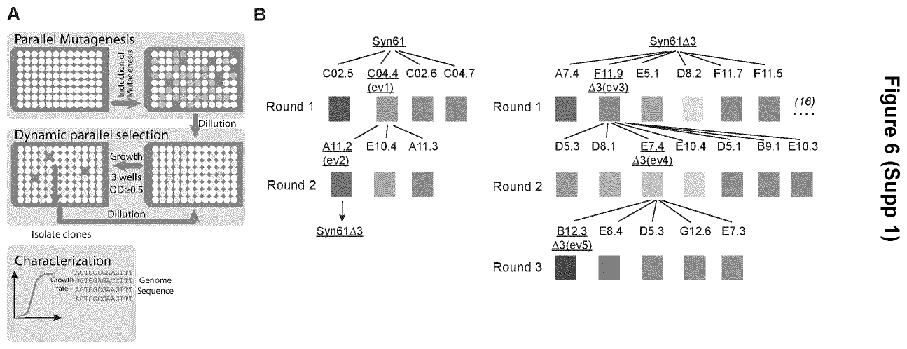

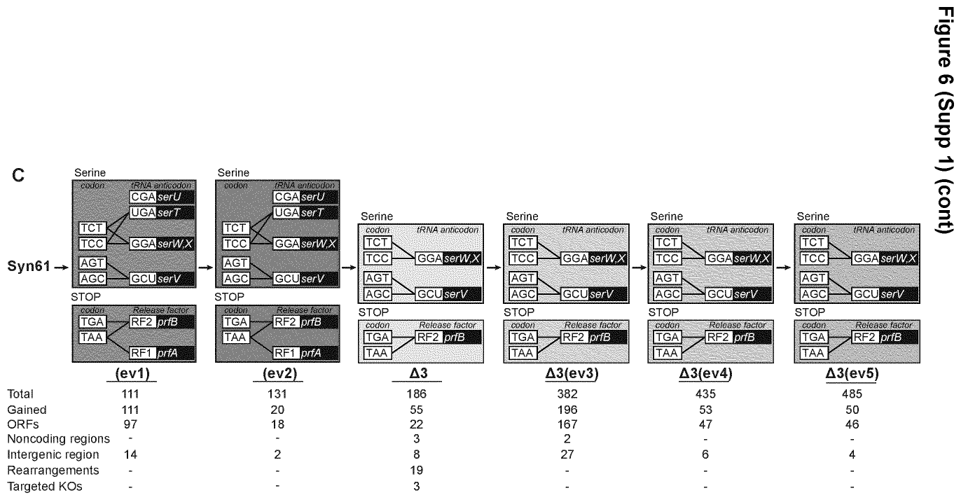

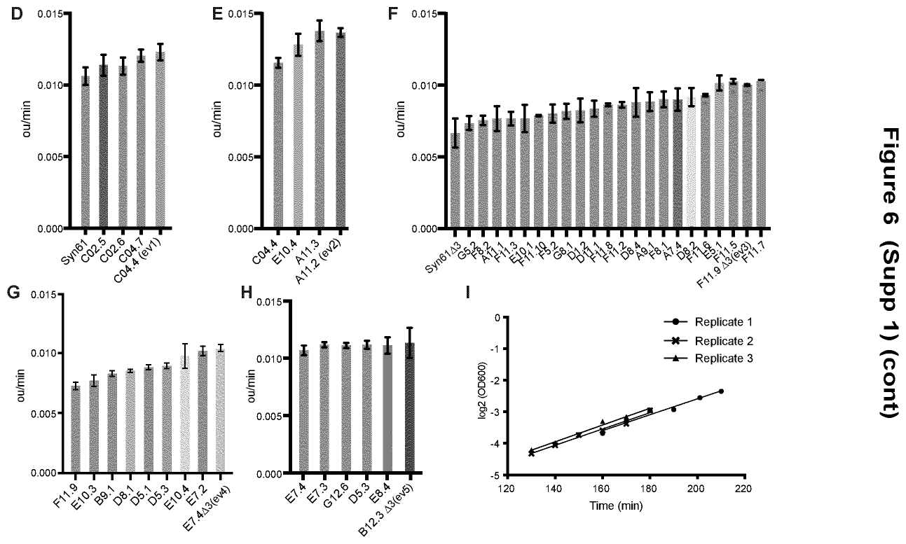

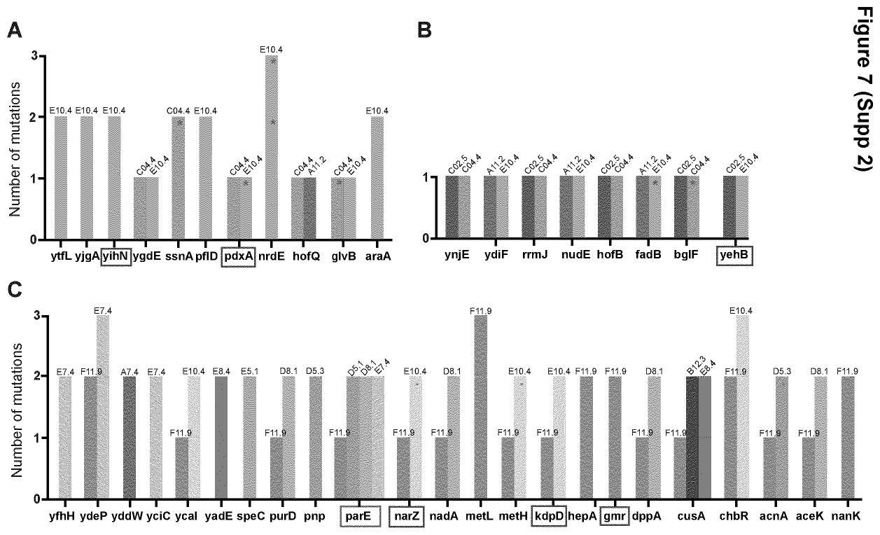

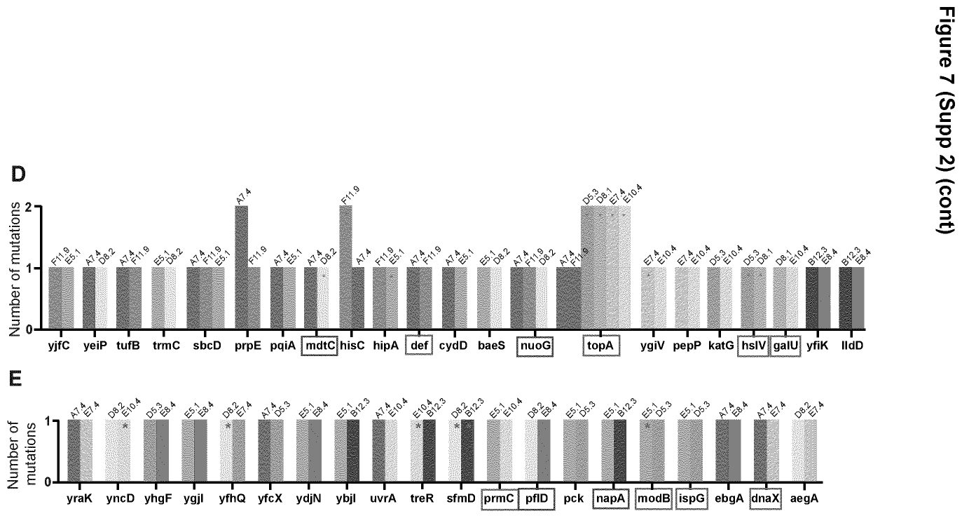

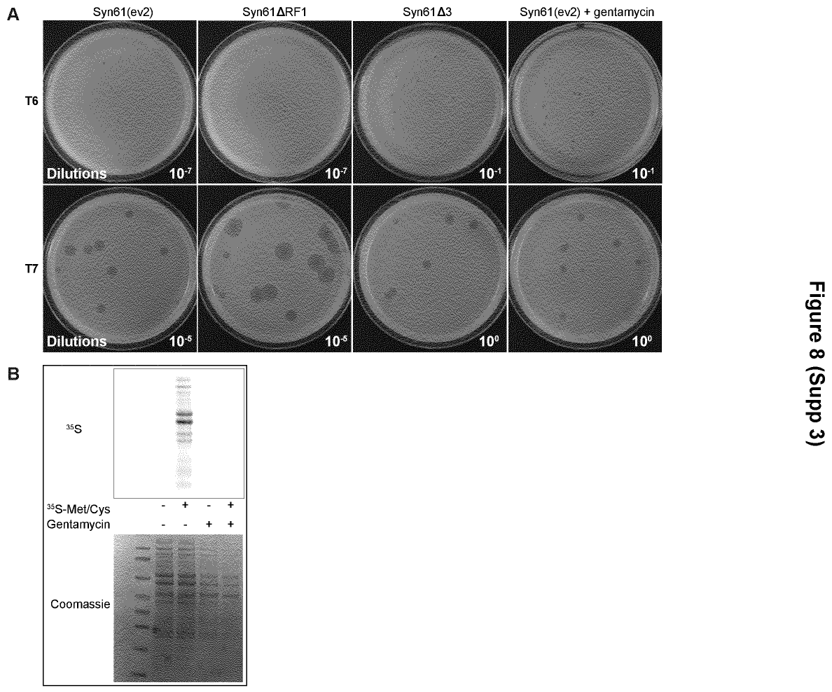

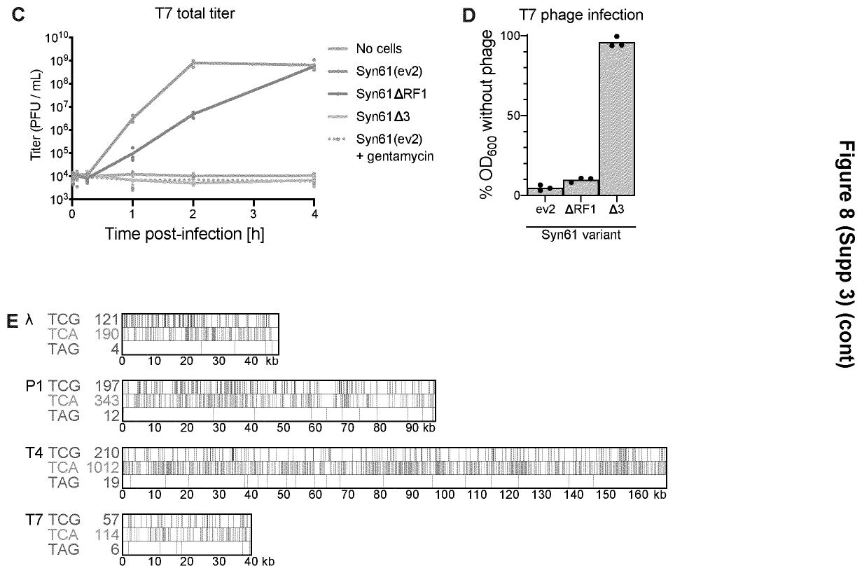

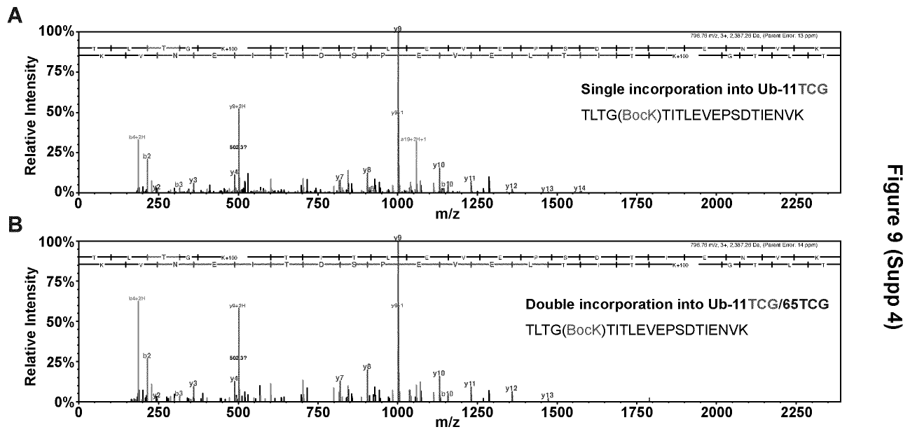

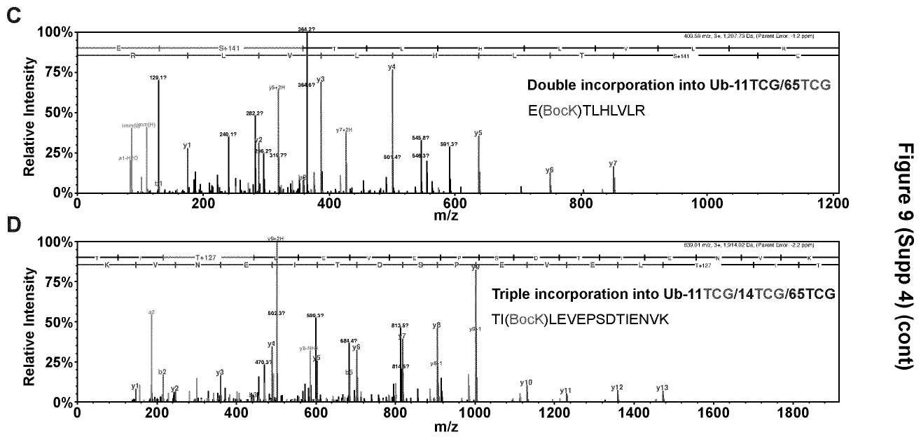

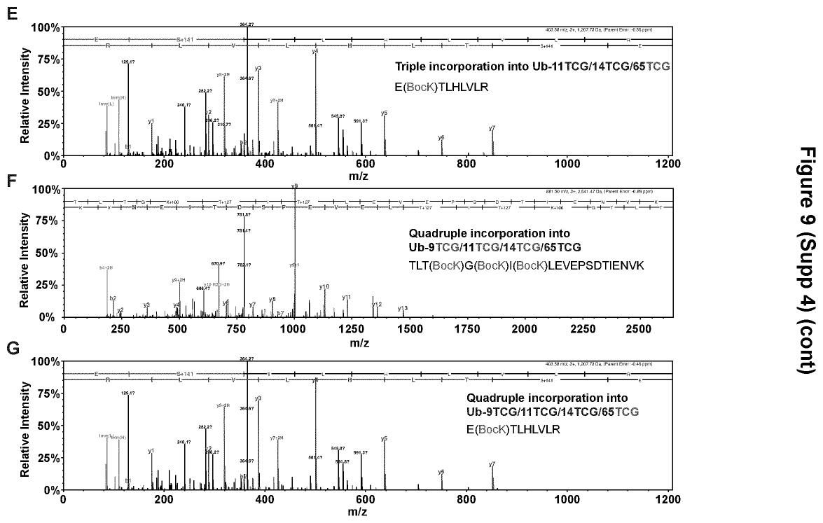

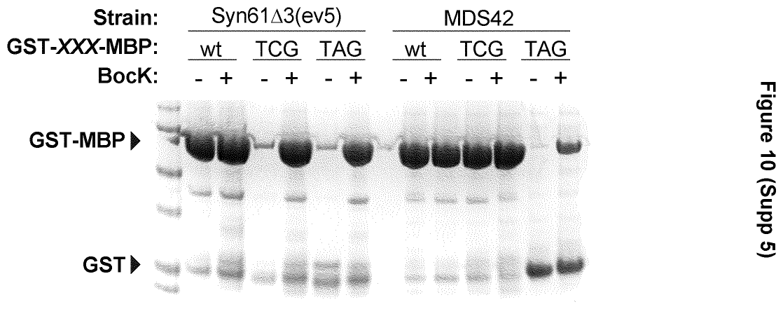

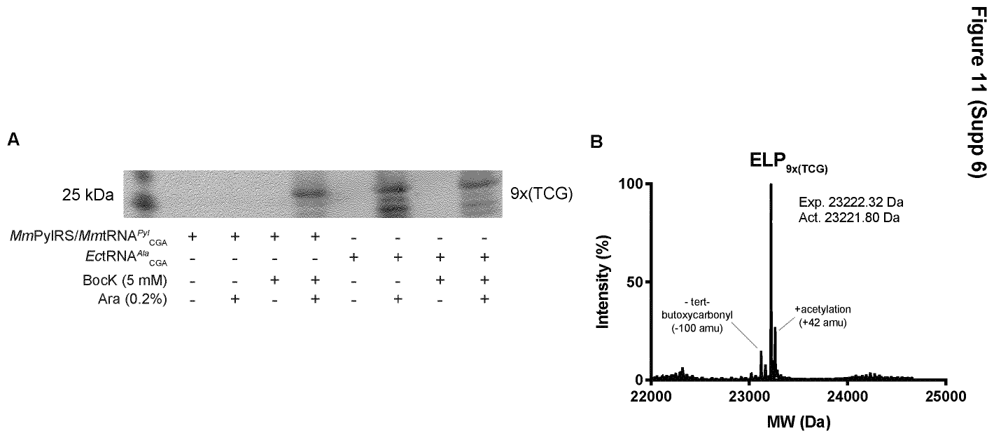

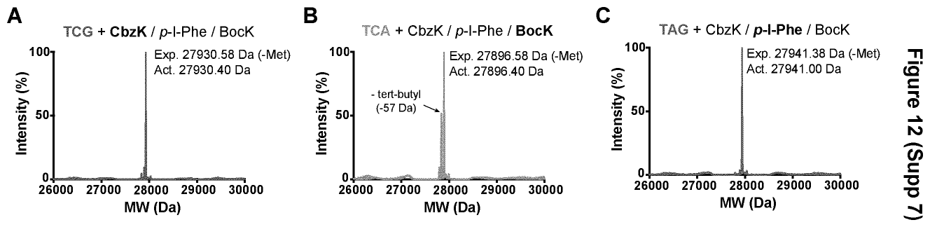

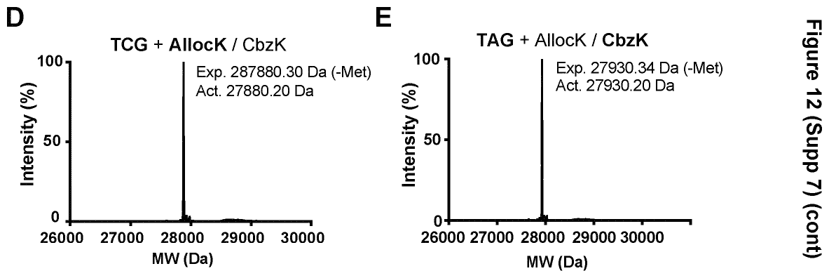

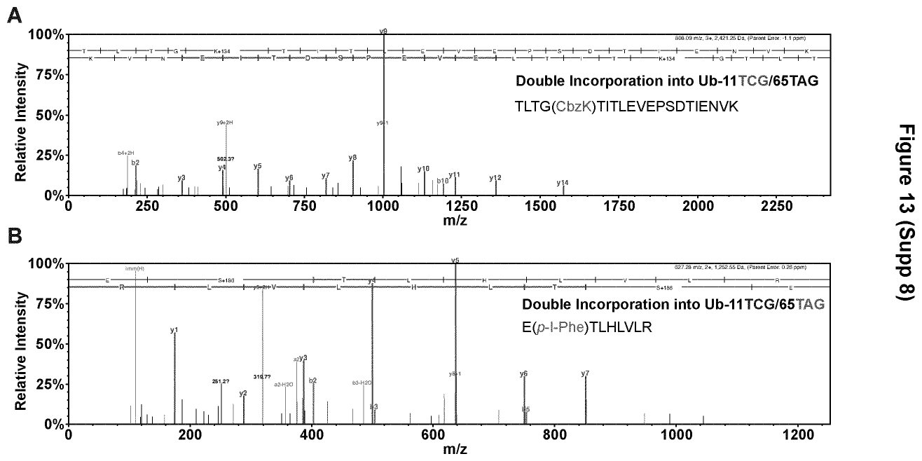

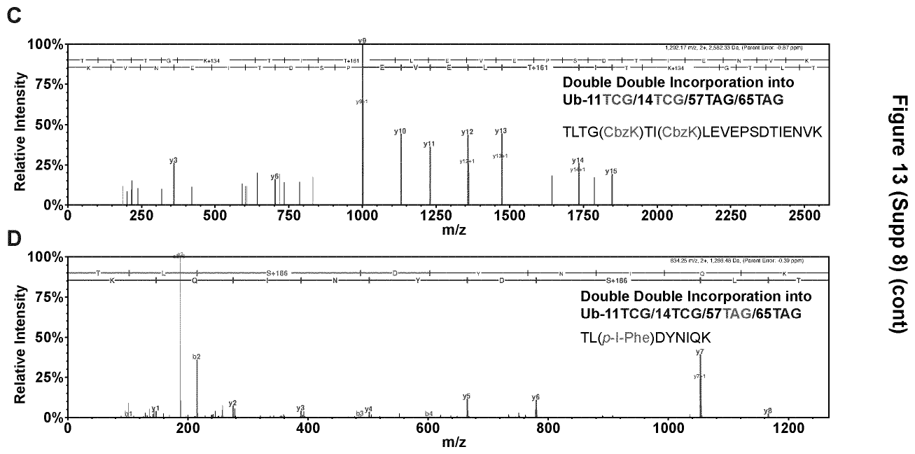

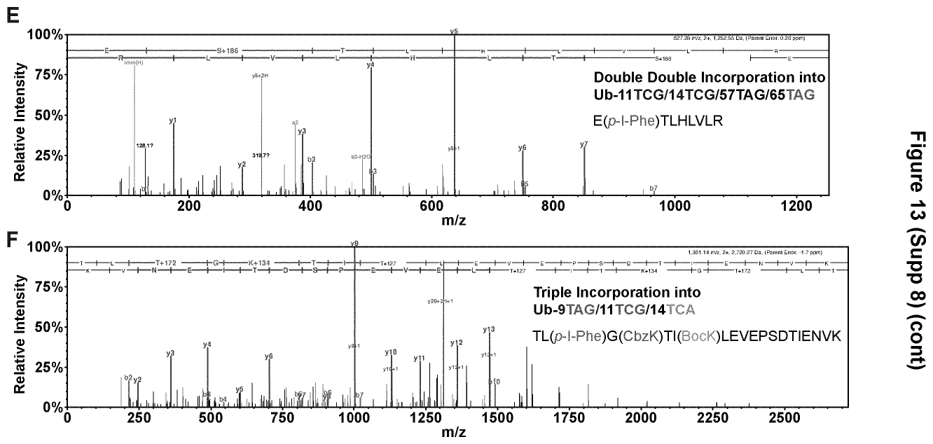

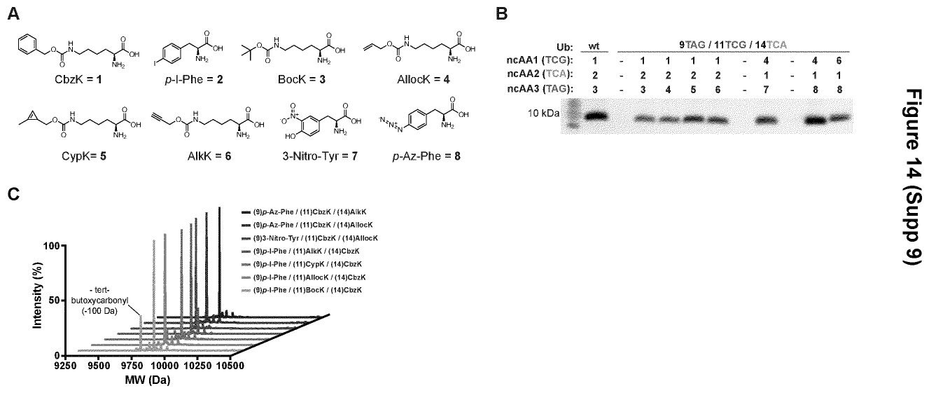

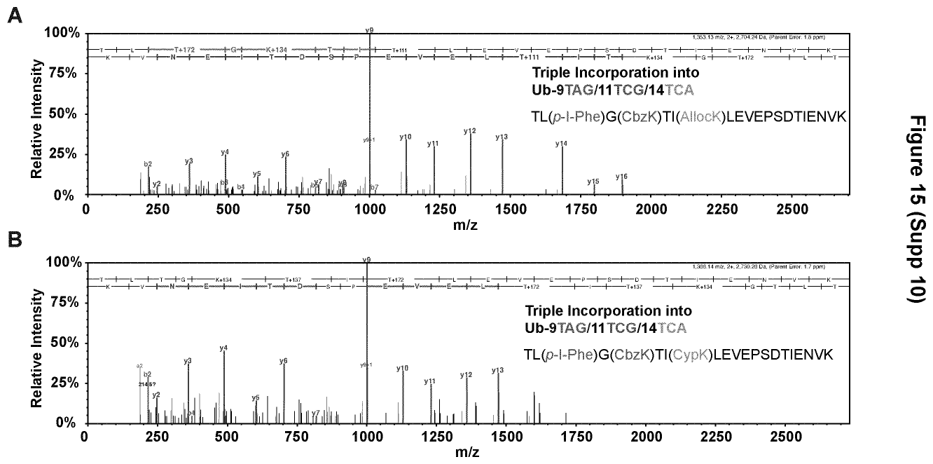

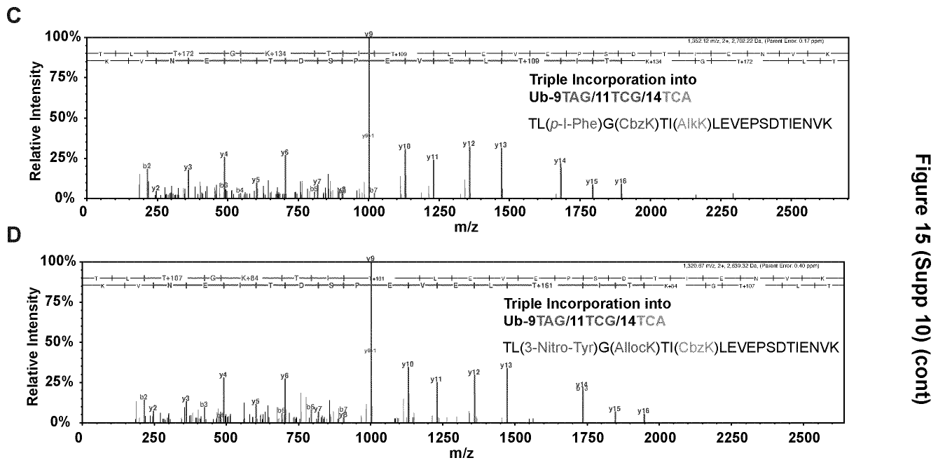

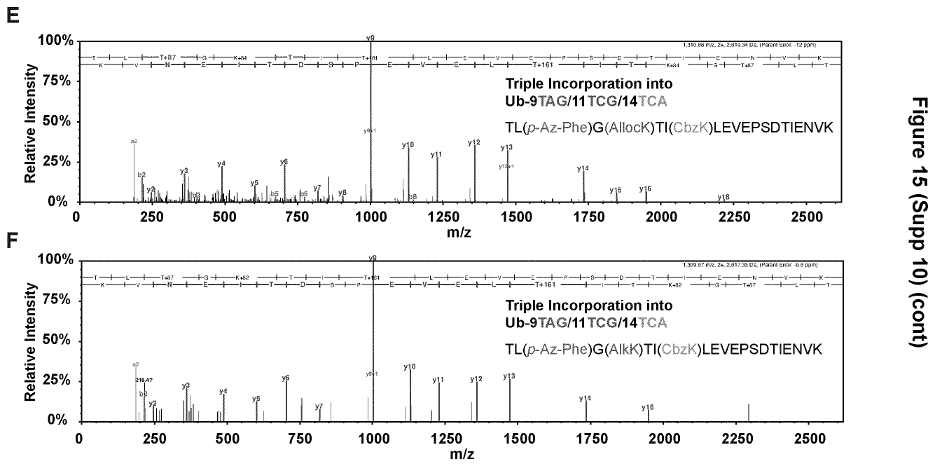

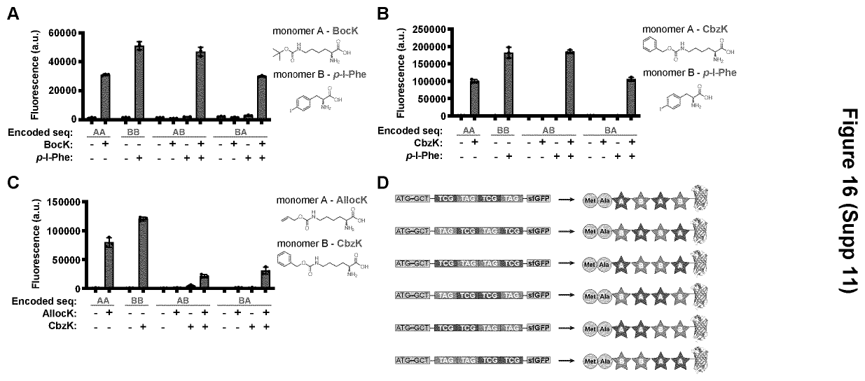

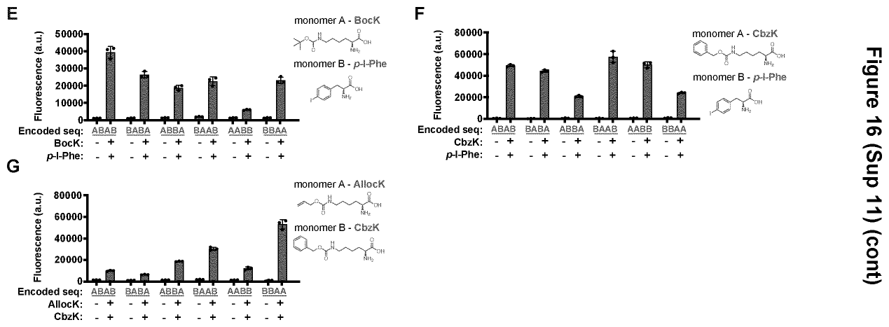

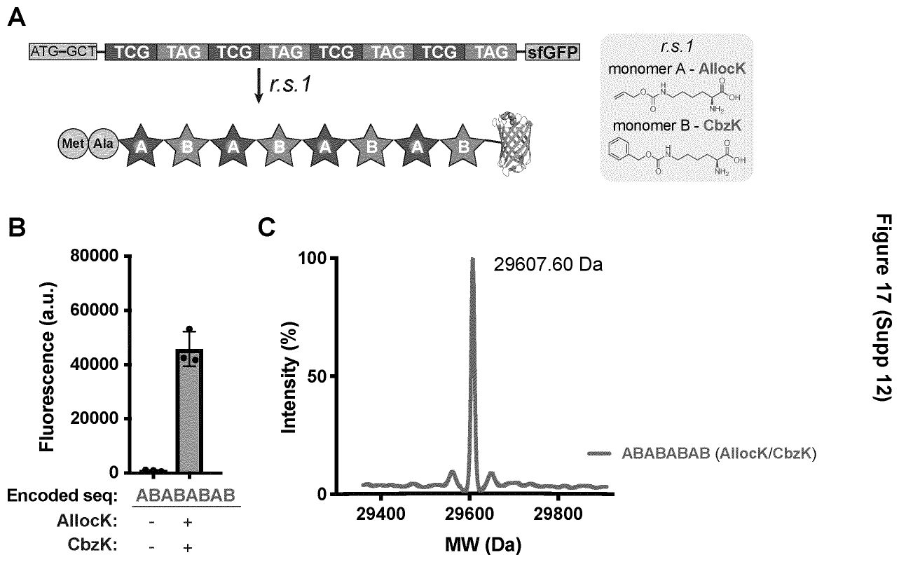

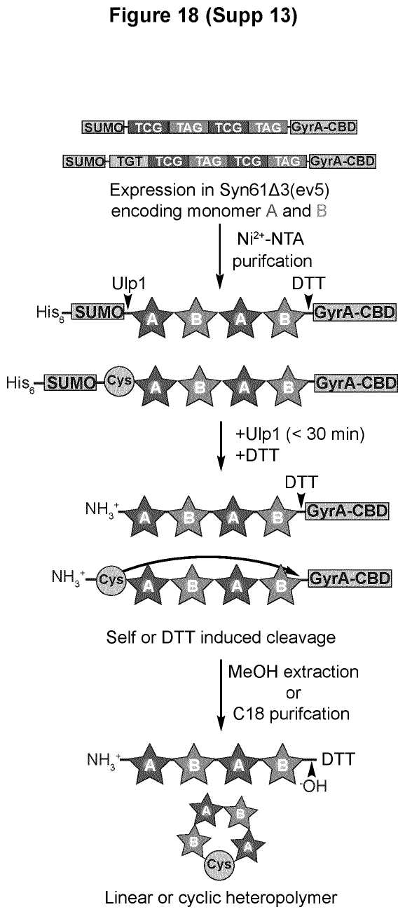

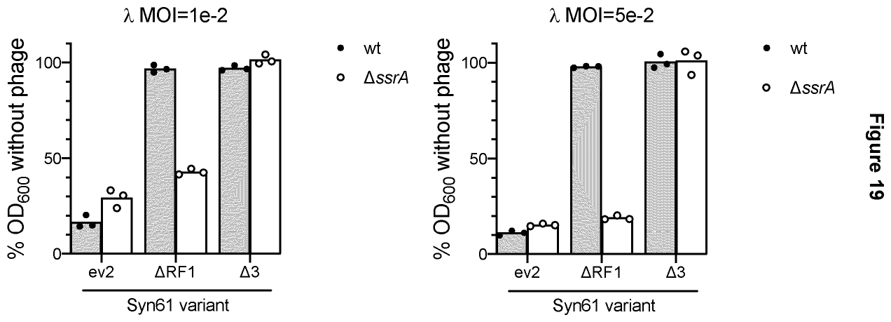

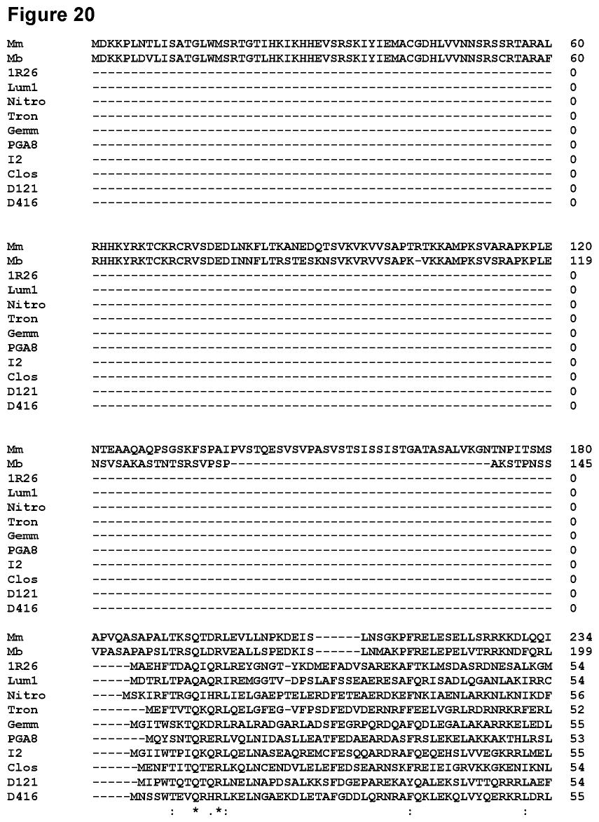

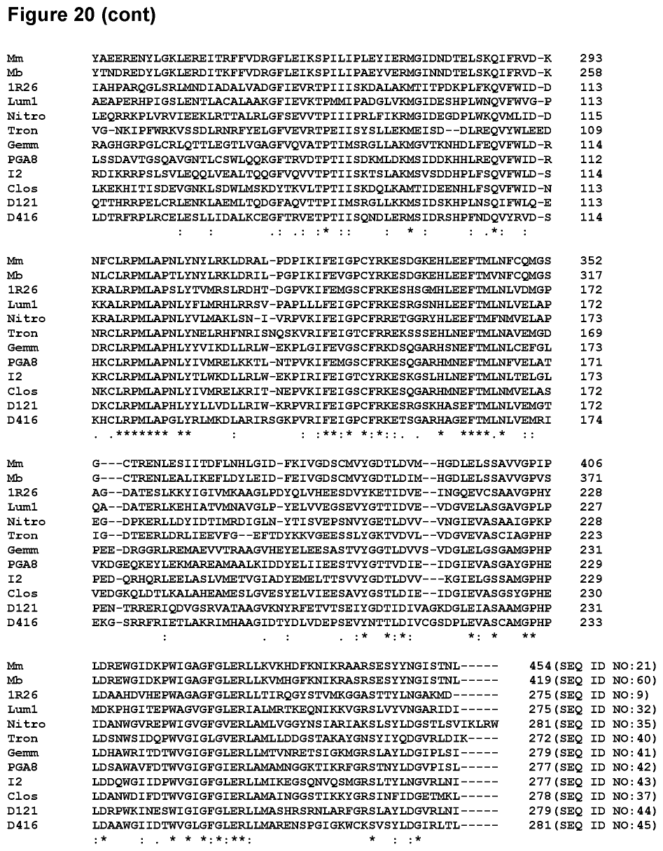

(A) Schematic of each codon reassignment. Introduction of an orthogonal aminoacyl-tRNA synthetase/tRNA_{YYY} pair—where YYY is the sequence of the anticodon of the orthogonal tRNA (encoded by O-tRNA)—to Syn61Δ3 (light teal box, as described in FIG. 1A) enables decoding of the cognate codon (XXX) introduced into a gene of interest. The orthogonal pair directs the incorporation of a non-canonical amino acid (ncAA) in response to the XXX codon. These codon reassignments are indicated in the dark grey box.

(B) TCG, TCA and TAG codons are not read by the translational machinery in Syn61Δ3, and codon reassignment enables ncAA incorporation into Ub_{11XXX}. Plasmids Encoding the orthogonal MmPylRS/MmtRNA^{Pyl}_{YYY} pair and a C-terminally His₆-tagged ubiquitin, with a single TCG, TCA, or TAG codon at position 11 (Ub_{11XXX}), or no target codons (wt) were introduced into Syn61Δ3. ‘XXX’ denotes a target codon and ‘YYY’ denotes a cognate anticodon. Expression of ubiquitin-His₆ was performed in the absence (−) or presence (+) of a ncAA substrate for MmPylRS, BocK. Full-length ubiquitin-His₆ was detected in cell lysate from an equal number of cells with an anti-His₆ antibody.

(C) Production of ubiquitin-His₆ incorporating BocK, Ub-(11BocK)-His₆, from a Ub_{11XXX} gene bearing the indicated target codon was confirmed by Electrospray ionization mass spectrometry (ESI-MS). Theoretical mass: 9487.7 Da; measured mass: 9487.8 Da (TCG), 9487.8 Da (TCA), 9488.0 (TAG). The peak of 100 Da smaller results from the loss of tert-butoxycarbonyl from BocK.

(D) As in panel (B) but using Ub_{11XXX,65XXX}, which contains target codons at positions 11 and 65 of the Ub gene.

(E) Production of ubiquitin-His₆ incorporating BocK at positions 11 and 65, from a Ub_{11XXX65XXX} gene bearing the indicated target codons was confirmed by ESI-MS. Theoretical mass: 9629.0 Da; measured mass: 9629.2 Da (TCG), 9629.0 Da (TCA), 9629.0 Da (TAG). The smaller peak of −100 Da corresponds to loss of tert-butoxycarbonyl from BocK.

(F) As in panel (B) but using Ub_{11XXX,14XXX,65XXX}, which contains target codons at positions 11, 14 and 65 of the Ub gene.

(G) Production of ubiquitin-His₆ incorporating BocK at positions 11, 14 and 65, from Ub_{11XXX,14XXX,65XXX} bearing the indicated target codons was confirmed by ESI-MS. Theoretical mass: 9756.1 Da; measured mass: 9756.2 Da (TCG), 9756.0 Da (TCA), 9756.0 Da (TAG). The smaller peaks of −100 Da or −200 Da correspond to loss of tert-butoxycarbonyl from one or two BocK residues, respectively.

(H) As in panel (B) but using Ub_{9XXX,11XXX,14XXX,65XXX}, which contains target codons at positions 9, 11, 14 and 65 of the Ub gene.

(I) Production of ubiquitin-His₆ incorporating BocK at positions 9, 11, 14 and 65, from Ub_{9XXX,11XXX,14XXX,65XXX} bearing the indicated target codons was confirmed by ESI-MS. Theoretical mass: 9883.3 Da; measured mass: 9883.2 Da (TCG), 9883.2 Da (TCA), 9883.2 Da (TAG). The smaller peaks of −100 Da or −200 Da correspond to loss of tert-butoxycarbonyl from one or two BocK residues, respectively. All experiments were performed in biological replicates three times with similar results.

FIG. 4. Double and triple incorporation of distinct non-canonical amino acids into TCG, TCA, and TAG codons in Syn61Δ3 cells.

(A) Reassignment of TCG (blue box), TCA (gold box) and TAG (green box) codons to distinct ncAAs in Syn61Δ3. Reassigning all three codons to distinct ncAAs in a single cell requires three engineered triply orthogonal aminoacyl-tRNA synthetase/tRNA pairs. Each pair must recognize a distinct ncAA and decode a distinct codon. The tRNAs from these triply orthogonal pairs are labelled O-tRNA¹⁻³.

(B) The incorporation of two distinct non-canonical amino acids in response to TCG and TAG codons in a single gene. Syn61Δ3(ev4)—containing the 1R26PylRS(CbzK)/AlvtRNA^{ΔNPyl(8)}_{CGA} pair and the AfTyrRS(p-I-Phe)/AftRNA^{Tyr(A01)}_{CUA} pair (30) (which direct the incorporation of N^{ε}-(carbobenzyloxy)-L-lysine (CbzK) into TCG and (S)-2-Amino-3-(4-iodophenyl)propanoic acid (p-I-Phe) into TAG, respectively)—were provided with CbzK and p-I-Phe. Cells also contained Ub_{11TCG,65TAG} (TCG/TAG) or Ub9_{TCG,11TCG,14TAG,65TAG} (2×TCG/2×TAG), or wt Ub, which contains no target codons. Expression of ubiquitin-His₆ was performed in the absence (−) or presence (+) of the ncAAs. Full-length ubiquitin-His₆ was detected in cell lysate from an equal number of cells with an anti-His₆ antibody.

(C) ESI-MS analyses of purified Ub-(11CbzK, 65p-I-Phe) (black trace) and Ub-(11CbzK, 14CbzK, 57p-I-Phe, 65p-I-Phe) (grey trace), expressed in the presence of CbzK and p-I-Phe, as described in panel (E) and purified by Ni²⁺-NTA chromatography. These data confirm the quantitative incorporation of CbzK and p-I-Phe in response to TCG and TAG codons, respectively. Ub-(11CbzK, 65p-I-Phe), theoretical mass: 9707.81 Da; measured mass: 9707.40 Da. Ub-(11CbzK, 14CbzK, 57p-I-Phe, 65p-I-Phe), theoretical mass: 10055.00 Da; measured mass: 10055.60 Da.

(D) The incorporation of three distinct non-canonical amino acids into TCG, TCA and TAG codons in a single gene. Syn61Δ3(ev4)—containing the 1R26PylRS(CbzK)/AlvtRNA^{ΔNPyl(8)}_{CGA} pair, the MmPylRS/MmtRNA^{Pyl}_{UGA} pair and the AfTyrRS(p-I-Phe)/AftRNA^{Tyr(A01)}_{CUA} pair—were provided with CbzK, BocK and p-I-Phe. Cells also contained Ub_{9TAG,11TCG,14TCA} (TCG/TCA/TAG). Expression of this gene was performed in the absence (−) or presence (+) of the ncAAs. Full-length Ub-(9p-I-Phe, 11 CbzK, 14BocK)-His₆ was detected in cell lysate from an equal number of cells with an anti-His₆ antibody. (E) ESI-MS of purified Ub-(9p-I-Phe, 11CbzK, 14BocK), theoretical mass: 9820.97 Da; measured mass: 9820.80 Da. Western blot experiments (panels (B) and (D)) were performed in 5 biological replicates with similar results. The ESI-MS data (panels (C) and (E)) was collected once.

FIG. 5. Programmable, encoded synthesis of non-canonical heteropolymers and macrocycles

(A) Elementary steps in the ribosomal polymerization of two distinct ncAA monomers (labelled A (dark blue) and B (green)). All linear heteropolymer sequences composed of A and B can be encoded from these four elementary steps.

(B) Encoding heteropolymer sequences (non-canonical monomers are shown as stars). The sequence of monomers in the heteropolymer is programmed by the sequence of codons written by the user. The identity of monomers (A and B) is defined by the aminoacyl-tRNA synthetase/tRNA pairs added to the cell. Cells can be reprogrammed to encode different heteropolymer sequences from a single DNA sequence. Sequences were encoded as insertions at position 3 of sfGFP-His6. Reassignment scheme 1 (r.s.1) uses the MmPylRS/MmtRNA^{Pyl}_{CGA} pair to assign AllocK as monomer A and the 1R26PylRS(CbzK)/AlvtRNA^{ΔNPyl(8)}_{CUA} pair to assign CbzK as monomer B (Fig. S7, D to E). r.s.2 uses the MmPylRS/MmtRNA^{Pyl}_{CGA} pair to assign BocK as monomer A and an AfTyrRS(p-I-Phe)/AftRNA^{Tyr(A01)}_{CUA} pair to assign p-I-Phe as monomer B. r.s.3 uses the 1R26PylRS(CbzK)/AlvtRNA^{ΔNPyl(8)}_{CGA} pair to assign CbzK as monomer A and the AfTyrRS(p-I-Phe)/AftRNA^{Tyr(A01)}_{CUA} pair to assign p-I-Phe as monomer B.

(C-E) Polymerization of the encoded sequence composed of the indicated ncAAs, and the resulting sfGFP-His6 expression in Syn61Δ3(ev5) were dependent on the addition of both ncAAs to the medium.

(F) Electrospray ionization (ESI) MS of purified sfGFP-His6 variants containing the indicated ncAA hexamers. BocK/p-I-Phe (expected mass after loss of N-terminal methionine: 29172.07 Da; observed: 29171.8 Da), CbzK/p-I-Phe (expected mass after loss of N-terminal methionine: 29274.13 Da; observed: 29274.0 Da) and AllocK/CbzK (expected mass after loss of N-terminal methionine: 29091.64 Da; observed: 29092.2 Da). The ESI-MS data was collected once.

(G) Encoded synthesis of free non-canonical polymers. DNA sequences encoding a tetramer and a hexamer were inserted between SUMO and a GyrA intein coupled to a chitin-binding domain (CBD), in Syn61Δ3(ev5) cells containing the same pairs as in r.s.1 (B). Expression of the constructs, followed by Ulp1 cleavage and GyrA trans-thioesterification cleavage, results in the isolation of free non-canonical tetramer and hexamer polymers. Adding an additional cysteine immediately upstream of the polymer sequence results in self-cleavage and release of a macrocyclic non-canonical polymer.

(H-J) Chemical structures and ESI-MS spectra of the purified linear and cyclic AllocK/CbzK heteropolymers. The raw ESI-MS spectra show the relative intensity and observed m/z ratios for the different non-canonical peptides. The observed masses corresponding to the expected [M+H]⁺ or [M+2H]²⁺ ions are highlighted in bold. Other adducts and fragment ions are labelled relative to these.

FIG. 6 (S1). Syn61 strain evolution and knockouts.

(A) Schematic of automated parallel evolution. Clonal cultures are shown in pale yellow; mutagenized populations in each well are represented by distinct shades. Mutator cultures were grown for 17-70 generations with induced random mutagenesis followed by six cycles of dynamic parallel selection on a robotics platform. Regular assessment of optical density (OD₆₀₀) and re-dilution of all wells when 3 wells reach an OD₆₀₀ of 0.5 enables continuous growth in exponential phase for approximately 52 generations. Finally, single colonies are isolated from mutagenized populations and characterized by growth measurement and whole genome sequencing. Figure adapted from Schmied et al. (17) with permission. (B) Lineage of Syn61 and Syn61Δ3 descendants isolated from evolved pools. Colored blocks represent individual clones isolated from independently evolved pools after each round, underlined clones were selected for subsequent rounds of evolution. Grey blocks represent clones that were characterized by growth, with some sequenced (C02.6, C04.7, A11.3, E7.2) but not considered for further ORF mutation analysis in Fig. S2. A list with all mutations identified in sequencing analyses of the evolved strains is provided in Data file S1. The 16 additional clones not indicated for round 1 with Syn61Δ3 are G5.2, F8.2, A11.1, F11.3, E10.1, F11.10, F5.2, G8.1, D1.2, D11.1, F11.8, F11.2, D8.4, A9.1, F8.1, F11.6.

(C) Schematic of strain development from Syn61 to Syn61Δ3(ev5). The codons that encode serine and protein termination are connected to the anticodons of the tRNAs or release factors predicted to decode them by black lines; all sequences are written 5′ to 3′. The genes encoding the corresponding tRNAs and release factors are indicated in the black boxes. The inventors performed automated parallel mutagenesis and dynamic selection to create a faster growing version of Syn61, Syn61(ev1). This strain was then further evolved to Syn61(ev2). Mutations gained in each strain are separated as mutations in open reading frames (ORFs), non-coding mutations and mutations in intergenic regions. For a detailed list of all mutations gained in the strain development see Data file S1. Targeted deletion of serT, serU, and prfA created Syn61Δ3. In this process the inventors observed rearrangements at 2 loci: i) the collapse of a CRISPR array leading to a 429 bp deletion and ii) the partial replacement of ribosomal operon rrnC with a duplicated segment from the highly homologous ribosomal operon rrnB. The corresponding changes relative to wt Syn61 amount to 19 mutations. After three more subsequent rounds of evolution, the inventors isolated Syn61Δ3(ev3), Syn61Δ3(ev4), and Syn61Δ3(ev5), respectively.

(D) Growth rates of four clones isolated with increased growth rate after one round of Syn61 evolution with mutagenesis for ˜17 generations. Two out of 96 parallel evolved pools (C02 and C04) yielded clones with improved growth. Sequences of clones derived from the same pool (C02.5 and C02.7 or C04.7 and C04.4) were nearly identical, thus they were considered clonal. Open reading frames with more than one mutation are listed in Fig. S2. Growth rates were calculated for n=8 replicate cultures for each strain. For statistics, see Methods.

(E) Growth rates of three clones isolated with increased growth rate after the second round of Syn61 evolution, starting with mutagenesis of C04.4 (Syn61(ev1)) for ˜17 generations. Two out of 96 parallel evolved pools (E10 and A11) yielded clones with improved growth. Growth rates were calculated for n=8 replicate cultures for each strain. For statistics, see Methods.

(F) Growth rates of 22 clones isolated with increased growth rate after one round of Syn61Δ3 evolution with mutagenesis for ˜70 generations, derived from 11 independent pools. Growth rates were calculated for n=5 replicates or n=1 (denoted with red asterisk) cultures for each strain. For statistics, see Methods.

(G) Growth rates of 7 clones isolated from independent pools with increased growth rate after the second round of Syn61Δ3 evolution, starting with mutagenesis of F11.9 (Syn61Δ3(ev3)) for ˜45 generations. Growth rates were calculated for n=6 replicate cultures for each strain. For statistics, see Methods.

(H) Growth rates of 5 clones isolated from independent pools with increased growth rate after the third round of Syn61Δ3 evolution, starting with mutagenesis of E7.4 (Syn61Δ3(ev4)) for ˜60 generations. Growth rates were calculated for n=6 replicate cultures for each strain. For statistics, see Methods.

(I) The doubling time of the most evolved Syn61 derivative (Syn61Δ3(ev5)) was calculated from growth curves recorded under standard laboratory conditions. Three independent cultures were grown in shake flasks at 37° C. and OD₆₀₀ measurements were taken every 10 minutes during exponential growth. For each independent culture a 60 min window during exponential phase was chosen to calculate an average growth rate of 0.026±5·10⁻⁴ and a doubling time of 38.72±1.02 min for Syn61Δ3(ev5). For statistics, see Methods.

FIG. 7 (S2). Open reading frames mutated more than once during Syn61 evolution.

(A) For this analysis the inventors selected 14 clones, one sequenced clone from each pool, denoted with colored blocks in Fig S1B. The inventors identified open reading frames mutated more than once, between Syn61 and Syn61Δ3, in a single strain or evolutionarily related strains. Mutations arising in a single strain or in strains related through evolutionary history may not be independent. For example, a detrimental frameshift mutation introduced in nrdE in E10.4 may mean that additional mutations in nrdE are not selected for or against, as the gene is already inactivated. The inventors observed that on average 2*10⁻⁶ mutations per bp per generation were introduced with induced random mutagenesis. On average we accumulated 87.25 mutations in ORFs within the genome per round of mutagenesis and selection. If each mutation is assumed to be independent, then the probability of finding at least two mutation in the same open reading frame (given 3556 ORFs in E. coli) is 0.65. Therefore, it is not unlikely that these mutations are found in the same ORF by chance. Our data are consistent with their being a wide variety of ways for a recoded strain to improve its fitness. Red asterisks denote detrimental mutations, including premature stop codons and frameshifts due to insertion or deletion of typically 1 bp. Red boxes mark essential genes, green boxes in panel (A) and (B) mark genes affected with one additional mutation during post-knockout evolution. A list with all mutations identified in sequencing analyses of the evolved strains is provided in Data file S1.

(B) Open reading frames mutated more than once, between Syn61 and Syn61Δ3, in independently derived strains. rrmJ, a 23S rRNA methyltransferase, gained two nonsense mutations during initial evolution of Syn61 within 40 bp of each other (P190L, V203A), in close proximity to a Syn61 recoding event (S197). On average we accumulated 87.25 mutations in ORFs within the genome per round of mutagenesis and selection. If each mutation is assumed to be independent, then the probability of finding at least two mutation in the same open reading frame (given 3556 ORFs in Syn61) is 0.65. Therefore, it is not unlikely that these mutations are found in the same ORF by chance. Overall, there was minimal overlap between the mutations arising in independently evolved strains. Our data are consistent with their being a wide variety of ways for a recoded strain to improve its fitness.

(C) Open reading frames mutated more than once between Syn61Δ3 and Syn61Δ3(ev5) in a single strain or related strains. parE, an essential gene involved in chromosome segregation, accumulated the overall highest number of mutations. Green boxes in panel (C), (D) and (E) mark genes affected with one additional mutation in a strain derived during Syn61 evolution before knockout of the decoding elements. On average we accumulated 62.3 mutations in ORFs within the genome per round of mutagenesis and selection. If it is assumed that each mutation is independent, then the probability of finding at least two mutation in the same open reading frame (given 3556 ORFs in Syn61) is 0.41. Therefore, it is not unlikely that these mutations are found in the same ORF by chance. Overall, there was minimal overlap between the mutations arising in independently evolved strains. These data are consistent with their being a wide variety of ways for a recoded strain to improve its fitness.

(D) Open reading frames mutated more than once between Syn61Δ3 and Syn61Δ3(ev5) in independently derived strains within the same evolution round. ORFs affected within the same evolution round in independently evolved strains may indicate a strong evolutionary pressure on the predecessor. Four identical mutations were recorded in topA in all strains derived after round 2. The inventors suspect that the 1 bp deletion occurred after sequencing F11.9 before splitting the strain into separate mutagenesis pools. sbcD and nuoG are the most affected by independent mutations in the initial evolution of Syn61Δ3. Overall, there was minimal overlap between the mutations arising in independently evolved strains, indicating a wide variety of ways for a recoded strain to improve its fitness. On average we accumulated 62.3 mutations in ORFs within the genome per round of mutagenesis and selection. If it is assumed that each mutation is independent, then the probability of finding at least two mutation in the same open reading frame (given 3556 ORFs in Syn61) is 0.41. Therefore, it is not unlikely that these mutations are found in the same ORF by chance. Overall, there was minimal overlap between the mutations arising in independently evolved strains. These data are consistent with their being a wide variety of ways for a recoded strain to improve its fitness.

(E) Open reading frames mutated more than once between Syn61Δ3 and Syn61Δ3(ev5) in independently derived strains in separate evolution rounds. No more than two mutations emerged independently across rounds of Syn61Δ3 evolution. On average we accumulated 62.3 mutations in ORFs within the genome per round of mutagenesis and selection. If it is assumed that each mutation is independent, then the probability of finding at least two mutation in the same open reading frame (given 3556 ORFs in Syn61) is 0.41. Therefore, it is not unlikely that these mutations are found in the same ORF by chance. Overall, there was minimal overlap between the mutations arising in independently evolved strains. These data are consistent with their being a wide variety of ways for a recoded strain to improve its fitness.

FIG. 8 (S3). Phage propagation is ablated in Syn61Δ3.

(A) Relative total phage titer after 4 hours of infection of Syn61 variants (see FIG. 2C). The titer of free and intracellular phage resulting from infection was determined by isolating phage from the cultures, serially diluting the isolate, and using it to infect E. coli MG1655 on plates. Cultures of Syn61Δ3 and gentamycin-treated Syn61(ev2), in which protein synthesis, and therefore phage production, is inhibited, contain the same amount of phage after 4 hours, while Syn61(ev2) and Syn61ΔRF-1 propagate the phage and lead to infection. The shown plates are the indicated dilutions.

(B) Protein synthesis is ablated in gentamycin-treated cells. A Syn61(ev2) control and Syn61(ev2) treated with gentamycin were grown with ³⁵S-Met/Cys and labeled proteins were visualised by SDS-PAGE and phosphorimaging. Despite exposing the phosphor screen for two weeks, no signal was observed for the gentamycin-treated sample.

(C) Total titer of T7 phage upon infecting Syn61 and its derivatives. Cultures were infected with phage at MOI=5·10⁻⁵ (multiplicity of infection) and the total titer was monitored over 4 hours.

(D) T7 efficiently lyses Syn61 variants but not Syn61Δ3. Cultures were infected as in panel (C) and OD₆₀₀ was measured after 4 hours.

(E) Schematic of the TCG, TCA, and TAG codon density in the genomes of phage λ, P1, T4, and T7, as in FIG. 2B.

FIG. 9 (S4). MS-MS spectra confirm site-specific non-canonical amino acid (ncAA) incorporation into reporters containing one, two, three or four TCG codons in Syn61Δ3.

(A) Ub_{11TCG} reporter containing BocK at position 11 (Ub-(11BocK)) (see FIGS. 3, B and C) was purified by Ni²⁺-NTA and subject to trypsin digestion followed by LC/MS-MS analysis (see Methods for full details). The tryptic peptides confirm the site-specific incorporation of BocK into the TCG codon at position 11.

(B-C) LC/MS-MS analysis of Ub-(11BocK, 65BocK) (see FIGS. 3, D and E), as in (A). The tryptic peptides confirm the site-specific incorporation of BocK into TCG codons at positions 11 (B) and 65 (C).

(D-E) LC/MS-MS analysis of Ub-(11BocK, 14BocK, 65BocK) (see FIGS. 3, F and G), as in (A). The tryptic peptides confirm the site-specific incorporation of BocK into TCG codons at positions 11, 14 (D) and 65 (E).

(F-G) LC/MS-MS analysis of Ub-(9BocK, 11BocK, 14BocK, 65BocK) (see FIGS. 3, H and I), as in (A). The tryptic peptide containing residues 9, 11, and 14 (F) was found with the correct parent ion mass which harboured three BocK masses. The fragmentation pattern of this parent ion confirmed the site-specific incorporation of BocK into TCG codons at positions 11 and 14, though there were not enough b- and y-series ions to localize BocK to position 9. (G) The tryptic peptide confirming the site-specific incorporation of BocK into the TCG codon at position 65.

FIG. 10 (S5). GST-MBP comparison of sense codon and amber codon suppression in Syn61Δ3(ev5).

Syn61Δ3(ev5) cells harbouring an orthogonal aaRS/tRNA pair and its corresponding ncAA efficiently generate full-length GST-MBP protein in response to a sense codon or amber codon. The inventors co-transformed Syn61Δ3(ev5) cells or MDS42 cells (control) with a plasmid encoding the orthogonal MmPylRS/MmtRNA^{Pyl}_{YYY} pair and a plasmid encoding the cognate GST-XXX-MBP reporter; XXX encodes either a wildtype (wt) codon, a TCG sense codon, or a TAG stop codon, and YYY denotes its cognate anticodon. Following induction for GST-XXX-MBP expression in the absence (−) or presence (+) of BocK (5 mM), expressed protein was pulled down with glutathione sepharose beads. Bound samples were then eluted with reduced glutathione and the resultant full-length GST-MBP or truncated GST products analyzed by SDS-PAGE. For both the TCG sense codon and TAG stop codon in Syn61Δ3(ev5) cells, efficient GST-MBP full-length synthesis was detected only in the presence of BocK (see Data file S4 for yields).

FIG. 11 (S6). Syn61Δ3(ev4) enables reassignment of nine sense codons to a non-canonical amino acid (ncAA) in an elastin-like polypeptide (ELP).

(A) Syn61Δ3(ev4) enables multi-site ncAA incorporation into the 9 TCG codons in ELP_{9x(TCG)}. The inventors co-transformed a plasmid encoding the orthogonal MmPylRS/MmtRNA^{Pyl}_{CGA} pair and a plasmid encoding ELP₉ₓ(T_{CG}); this contains nine repeats of the ELP coding sequence, with each repeat containing a TCG codon, and a His₆-tag is encoded at the 3′ end. Each ELP repeat of ‘VPGVGVPGVGVPGXGVPGVGVPGVG’ (SEQ ID NO: 1). The sequence of the plasmid is GenBank accession MW879732, and provided herein as SEQ ID NO: 2) contains 24 canonical amino acids and one ncAA residue (X). In cells transformed with the MmPylRS/MmtRNA^{Pyl}_{CGA} plasmid, ELP_{9x(TCG)} expression was induced in the absence (−) or presence (+) of BocK. The inventors then used an anti-His₆ antibody to monitor for full-length protein (ELP_{9x(BocK)}) production from normalized cell lysates—efficient ELP_{9x(BocK)} synthesis was detected only in the presence of both arabinose (Ara) and BocK. As a comparison for the efficiency of the orthogonal aaRS/tRNA pair, cells were separately transformed with a plasmid encoding EctRNA^{Ala}_{CGA}, a chimeric alanyl tRNA with a CGA anticodon which directs alanine incorporation in response to TCG codons, from which the inventors detected efficient ELP_{9x(Ala)} synthesis in the presence of arabinose (Ara). Western blots were performed in biological replicates three times with similar results.

(B) ESI-MS of Ni²⁺-NTA purified ELP_{9x(BocK)}-His₆ from the experiment described in (A) confirms the incorporation of BocK into 9 TCG codons. Theoretical mass: 23222.32 Da; measured mass: 23221.80 Da. ESI-MS data was collected once.

FIG. 12 (S7). Mutual orthogonality of the aaRS/tRNA pairs used for double and triple noncanonical amino acid incorporations.

(A-C) The aaRs/tRNA pairs used for double and triple distinct non-canonical amino acid incorporations in FIG. 4 and FIG. 5 are mutually orthogonal. Syn61Δ3(ev4)—containing the 1R26PylRS(CbzK)/AlvtRNA^{ΔNPYl(8)}_{CGA} pair, the MmPylRS/MmtRNA^{Pyl}_{UGA} pair and the AfTyrRS(p-I-Phe)/AftRNA^{Tyr(A01)}_{CUA} pair (which direct the incorporation of CbzK into TCG, BocK into TCA and p-I-Phe into TAG, respectively)—was provided with CbzK, BocK and p-IPhe. Cells also contained GFP_{3TCG} (panel A), GFP_{3TCA} (panel B), or GFP_{3TAG} (panel C). Electrospray ionization (ESI) MS analysis of GFP-His₆ purified from cells containing GFP_{3TCG} incorporated CbzK, but not BocK or p-I-Phe. GFP_{3TCA} led to the incorporation of BocK, and GFP_{3TAG} led to incorporation of p-I-Phe.

(D, E) The MmPylRS/MmtRNA^{Pyl}_{CGA} and 1R26PylRS(CbzK)/AlvtRNA^{ΔNPYl(8)}_{CUA} pairs incorporate distinct ncAAs in response to distinct codons in Syn61Δ3(ev4). The orthogonal aaRS/tRNA pairs MmPylRS/MmtRNA^{Pyl}_{CGA} and 1R26PylRS(CbzK)/AlvtRNA^{ΔNPyl(8)}_{CUA} each incorporate their distinct, cognate ncAA in response to their distinct, cognate codons. In the presence of both MmPylRS/MmtRNA^{Pyl}_{CGA} and 1R26PylRS(CbzK)/AlvtRNA^{ΔNPyl(8)}_{CUA} in Syn61Δ3(ev4) cells harbouring a GFP_{3XXX} reporter plasmid, the inventors expressed GFP in the presence of both AllocK and CbzK (+/+). (D) ESI-MS of Ni²⁺-NTA purified GFP_{3TCG} yielded a mass corresponding to the incorporation of AllocK, and not CbzK, thus demonstrating the orthogonality of MmPylRS/MmtRNA^{Pyl}_{CGA} for its cognate ncAA and cognate codon. (E) ESIMS of Ni²⁺-NTA purified GFP_{3TAG} yielded a mass corresponding to the incorporation of CbzK, and not AllocK, thus demonstrating the orthogonality of the 1R26PylRS(CbzK)/AlvtRNA^{ΔNPyl(8)}_{CUA} pair for its cognate ncAA and cognate codon.

FIG. 13 (S8). MS-MS spectra confirm the site-specific incorporation of distinct non-canonical amino acids (ncAAs) into double, double-double, and triple ubiquitin reporters in Syn61Δ3(ev4).

(A, B) Ub-(11CbzK, 65p-I-Phe) double (see FIG. 4B), Ub-(11CbzK, 14CbzK, 57p-I-Phe, 65p-IPhe) double-double (see FIG. 4B), and Ub-(9p-I-Phe, 11CbzK, 14BocK) triple (see FIG. 4D) ncAA incorporations were purified by Ni²⁺-NTA and subjected to LC-MS/MS analyses following trypsin digestion to confirm the site-specific incorporation of distinct ncAAs into distinct sense and stop codons (see Methods for full details). For double distinct ncAA incorporation into the double Ub_{11TCG,65TAG} gene, the purified protein Ub-(11CbzK, 65p-I-Phe) yielded tryptic peptides corresponding to the site-specific incorporation of CbzK into the 11TCG codon (A) and p-I-Phe into the 65TAG codon (B).

(C, D, E) For double-double distinct ncAA incorporation into the Ub_{11TCG,14TCG,57TAG,65TAG} gene, the purified protein Ub-(11CbzK, 14CbzK, 57p-I-Phe, 65p-I-Phe) yielded tryptic peptides corresponding to the site-specific incorporation of CbzK into the 11 TCG and 14TCG codons (C) as well as p-I-Phe into the 57TAG (D) and 65TAG codons (E).

(F) For triple distinct ncAA incorporation into the Ub_{9TAG,11TCG,14TCA} gene, the purified protein Ub-(9p-I-Phe, 11CbzK, 14BocK) yielded tryptic peptides corresponding to the site-specific incorporation of p-I-Phe into the 9TAG codon, CbzK into the 11TCG codon, and BocK into the 14TCA codon.

FIG. 14 (S9). Multiple examples of encoding three distinct non-canonical amino acids into a single protein in Syn61Δ3(ev4).

(A) Chemical structures of the 8 non-canonical amino acids (ncAAs) used to demonstrate codon reassignment and triple ncAA incorporations in Syn61. The numbers indicate the identity of the ncAAs provided in (B).

(B) Seven examples of encoding three distinct ncAAs into a single protein in Syn61Δ3(ev4). Cells were transformed with plasmids encoding a His₆-tagged Ub_{9TAG,11TCG,14TCA} (or wt Ub) reporter plus three mutually orthogonal aaRS/tRNA pairs, and expression of the reporter was induced by addition of L-arabinose in the absence (−) or presence (+) of ncAAs. Full-length reporter expression was detected from an equal number of cells, normalized by OD₆₀₀ spectrophotometric measurements, by western blot with an anti-His₆ antibody. For expression of wt Ub and incorporation of CbzK, p-I-Phe and BocK/AllocK/CypK/AlkK (lanes 1-6), cells contained pairs 1R26PylRS(CbzK)/AlvtRNA^{ΔNPyl(8)}_{CGA}, AfTyrRS(p-I-Phe)/AftRNA^{Tyr(A01)}_{CUA} and MmPylRS/MmtRNA^{Pyl}_{UGA}. For incorporation of AllocK, 3-Nitro-Tyr and CbzK (lanes 7 and 8), cells contained pairs MmPylRS/MmtRNA^{Pyl}_{CGA}, MjTyrRS(3-Nitro-Tyr)/MjtRNA^{Tyr}_{CUA} (from ref (39)) and 1R26PylRS(CbzK)/AlvtRNA^{ΔNPyl(8)}_{UGA}. For incorporation of p-Az-Phe, CbzK and AllocK/AlkK (lanes 9-11), cells contained pairs MmPylRS/MmtRNA^{Pyl}_{CGA}, MjTyrRS(p-Az-Phe)/MjtRNA^{Tyr}_{CUA} (from ref (40)) and 1R26PylRS(CbzK)/AlvtRNA^{ΔNPYl(8)}_{UGA}. In all cases, expression of Ub_{9TAG,11TCG,14TCA} was dependent on provision of the three cognate ncAAs.

(C) The inventors purified Ub-His₆ reporters with Ni²⁺-NTA beads in Syn61Δ3(ev4) (see Methods) and performed ESI-MS analyses of the 7 different combinations of unnatural amino acids detailed in (B). Each trace corresponds to a different ubiquitin species, bearing the designated ncAAs at the positions indicated by the numbers. Ub(9p-I-Phe, 11CbzK, 14BocK) theoretical mass: 9820.97 Da; observed mass: 9820.8 Da—the −100 Da peak corresponds to loss of the tertbutoxycarbonyl in BocK. Ub(9p-I-Phe, 11CbzK, 14AllocK) theoretical mass: 9804.94 Da; observed mass: 9805.0 Da. Ub(9p-I-Phe, 11CbzK, 14CypK) theoretical mass: 9830.97 Da; observed mass: 9830.8 Da. Ub(9p-I-Phe, 11CbzK, 14AlkK) theoretical mass: 9802.88 Da; observed mass: 9803.0 Da. Ub(9(3-Nitro-Tyr), 11AllocK, 14CbzK) theoretical mass: 9740.09 Da; observed mass: 9740.0 Da. Ub(9p-Az-Phe, 11AllocK, 14CbzK) theoretical mass: 9720.09 Da; observed mass: 9720.2 Da. Ub(9p-Az-Phe, 11AlkK, 14CbzK) theoretical mass: 9717.99 Da; observed mass: 9718.6 Da. The ESI-MS data was collected once. See Fig. S10 for further confirmation of triple ncAA incorporation in response to the target codons. See Data file S4 for yields.

FIG. 15 (S10). MS-MS spectra from six additional triple distinct non-canonical amino acid (ncAA) combinations confirm the site-specific incorporation of the intended three ncAAs into Ub_{9TAG,11TCG,14TCA} reporters.

(A) To encode three distinct ncAA incorporations into the Ub_{9TAG,11TCG,14TCA} gene, the inventors expressed three orthogonal aaRS/tRNA pairs and added the corresponding three ncAAs to Syn61Δ3(ev4) cells. Ubiquitin containing ncAAs was purified by Ni²⁺-NTA and subjected to LC/MS-MS analyses following trypsin digestion to confirm the site-specific incorporation of distinct ncAAs into distinct TCG, TCA, and TAG codons (see Methods for full details). To encode the triple ncAA combination of p-I-Phe, CbzK, and Allock in response to TAG, TCG, and TCA codons, respectively, the same aaRS/tRNA pairs were used as in FIG. 4D. The purified protein Ub-(9p-IPhe, 11CbzK, 14AllocK) yielded tryptic peptides corresponding to the site-specific incorporation of p-I-Phe into the 9TAG codon, CbzK into the 11TCG codon, and AllocK into the 14TCA codon.

(B) For the triple ncAA combination incorporating p-I-Phe, CbzK, and CypK in response to the TAG, TCG, and TCA codons, respectively, the same aaRS/tRNA pairs were used as in FIG. 4D. The purified protein Ub-(9p-I-Phe, 11CbzK, 14CypK) yielded a tryptic peptide with the correct parent ion mass containing residues 9(p-I-Phe), 11(CbzK), and 14(CypK). The fragmentation pattern of this parent ion confirmed the site-specific incorporation of CbzK into the 11TCG codon and CypK into the 14TCA codon, though there were not enough b- and y-series ions to localize p-I-Phe to position 9.

(C) For the triple ncAA combination incorporating p-I-Phe, CbzK, and AlkK in response to the TAG, TCG, and TCA codons, respectively, the same aaRS/tRNA pairs were used as in FIG. 4D. The purified protein Ub-(9p-I-Phe, 11 CbzK, 14AlkK) yielded tryptic peptides corresponding to the site-specific incorporation of p-I-Phe into the 9TAG codon, CbzK into the 11TCG codon, and AlkK into the 14TCA codon.

(D) For the triple ncAA combination incorporating 3-Nitro-Tyr, AllocK, and CbzK in response to the TAG, TCG, and TCA codons, respectively, the inventors used the orthogonal aaRS/tRNA pairs MmPylRS/MmtRNA^{Pyl}_{CGA}, MjTyrRS(3-Nitro-Tyr)/MjtRNA^{Tyr}_{CUA} (from ref (39)), and 1R26PylRS(CbzK)/AlvtRNA^{ΔNPyl(8)}_{UGA}. The purified protein Ub-(9(3-Nitro-Tyr), 11AllocK, 14CbzK) yielded tryptic peptides corresponding to the site-specific incorporation of 3-Nitro-Tyr into the 9TAG codon, AllocK into the 11TCG codon, and CbzK into the 14TCA codon.

(E) For the triple ncAA combination incorporating p-Az-Phe, AllocK, and CbzK in response to the TAG, TCG, and TCA codons, respectively, the inventors used the orthogonal aaRS/tRNA pairs MmPylRS/MmtRNA^{Pyl}_{CGA}, MjTyrRS(p-Az-Phe)/MjtRNA^{Tyr}_{CUA} (from ref (40)), and 1R26PylRS(CbzK)/AlvtRNA^{ΔNPyl(8)}_{UGA}. The purified protein Ub-(9p-Az-Phe, 11AllocK, 14CbzK) yielded tryptic peptides corresponding to the site-specific incorporation of p-Az-Phe into the 9TAG codon, AllocK into the 11TCG codon, and CbzK into the 14TCA codon.

(F) For the triple ncAA combination incorporating p-Az-Phe, AlkK, and CbzK in response to the TAG, TCG, and TCA codons, respectively, the same orthogonal aaRS/tRNA pairs were used as in panel (E). The purified protein Ub-(9p-Az-Phe, 11 AllocK, 14CbzK) yielded tryptic peptides corresponding to the site-specific incorporation of p-Az-Phe into the 9TAG codon, AlkK into the 11TCG codon, and CbzK into the 14TCA codon.

FIG. 16 (S11). Encoded cellular synthesis of non-canonical elementary steps and heterotetramers.

(A) Elementary steps required for the synthesis of heteropolymer sequences made of BocK and p-I-Phe, encoded as insertions at position 3 of GFP. BocK (monomer A) and p-I-Phe (monomer B) are directed to TCG and TAG codons respectively, using pairs MmPylRS/MmtRNA^{Pyl}_{CGA} and AfTyrRS(p-I-Phe)/AftRNA^{Tyr(A01)}_{CUA} as in reassignment scheme 2 (r.s.2) in FIG. 5B. Polymerization and sfGFP expression were dependent on the addition of ncAAs to the medium.

(B) Encoding elementary steps made of CbzK and p-I-Phe, directed to TCG and TAG respectively using pairs 1R26PylRS(CbzK)/AlvtRNA^{ΔNPyl(8)}_{CGA} and AfTyrRS(p-I-Phe)/AftRNA^{Tyr(A01)}_{CUA} as in reassignment scheme 3 (r.s.3) in FIG. 5B.

(C) Encoding elementary steps made of AllocK and CbzK, directed to TCG and TAG respectively using pairs MmPylRS/MmtRNA^{Pyl}_{CGA} and 1R26PylRS(CbzK)/AlvtRNA^{ΔNPyl(8)}_{CUA} as in reassignment scheme 1 (r.s.1) in FIG. 5B.

(D) Schematic of genetic encoding of heterotetramer sequences with TCG and TAG codons, as in FIG. 5.

(E) Encoding tetramers made of BocK and p-I-Phe, directed to TCG and TAG respectively using pairs MmPylRS/MmtRNA^{Pyl}_{CGA} and AfTyrRS(p-I-Phe)/AftRNA^{Tyr(A01)}_{CUA} as in reassignment scheme 2 (r.s.2) in FIG. 5B.

(F) Encoding tetramers made of CbzK and p-I-Phe, directed to TCG and TAG respectively using pairs 1R26PylRS(CbzK)/AlvtRNA^{ΔNPyl(8)}_{CGA} and AfTyrRS(p-I-Phe)/AftRNA^{Tyr(A01)}_{CUA} as in reassignment scheme 3 (r.s.3) in FIG. 5B.

(G) Encoding tetramers made of AllocK and CbzK, directed to TCG and TAG respectively using pairs MmPylRS/MmtRNA^{Pyl}_{CGA} and 1R26PylRS(CbzK)/AlvtRNA^{ΔNPyl(8)}_{CUA} as in reassignment scheme 1 (r.s.1) in FIG. 5B.

FIG. 17 (S12). Encoded cellular synthesis of a non-canonical heterooctamer.

(A) Encoding a heterooctamer made of AllocK and CbzK, directed to TCG and TAG respectively using pairs MmPylRS/MmtRNA^{Pyl}_{CGA} and 1R26PylRS(CbzK)/AlvtRNA^{ΔNPyl(8)}_{CUA} as in reassignment scheme 1 (r.s.1) in FIG. 5B, synthesized as an insertion at position 3 of sfGFP.

(B) Polymerization of the encoded sequence composed of AllocK and CbzK, and the resulting sfGFP-His6 expression were dependent on the addition of both ncAAs to the medium.

(C) Electrospray ionization (ESI) MS of purified sfGFP-(3AllocK, 4CbzK, 5AllocK, 6CbzK, 7AllocK, 8CbzK, 9AllocK, 10CbzK) containing the indicated set of eight contiguous ncAAs. Expected mass after loss of N-terminal methionine and acetylation: 29608.28 Da; observed mass: 29607.60 Da. The ESI-MS data was collected twice.

FIG. 18 (S13). Excision of linear and cyclic non-canonical heteropolymers.

The heteropolymer is encoded and expressed in between His₆-SUMO and a GyrA intein (41)-CBD fusion, and the addition of cysteine after SUMO enables self-cyclisation. In a single step, the peptide is excised or cyclized through addition of Ulp1 and DTT. The Ulp1 rapidly removes the N-terminal SUMO, while the C-terminal GyrA intein is cleaved through transthioesterification by DTT or the N-terminal cysteine. The liberated heteropolymers can be purified from the reaction on a C18 column or, if insoluble, can be separated by centrifugation and extracted with hot, acidic methanol. The cyclisation is irreversible through the formation of a peptide bond by an N→S acyl shift, while the DTT thioester hydrolyses.

FIG. 19. RF1-mediated (ΔRF1) resistance against lambda phage is reversed upon KO of ssrA. On the contrary, lambda phage resistance in triple knockout (Δ3) cells is not reduced by ΔssrA.

FIG. 20. Provides a sequence alignment of various aaRS. “*” indicates residues found in all aligned aaRS sequences, “:” indicates residues within strong similarity between the aligned sequences, and “.” indicates residues within some similarity between the aligned sequences. Mm is Methanosarcina mazei; Mb is Methanosarcina barkeri; 1R26 is Methanomethylophilus sp. 1R26; Lum 1 is Methanomassiliicoccus luminyensis 1; Nitro is Nitrososphaeria archaeon; Tron is Methanonatronarchaeum thermophilum; Gemm is Gemmatimonadetes bacterium; PGA8 is Peptostreptococcaceae bacterium pGA-8; I2 is Desulfosporosinus sp. I2; Clos is Clostridiales bacterium; D121 is a Deltaproteobacteria bacterium; and D416 is another Deltaproteobacteria.

### DETAILED DESCRIPTION

The inventors demonstrate herein that it is possible to remove a first endogenous tRNA and a second endogenous tRNA from a prokaryotic cell, e.g. by deletion of the endogenous genes, to result in a viable cell.

Thus, in a first aspect of the invention, there is provided a prokaryotic cell wherein: the prokaryotic cell does not express a first endogenous tRNA and a second endogenous tRNA; and the prokaryotic cell comprises a genome wherein a first type of sense codon and a second type of sense codon have been recoded such that the first endogenous tRNA and the second endogenous tRNA are dispensable.

An endogenous tRNA is considered to be not expressed if the endogenous tRNA is not present in a form that would allow it to decode its cognate codon(s). Thus, an endogenous tRNA may be removed using any manner that would prevent the production of a functional form of the endogenous tRNA within the prokaryotic cell. For instance, the endogenous gene may be deleted or a portion of the gene may be deleted to prevent expression. Regulatory sequences may be deleted or altered to prevent expression. Alternatively, nonsense, frameshift, or missense mutations may prevent expression of the tRNA in a functional form.

The removals of the endogenous tRNAs are performed in prokaryotic cells wherein the genomes have been recoded to remove occurrences of particular types of sense codon. “Recoding” as used herein, is the replacement of an occurrence of a type of codon with a different codon, such that the occurrence of the codon is removed from the genome. The recoded sense codon may be replaced with a synonymous codon to result in different codon usage without changing the encoded polypeptide. Alternatively, the sense codon may be replaced with a non-synonymous codon, for instance if the alteration in the sequence of the encoded polypeptide does not affect viability. The deleted endogenous tRNAs are those that are dispensable in light of the recoding. “Dispensable” as used herein, means not required for viability of the prokaryotic cell.

Viable prokaryotic cells are cells that are capable of being metabolically active. In a particular embodiment, the viable prokaryotic cell of the invention may be capable of growth when cultured in an appropriate media and under appropriate conditions for the particular species or strain. Such prokaryotic cells may be referred to as capable of being cultured. As an example, if the prokaryotic cell is a bacterial cell such as E. coli, the assessment of viability may be performed by culturing said bacteria in a medium comprising LB medium, or on an agar comprising LB agar, at 37° C. The medium or agar may be supplemented with 2% glucose. Growth of the bacteria may be monitored using standard approaches, such as measurement of the OD₆₀₀. Alternative approaches, or approaches adapted to particular prokaryotic cells, bacteria strains, bacterial species, or in light of the inclusion of marker genes, are known to the skilled person.

A tRNA that decodes one or more sense codons that have been replaced (or deleted) may be deleted and the prokaryotic cell will remain viable if the tRNA decodes only the one or more sense codons that have been replaced (or deleted); or alternatively if the tRNA decodes one or more sense codons that have been replaced (or deleted) and one or more sense codons that have not been replaced (or deleted), if the tRNA is dispensable for the one or more sense codons that have not been replaced (or deleted) (i.e. the one or remaining sense codons which the tRNA decodes are decoded by one or more alternative tRNAs). For example, if the genome of the prokaryotic cell lacks TCA sense codons, serT, encoding tRNA^{Ser}_{UGA}, may be deleted and/or if the genome lacks TCG sense codons, serU, encoding tRNA^{Ser}_{CGA}, may be deleted. Thus, in an embodiment, the prokaryotic cell expresses neither tRNA^{Ser}_{UGA} nor tRNA^{Ser}_{CGA}.

The number of occurrences of the first and second type of sense codon that are recoded is adequate to enable the removal of the cognate tRNAs corresponding to said sense codons while maintaining viability of the prokaryotic cell. For example, this may be achieved by removing all of the occurrences of the first and second type of sense codon from the essential genes. A gene is “essential”, as used herein, if the product of the gene is required for viability of the prokaryotic cell. For instance, if the prevention of expression of a functional form of a protein encoded by a gene would result in non-viability of the prokaryotic cell, then the gene is considered essential. In particular, a gene is considered essential if a “blank” codon (i.e. a codon for which the cell contains no corresponding tRNA or release factor) within the gene results in a loss of cell viability. Therefore, in an embodiment, all of the genes of the prokaryotic cell for which a blank codon could not be tolerated without a loss of viability are recoded, but genes that are able to tolerate blank codons may not be recoded. Thus, the skilled person can assess whether all of the essential genes have been recoded by assessing whether a cognate tRNA is dispensable.

In a particular embodiment, there is provided a prokaryotic cell, wherein: the prokaryotic cell does not express a first endogenous tRNA and a second endogenous tRNA; the essential genes of the prokaryotic cell do not contain occurrences of a first type of sense codon, and the first endogenous tRNA is a cognate tRNA for the first type of sense codon; and the essential genes of the prokaryotic cell do not contain occurrences of a second type of sense codon, and the second endogenous tRNA is a cognate tRNA for the second type of sense codon.

In particular embodiments, the genome comprises 100 or more, 200 or more, or 300 or more essential genes with no occurrences of the first and/or second type of sense codon. For instance, all or substantially all of the essential genes in the genome may comprise no occurrences of the first and/or second type of sense codon.

In some embodiments, the essential genes may be selected from one or more of the list consisting of: ribF, ispA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, tsf, pyrH, frr, dxr, ispU, cdsA, yaeL, yaeT, lpxD, fabZ, lpxA, lpxB, dnaE, accA, tilS, proS, yafF, hemB, secD, secF, ribD, ribE, thiL, dxs, ispA, dnaX, adk, hemH, lpxH, cysS, folD, entD, mrdB, mrdA, nadD, holA, rlpB, leuS, lnt, glnS, fldA, cydA, infA, cydC, ftsK, lolA, serS, rpsA, msbA, lpxK, kdsB, mukF, mukE, mukB, asnS, fabA, mviN, rne, fabD, fabG, acpP, tmk, holB, lolC, olD, lolE, purB, minE, minD, pth, prsA, ispE, lolB, hemA, prfA, prmC, kdsA, topA, ribA, fabI, tyrS, ribC, ydiL, pheT, pheS, rplT, infC, thrS, nadE, gapA, yeaZ, aspS, argS, pgsA, yejM, metG, folE, yejM, gyrA, nrdA, nrdB, folC, accD, fabB, gltX, ligA, zipA, dapE, dapA, der, hisS, ispG, suhB, tadA, acpS, era, rnc, lepB, rpoE, pssA, yfiO, rplS, trmD, rpsP, ffh, grpE, csrA, ispF, ispD, ftsB, eno, pyrG, chpR, lgt, fbaA, pgk, yqgD, metK, yqgF, plsC, ygiT, parE, ribB, cca, ygjD, tdcF, yraL, yhbV, infB, nusA, ftsH, obgE, rpmA, rplU, ispB, murA, yrbB, yrbK, yhbN, rpsI, rplM, degS, mreD, mreC, mreB, accB, accC, yrdC, def fint, rplQ, rpoA, rpsD, rpsK, rpsM, secY, rplO, rpmD, rpsE, rplR, rplF, rpsH, rpsN, rplE, rplX, rplN, rpsQ, rpmC, rplP, rpsC, rplV, rpsS, rplB, rplW, rplD, rplC, rpsJ, fusA, rpsG, rpsL, trpS, yrfF, asd, rpoH, ftsX ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, glmS, glmU, wzyE, hemD, hemC, yigP, ubiB, ubiD, hemG, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, and dnaC.

In particular, the essential genes may be selected from one or more of the list consisting of: ribF, ispA, ispH, dapB, folA, imp, yabQ, lpxC, secM, secA, can, folK, hemL, yadR, dapD, map, rpsB, tsf, pyrH, frr, dxr, ispU, cdsA, yaeL, yaeT, lpxD, fabZ, lpxA, lpxB, dnaE, accA, tilS, proS, yafF, hemB, secD, secF, ribD, ribE, thiL, dxs, ispA, dnaX, adk, hemH, lpxH, cysS, folD, entD, mrdB, mrdA, nadD, holA, rlpB, leuS, lnt, glnS, fldA, cydA, infA, cydC, ftsK, lolA, serS, rpsA, msbA, bpxK, kdsB, mukF, mukE, mukB, asnS, fabA, mviN, rne, fabD, fabG, acpP, tmk, holB, iolC, bolD, bolE, purB, minE, minD, pth, prsA, ispE, lolB, hemA, prfA, prmC, kdsA, topA, ribA, fabI, tyrS, ribC, ydiL, pheT, pheS, rplT, infC, thrS, nadE, gapA, yeaZ, aspS, argS, pgsA, yejM, metG, folE, yejM, gyrA, nrdA, nrdB, folC, accD, fabB, gltX, higA, zipA, dapE, dapA, der, hisS, ispG, suhB, tadA, acpS, era, rnc, lepB, rpoE, pssA, yfiO, rplS, trmD, rpsP, ffh, grpE, csrA, ispF, ispD, ftsB, eno, pyrG, chpR, lgt, fbaA, pgk, yqgD, metK, yqgF, plsC, ygiT, parE, ribB, cca, ygjD, tdcF, yraL, yhbV, infB, nusA, ftsH, obgE, rpmA, rplU, ispB, murA, yrbB, yrbK, yhbN, rpsI, rplM, degS, mreD, mreC, mreB, accB, accC, yrdC, def fint, rplQ, rpoA, rpsD, rpsK, rpsM, secY, rplO, rpmD, rpsE, rplR, rplF, rpsH, rpsN, rplE, rplX rplN, rpsQ, rpmC, rplP, rpsC, rplV, rpsS, rplB, rplW, rplD, rplC, rpsJ, fusA, rpsG, rpsL, trpS, yrfF, asd, rpoH, ftsX ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, glmS, glmU, wzyE, hemD, hemC, yigP, ubiB, ubiD, hemG, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, and dnaC.

In other embodiments, the prokaryotic cell of the present invention may comprise a genome comprising 5 or fewer occurrences of the first and/or second type of sense codon. The genome may be derived from a parent genome and may comprise less than 10%, 5%, 2%, 1%, 0.5%, 0.1% of the occurrences of the first and/or second type of sense codon, relative to the parent genome. The genome may comprise 100 or more, 200 or more, or 1000 or more genes with no occurrences of the first and/or second type of sense codon. In particular, all or substantially all the genes in the genome may have no occurrences of the first and/or second type of sense codon.

Thus, in a particular embodiment, there is provided a prokaryotic cell, wherein: the prokaryotic cell does not express a first endogenous tRNA and a second endogenous tRNA; the genome of the prokaryotic cell comprises 5, 4, 3, 2, 1, or no occurrences of a first type of sense codon, and the first endogenous tRNA is a cognate tRNA for the first type of sense codon; and the genome of the prokaryotic cell comprises 5, 4, 3, 2, 1, or no occurrences of a second type of sense codon, and the second endogenous tRNA is a cognate tRNA for the second type of sense codon.

In a particular embodiment, the genome comprises no occurrences of the first type of sense codon and no occurrences of the second type of sense codon.

The genome may be derived from a parent genome and comprise 5 or fewer (e.g. 5, 4, 3, 2, 1), or no occurrences of native sense codons of the first and/or second type. In a particular embodiment, the genome is derived from a parent genome and comprises no occurrences of native sense codons of the first and the second type.

In some embodiments the genome comprises 100 or more, 200 or more, 300 or more, 400 or more, 500 or more, 600 or more, 700 or more, 800 or more, 900 or more, 1000 or more, 1500 or more, or 2000 or more recoded genes. In some embodiments the genes are those for which there is evidence of translation and/or of the predicted protein product. For example, the genome may comprise 100 or more, 200 or more, 300 or more, 400 or more, 500 or more 600 or more, 700 or more, 800 or more, 900 or more, 1000 or more, 1500 or more, or 2000 or more recoded genes for which there is evidence of translation and/or of the predicted protein product.

In an embodiment, all annotated open reading frames within the genome have no occurrences of the sense codons of the first and the second type. The prokaryotic cell of the invention may be a bacterial cell, preferably E. coli, and the genome of the E. coli may contain no occurrences of a first and a second type of sense codon as annotated in GenBank accession number CP040347.1.

In a particular embodiment, the protein-encoding genes have no occurrences of the sense codons of the first and the second type. In particular embodiments, no proteins are translated from any of the remaining occurrences of the first and/or second type of sense codon and/or genes comprising the remaining occurrences of the first and/or second type of sense codons are putative or are non-coding genes. In some embodiments the translation of the genes comprising the remaining occurrences of the first and/or second type of sense codons is reduced and/or prevented (e.g. the genes may comprise stop codons in the 5′ sequence).

Any remaining occurrences of the sense codons may be necessary to ensure that the genome is viable. For example, one or more, in particular all, of the remaining occurrences of the first and/or second type of sense codons in the genome may be present in the regulatory elements of essential genes; and/or one or more, in particular all, of the remaining occurrences of the first and/or second type of sense codons may be in genes in which there is no evidence for translation or the predicted protein product (i.e. putative or non-coding genes).

As used herein, a “sense codon” is a nucleotide triplet that codes for an amino acid. Thus, sense codons may be identified in a genome by gene prediction, i.e. by identifying regions of the genome that code for proteins (i.e. genes) and the corresponding open reading frames (ORFs). Typically, genomes naturally comprise 61 sense codons: GCT, GCC, GCA, GCG, CGT, CGC, CGA, CGG, AGA, AGG, AAT, AAC, GAT, GAC, TGT, TGC, CAA, CAG, GAA, GAG, GGT, GGC, GGA, GGG, CAT, CAC, ATT, ATC, ATA, TTA, TTG, CTT, CTC, CTA, CTG, AAA, AAG, ATG, TTT, TTC, CCT, CCC, CCA, CCG, TCT, TCC, TCA, TCG, AGT, AGC, ACT, ACC, ACA, ACG, TGG, TAT, TAC, GTT, GTC, GTA, and GTG (read from 5′ to 3′ on the coding strand of DNA). The standard genetic code encodes the 20 canonical amino acids using the 61 triplet codons. 18 of the 20 amino acids are encoded by more than one synonymous codon. The first or second type of sense codon may be native sense codons, i.e. sense codons which are present in the parent genome.

The 61 sense codons in DNA are transcribed into corresponding mRNA and subsequently decoded by one or more tRNAs. tRNAs carry an amino acid to a ribosome as directed by the sense codons in the mRNA. The tRNAs can recognise one or more sense codons via a complementary anticodon. A sequence of sense codons is subsequently translated into a polypeptide (i.e. a sequence of amino acids). Codon and anticodon interactions in the E. coli genome are shown in FIG. 17 of WO2020/229592 (incorporated herein by reference).

The genome wide removal of the first and/or second type of sense codon, but not other sense codons, enables cognate tRNAs corresponding to said first or second type of sense codons to be deleted without removing the ability to decode the sense codons remaining in the genome.

Aminoacyl-tRNA synthetases for serine do not recognise the anticodons of their cognate tRNAs. This may facilitate the assignment of codons to new amino acids through the introduction of tRNAs bearing cognate anticodons that do not direct mis-aminoacylation by endogenous synthetases. Thus, the recoded sense codons may be selected from: TCG, TCA, TCT, TCC, AGT, or AGC. In a particular embodiment, the first and second type of sense codon are TCA and TCG.

To achieve removal of sense codons they may be replaced with synonymous sense codons. This is preferable to ensure that the encoded protein sequence is not changed. For instance, the prokaryotic cell may have a genome wherein 90% or more, 95% or more, 98% or more, 99% or more, 99.5% or more, 99.6% or more, 99.7% or more, 99.8% or more, 99.9% or more, or 100% of the occurrences of the first or second type of sense codons in the parent genome is replaced with synonymous sense codons. The person skilled in the art is able to deduce suitable synonymous sense codon replacements. For example, in E. coli, typically TCG, TCA, TCT, TCC, AGT and AGC all encode serine.

In some embodiments, the replacement is a defined replacement, i.e. one sense codon is replaced with a single synonymous sense codon. Preferably, 90% or more, 95% or more, 98% or more, 99% or more, 99.5% or more, 99.6% or more, 99.7% or more, 99.8% or more, 99.9% or more, or 100% of the occurrences of the first or second type of sense codon in the parent genome are is replaced with a defined (i.e. single) synonymous sense codon.

For example, the defined replacement may be: TCG replaced with any one of TCT, TCC, AGT, or AGC; or TCA replaced with any one of TCT, TCC, AGT, or AGC.

In particular, the replacements are selected from one or more of: TCG to either AGT or AGC; or TCA to either AGT or AGC. In a particular embodiment, TCG is replaced with AGC and TCA is replaced with AGT.

Preferably, none of these codon replacements affect ribosomal binding sites (AGGAGG), which are highly conserved regulatory sequences in E. coli. The selected codon replacements may be tested on a small test region (e.g. a 20 kb region of the genome rich in both essential target genes and target codons) to assess viability. If the codon replacements are not viable on the small test region they may be disregarded.

When replacement of sense codons in the parent genome with defined replacement synonymous sense codons does not result in a viable prokaryotic cell, alternative replacement synonymous sense codons may be used. For instance, 99.9% of the occurrences of the first and/or second type of sense codon in the parent genome may be replaced with a defined (i.e. single) synonymous sense codon, and the remaining 0.1% with alternative synonymous sense codons. For example, 99.9% of the occurrences of TCG may be replaced with AGC and 0.1% replaced with TCT, TCC, AGT or AGC; and/or 99.9% of the occurrences of TCA may be replaced with AGT and 0.1% replaced with TCT, TCC, AGT or AGC.

In some instances, a particular occurrence of a sense codon may not be replaceable with any of the potential synonymous sense codons without affecting viability. To retain viability, the sense codon may be replaced with a non-synonymous sense codon that does not affect viability. For instance, 99.9% of the occurrences of the first and/or second type of sense codon in the parent genome may be replaced with a defined (i.e. single) synonymous sense codon, and the remaining 0.1% with alternative non-synonymous sense codons.

The inventors have additionally demonstrated that it is possible to remove an endogenous release factor from a prokaryotic cell of the invention while retaining viability, such that the resultant cell does not express at least two endogenous tRNAs and does not express at least one endogenous release factor. Thus, in an embodiment, the prokaryotic cell does not express a first endogenous release factor; and a first type of stop codon has been recoded within the genome of the prokaryotic cell such that the first endogenous release factor is dispensable.

The removal of the first endogenous release factor may performed in prokaryotic cells wherein the genomes have been recoded to remove occurrences of a first type of stop codon. Optionally the removed stop codons are replaced with synonymous codons. The deleted endogenous release factor is the factor that is dispensable in light of the recoding.

In a particular embodiment, there is provided a prokaryotic cell, wherein the prokaryotic cell does not express a first endogenous tRNA, a second endogenous tRNA, and a first endogenous release factor; and wherein the genome of the prokaryotic cell has been recoded to remove a plurality of the sense codons for which the first and second endogenous tRNAs are cognate, and to remove a plurality of the stop codon for which the first endogenous release factor is cognate.

An endogenous release factor is considered to be not expressed if the endogenous release factor is not present in a form that would allow it to decode its cognate codon(s). Thus, an endogenous release factor may be removed using any manner that would prevent the production of a functional form of the endogenous release factor within the prokaryotic cell. For instance, the endogenous gene may be deleted or a portion of the gene may be deleted to prevent expression. Regulatory sequences may be deleted or altered to prevent expression. Alternatively, nonsense, frameshift, or missense mutations may prevent expression of the release factor in a functional form.

As used herein, a “stop codon” is a nucleotide triplet that codes for termination of translation into proteins. Typically, genomes naturally comprise 3 stop codons: TAA (“ochre”), TGA (“opal” or “umber”) and TAG (“amber”).

The number of occurrences of the first type of stop codon that are removed is adequate to enable the removal of the cognate release factor corresponding to said stop codons while maintaining viability of the prokaryotic cell.

Thus, in some embodiments, the essential genes of the prokaryotic cell do not contain occurrences of the first type of stop codon. The essential genes may be any as discussed herein, particularly those discussed in relation to the removal of the first or second type of sense codon. In particular embodiments, the genome comprises 100 or more, 200 or more, or 300 or more essential genes with no occurrences of the first type of stop codon. For instance, all or substantially all of the essential genes in the genome may comprise no occurrences of the first type of stop codon.

For example, the genome may comprise 100 or more, 200 or more, or 300 or more essential genes with no occurrences of the first type of sense codon, the second type of sense codon, and the first type of stop codon. In particular, all or substantially all of the essential genes in the genome may comprise no occurrences of the first type of sense codon, the second type of sense codon, and the first type of stop codon.

In some embodiments, the genome comprises 10 or fewer, 5 or fewer, or no occurrences of the first type of stop codon. Such as 5, 4, 3, 2, 1, or no instances of the first type of stop codon.

In a particular embodiment, wherein the first type of stop codon is TAG and the first endogenous release factor is RF-1. In such embodiments, there may be 10 or fewer, 5 or fewer, or no occurrences of the amber stop codon (TAG). In other examples, 90% or more, 95% or more, 98% or more, 99% or more, or all of the occurrences of TAG in the parent genome are replaced with TAA (the ochre stop codon). In particular embodiments, the genome comprises no occurrences of the amber stop codon (TAG), optionally wherein all of the occurrences of TAG in the parent genome are replaced with TAA (the ochre stop codon).

In an embodiment, all annotated open reading frames within the genome have no occurrences of the first type of stop codon. The prokaryotic cell of the invention may be a bacterial cell, preferably E. coli, and the genome of the E. coli may contain no occurrences of first type of stop codon as annotated in GenBank accession number CP040347.1.

In a particular embodiment, the protein-encoding genes have no occurrences of the first type of stop codon. In particular embodiments, no proteins are translated from any of the remaining occurrences of the first type of stop codon and/or genes comprising the remaining occurrences of the first type of stop codon are putative or are non-coding genes. In some embodiments the translation of the genes comprising the remaining occurrences of the first type of stop codon is reduced and/or prevented (e.g. the genes may comprise stop codons in the 5′ sequence).

Any remaining occurrences of the first type of stop codon may be necessary to ensure that the genome is viable. For example, one or more, in particular all, of the remaining occurrences of the first type of stop codon in the genome may be present in the regulatory elements of essential genes; and/or one or more, in particular all, of the remaining occurrences of the first type of stop codon may be in genes in which there is no evidence for translation or the predicted protein product (i.e. putative or non-coding genes).

Accordingly, in some embodiments the genome comprises no occurrences of a first and a second type of sense codon, and no occurrences of one stop codon, preferably the amber stop codon (TAG). In particular embodiments the genome comprises no occurrences of the sense codons TCG and TCA, and no occurrences of the amber stop codon (TAG), optionally wherein TCG, TCA and TAG in the parent genome are replaced with synonymous codons, for example 99.9% or more of the occurrences of TCG in the parent genome are replaced with AGC, 99.9% or more of the occurrences of TCA in the parent genome are replaced with AGT and all of the occurrences of TAG in the parent genome are replaced with TAA.

In a particular embodiment, the genome of the prokaryotic cell has been recoded such that the sense codon TCG has been replaced with AGC, the sense codon TCA has been replaced with AGT, and the stop codon TAG has been replaced with TAA, and wherein sufficient numbers of said codons have been recoded such that two cognate tRNAs and a cognate release factor are dispensable.

In a particular embodiment, the prokaryotic cell of the invention is an E. coli cell that does not express tRNA^{Ser}_{UGA}, tRNA^{Ser}_{CGA}, or RF-1, occurrences of the sense codon TCA have been recoded such that tRNA^{Ser}_{UGA} is dispensable (e.g. occurrences of TCA in essential genes of the parent strain have been replaced with AGT), occurrences of the sense codon TCG have been recoded such that tRNA^{Ser}_{CGA} is dispensable (e.g. occurrences of TCG in essential genes of the parent strain have been replaced with AGC), and occurrences of the stop codon TAG have been recoded such that RF-1 is dispensable (e.g. occurrences of TAG in essential genes of the parent strain have been replaced with TAA). The prokaryotic cell may also have been modified to include first, second, and third orthogonal aminoacyl-tRNA synthetase-tRNA pairs capable of decoding the TCA, TCG, and TAG codons and incorporating non-canonical amino acids into such sites during polymer formation.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to the sequence provided in GenBank accession number CP040347.1, and wherein the genome has been further altered such that tRNA^{Ser}_{UGA} and tRNA^{Ser}_{CGA} are not functionally expressed (for instance, by deleting serT and serU). The genome may have been even further altered such that RF-1 is not functionally expressed (for instance, by deleting prfA). An E. coli strain comprising a genome according to GenBank accession number CP040347.1 is referred to as Syn61 WT in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 3, and wherein the genome has been further altered such that tRNA^{Ser}_{UGA} and tRNA^{Ser}_{CGA} are not functionally expressed (for instance, by deleting serT and serU). The genome may have been even further altered such that RF-1 is not functionally expressed (for instance, by deleting prfA). An E. coli strain comprising a genome according to SEQ ID NO: 3 is referred to as Syn61(ev1) in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 4, and wherein the genome has been further altered such that tRNA^{Ser}_{UGA} and tRNA^{Ser}_{CGA} are not functionally expressed (for instance, by deleting serT and serU. The genome may have been even further altered such that RF-1 is not functionally expressed (for instance, by deleting prfA). An E. coli strain comprising a genome according to SEQ ID NO: 4 is referred to as Syn61(ev2) in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 5. An E. coli strain comprising a genome according to SEQ ID NO: 5 is referred to as Syn61Δ3 in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 6. An E. coli strain comprising a genome according to SEQ ID NO: 6 is referred to as Syn61Δ3(ev3) in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 7. An E. coli strain comprising a genome according to SEQ ID NO: 7 is referred to as Syn61Δ3(ev4) in the Examples disclosed herein.

In some embodiments the genome of the prokaryotic cell of the invention comprises a polynucleotide sequence which is at least 80%, 85%, 90%, 95%, 98%, 99%, 99.5%, 99.8%, 99.9%, or 100% identical to SEQ ID NO: 8 (also provided as GenBank accession number CP071799.1). An E. coli strain comprising a genome according to SEQ ID NO: 8 is referred to as Syn61Δ3(ev5) in the Examples disclosed herein.

There is provided herein a prokaryotic cell comprising a genome which is at least 80%, 85%, 90%, 95%, 98%, 99%, 99.5%, 99.8%, 99.9%, or 100% identical to any one of: SEQ ID NO: 3, SEQ ID NO: 4, SEQ ID NO: 5, SEQ ID NO: 6, SEQ ID NO: 7, and SEQ ID NO: 8. The prokaryotic cell may be a bacterium, for instance E. coli. In some embodiments, the calculation of the sequence identity percentage excludes any sequence that has been inserted to enable the expression of an orthogonal aminoacyl-tRNA synthetase-tRNA pair or multiple orthogonal pairs. The calculation of sequence identity percentage may further exclude any exogenous sequences that have been further introduced into the genome. The genomes with less than 100% identity may comprise the recoded sense and stop codons as disclosed herein, hence in such embodiments the variation does not reintroduce the first type of sense codon, second type of sense codon, or first type of stop codon.

The inventors have identified that prokaryotic cells with recoded genomes or which lack a first and/or a second endogenous tRNA may have growth defects, unless specific steps are taking during the production of said prokaryotic cells. These steps are described herein.

The inventors further noted that some methods of inducing mutagenesis, for instance those involving the use of a mutagenesis plasmid, are not functional in the prokaryotic cells of the invention. The inventors have overcome this issue through the provision of specifically adapted reagents.

Thus, in a particular embodiment, the growth rate of the prokaryotic cell is faster than the growth rate of a reference prokaryotic cell of a parental strain.

The growth rates may be compared by preparing a cell culture of the prokaryotic cell of the invention, and a cell culture of the reference prokaryotic cell, and comparing the rate of change of the optical density of the culture medium. In some embodiments, the prokaryotic cell is a bacterial cell, such as E. coli, and the growth rates are compared by determined the doubling time at 37° C., 25° C. or 42° C. in LB media. The doubling time of the prokaryotic cell of the invention may be 1.1, 1.2, 1.3, 1.4, 1.5, 1.6, 1.7, 1.8, 1.9, 2, or 3 times faster than the doubling time of the reference prokaryotic cell.

A “parental strain” is a strain that existed or was created as part of the development of the prokaryotic cell of the invention. Hence, the prokaryotic cell of the invention is derived, directly or indirectly, from the parental strain. A parental strain may also be referred to as a “progenitor strain”.

The parental strain may be the prokaryotic cell strain obtained upon recoding the genome to remove particular types of sense and/stop codons, but before further steps are taken. The parental strain may comprise a genome wherein a first type of sense codon, a second type of sense codon, and a first type of stop codon have been recoded in the same manner as for a prokaryotic cell of the invention, and wherein the parental strain contains all endogenous tRNAs and release factors. For example, the parental strain may be the strain obtained upon recoding the genome to remove or reduce the number of TCG, TCA, and TAG codons. In a particular embodiment, the parental strain may be Syn61 or may comprise the genome of Syn61 (i.e. the sequence in GenBank accession number CP040347.1).

The parental strain may be the prokaryotic cell strain obtained upon removal of a first endogenous tRNA, a second endogenous tRNA, and/or a release factor, but before further steps are taken. For example, the parental strain may be the strain obtained upon removal of tRNA^{Ser}_{UGA}, tRNA^{Ser}_{CGA}, and RF-1. In a particular embodiment, the parental strain may be Syn61Δ3 or may comprise the genome of Syn61Δ3 (SEQ ID NO: 5).

The inventors provide experimental data herein demonstrating that the prokaryotic cell of the invention are resistant to phage. While it had been previously hypothesised that removing cellular tRNAs would provide phage resistance, the inventors have found that the removal of a first and a second endogenous tRNA provides complete resistance. This level of phage resistance is beyond that which would be predicted from the additive effect of removing these two tRNAs. Without being bound to a particular theory, the inventors note that endogenous tRNAs overlap in the codons that they are able to decode, and that some degree of read-through may be possible even where the tRNA is not normally known to be associated with the codon. As such, the removal of the first and the second endogenous tRNA synergises to provide a completely resistant prokaryotic cell.

Further to the preceding paragraph, the inventors provide data herein that demonstrate that the prokaryotic cells of the invention are more resistant to the accumulation of mutations that could lead to the rescue of viral propagation. For instance, the inventors noted that knocking out the gene ssrA in other strains, which have at least some phage resistance, can rescue viral propagation. In contrast, the knock out of ssrA from prokaryotic cells of the invention does not rescue viral propagation (FIG. 19). As such, the prokaryotic cells of the invention demonstrate surprisingly robust phage resistance, even if further mutations are accumulated.

Hence, in an embodiment, the prokaryotic cell of the invention is resistant to phage infection. The phage may be any phage containing the first type of sense codon, the second type of sense codon, and/or the first type of stop codon in its genome. For example, the prokaryotic cell of the invention may be resistant to T4 phage, T6 phage, T7 phage, λ phage, or P1vir phage. In some embodiments, prokaryotic cells of the invention are resistant to all five of these examples.

The prokaryotic cell of the invention may be resistant to horizontal gene transfer. For instance, horizontal transfer of the F plasmid.

The prokaryotic cell of the invention may be more resistant to infection or horizontal gene transfer than a reference prokaryotic cell which is of a parental strain. The parental strain may be any as described herein.

In some embodiments, prokaryotic cell of the invention are completely resistant to phage. The prokaryotic cell may be completely resistant to T4 phage, T6 phage, T7 phage, λ phage, and P1vir phage. The term “completely resistant”, as used herein, means that the growth rate of a culture of the prokaryotic cell of the invention is substantially or completely unaffected by the inclusion of a relevant phage in said culture. A control may be the inclusion of the same amount of killed phage. A relevant phage is a phage that would be capable of infecting a parental strain of the prokaryotic cell.

In particular embodiments, the prokaryotic cell of the invention is a bacterial cell. The bacterial cell may be of any species suitable for heterologous protein production, in particular the production of polypeptides comprising one or more non-canonical amino acids (for instance those described by Ferrer-Miralles, N. and Villaverde, A., 2013. Microbial Cell Factories, 12:113). Suitable bacterial host cells include: escherichia (e.g. Escherichia coli), caulobacteria (e.g. Caulobacter crescentus), phototrophic bacteria (e.g. Rodhobacter sphaeroides), cold adapted bacteria (e.g. Pseudoalteromonas haloplanktis, Shewanella sp. strain Ac10), pseudomonads (e.g. Pseudomonas fluorescens, Pseudomonas putida, Pseudomonas aeruginosa), halophilic bacteria (e.g. Halomonas elongate, Chromohalobacter salexigens), streptomycetes (e.g. Streptomyces lividans, Streptomyces griseus), nocardia (e.g. Nocardia lactamdurans), mycobacteria (e.g. Mycobacterium smegmatis), coryneform bacteria (e.g. Corynebacterium glutamicum, Corynebacterium ammoniagenes, Brevibacterium lactofermentum), bacilli (e.g. Bacillus subtilis, Bacillus brevis, Bacillus megaterium, Bacillus licheniformis, Bacillus amyloliquefaciens), vibrio bacteria (e.g. Vibrio cholera, Vibrio natriegens), and lactic acid bacteria (e.g. Lactococcus lactis, Lactobacillus plantarum, Lactobacillus casei, Lactobacillus reuteri, Lactobacillus gasseri). In some embodiments the bacterium is gram-negative bacterium.

In particular embodiments, the bacterium is an Escherichia coli, Salmonella enterica, or Shigella dysenteriae. More preferably, the cell is an E. coli. Suitable E. coli cells include K-12, MG1655, BL21, BL21(DE3), AD494, Origami, HMS174, BLR(DE3), HMS174(DE3), Tuner(DE3), Origami2(DE3), Rosetta2(DE3), Lemo21(DE3), NiCo21(DE3), T7 Express, SHuffle Express, C41(DE3), C43(DE3), and m15 pREP4 or derivatives thereof (Rosano, G. L. and Ceccarelli, E. A., 2014. Frontiers in microbiology, 5, p. 172). In particular, the cell may be MG1655 or BL21, or a derivative thereof. MG1655 is considered as the wild type strain of E coli. The GenBank ID of genomic sequence of this strain is U00096. BL21 is widely available commercially. For example, it can be purchased from New England BioLabs with catalog number C2530H.

The prokaryotic cell may contain a genome which is derived from the same species or strain, or may be derived from a different species. For example, if the prokaryotic cell is E. coli the genome may be an E. coli genome.

### Prokaryotic Cells Comprising Orthogonal Aminoacyl-tRNA Synthetase-tRNA Pairs

The inventors provide experimental data herein demonstrating that a first orthogonal aminoacyl-tRNA synthetase and a first orthogonal tRNA, which form an orthogonal pair, may be introduced into a prokaryotic cell of the invention to replace an endogenous tRNA. The first orthogonal tRNA may decode a type of sense codon which has been removed from the genome, for instance the first type of sense codon may have been removed from the genome in any manner disclosed herein. The first orthogonal aminoacyl-tRNA synthetase specifically aminoacylates the orthogonal cognate tRNA with an amino acid, which may be a non-canonical amino acid. Hence, the prokaryotic cell may contain a system wherein an endogenous tRNA has been replaced with a first orthogonal tRNA, such that a first type of sense codon is repurposed to code for a non-canonical amino acid.

In addition, the inventors provide experimental data herein demonstrating that a first orthogonal aminoacyl-tRNA synthetase-tRNA pair and a second orthogonal aminoacyl-tRNA synthetase-tRNA pair may be introduced into a prokaryotic cell of the invention. The first tRNA may decode one of the sense codons removed from the genome, and the second tRNA may decode the other sense codon. The orthogonal aminoacyl-tRNA synthetases specifically aminoacylate their orthogonal cognate tRNA with an amino acid, which may be a non-canonical amino acid. Hence, the prokaryotic cell may contain a system wherein two or more sense codons have been repurposed to code for non-canonical amino acids.

In cells wherein a stop codon has been recoded, a third orthogonal aminoacyl-tRNA synthetase-tRNA pair may be introduced into the prokaryotic cell, allowing the recoded stop codon to be used to code for a third non-canonical amino acid. Hence, the prokaryotic cell may contain a system wherein two or more sense codons and one or more stop codons have been repurposed to code for a first, second, and third non-canonical amino acid. In a particular embodiment, the prokaryotic cell contains a system wherein TCA, TCG, and TAG encode a first, second, and third non-canonical amino acid. The first, second, and third non-canonical amino may be different from each other.

Thus, the orthogonal aminoacyl-tRNA synthetase (aaRS)-tRNA pairs may be capable of directing the incorporation of non-canonical amino acids into polymers using the protein synthesis apparatus of the prokaryotic cell. The orthogonal synthetases do not recognize endogenous tRNAs, and specifically aminoacylate an orthogonal cognate tRNA (which is not an efficient substrate for endogenous synthetases) with the non-canonical amino acids provided to (or synthesised by) the cell (Chin, J. W., 2017. Nature, 550(7674), 53-60).

In an embodiment, there is provided a prokaryotic cell wherein: the prokaryotic cell does not express a first endogenous tRNA, the prokaryotic cell comprises a genome wherein a first type of sense codon has been recoded such that the first endogenous tRNA is dispensable, the prokaryotic cell expresses a first orthogonal aminoacyl-tRNA synthetase and a first orthogonal tRNA, the first orthogonal aminoacyl-tRNA synthetase and the first orthogonal tRNA form a first orthogonal aminoacyl-tRNA synthetase-tRNA pair, and the first orthogonal tRNA is capable of decoding the first type of sense codon.

In another embodiment, there is provided a prokaryotic cell wherein: the prokaryotic cell does not express a first endogenous tRNA and a second endogenous tRNA, the prokaryotic cell comprises a genome wherein a first type of sense codon and a second type of sense codon have been recoded such that the first endogenous tRNA and the second endogenous tRNA are dispensable,
the prokaryotic cell expresses a first orthogonal aminoacyl-tRNA synthetase and a first orthogonal tRNA, the first orthogonal aminoacyl-tRNA synthetase and the first orthogonal tRNA form a first orthogonal aminoacyl-tRNA synthetase-tRNA pair, the first orthogonal tRNA is capable of decoding the first type of sense codon, and
the prokaryotic cell expresses a second orthogonal aminoacyl-tRNA synthetase and a second orthogonal tRNA, the second orthogonal aminoacyl-tRNA synthetase and the second orthogonal tRNA form a second orthogonal aminoacyl-tRNA synthetase-tRNA pair, the second orthogonal tRNA is capable of decoding the second type of sense codon.

In yet another embodiment, there is provided a prokaryotic cell, wherein:
the prokaryotic cell does not express a first endogenous tRNA, a second endogenous tRNA, and a first endogenous release factor,
the prokaryotic cell comprises a genome wherein a first type of sense codon and a second type of sense codon have been recoded such that the first endogenous tRNA and the second endogenous tRNA are dispensable, and a first type of stop codon has been recoded such that the first endogenous release factor is dispensable,
the prokaryotic cell expresses a first orthogonal aminoacyl-tRNA synthetase and a first orthogonal tRNA, the first orthogonal aminoacyl-tRNA synthetase and the first orthogonal tRNA form a first orthogonal aminoacyl-tRNA synthetase-tRNA pair, the first orthogonal tRNA is capable of decoding the first type of sense codon;
the prokaryotic cell expresses a second orthogonal aminoacyl-tRNA synthetase and a second orthogonal tRNA, the second orthogonal aminoacyl-tRNA synthetase and the second orthogonal tRNA form a second orthogonal aminoacyl-tRNA synthetase-tRNA pair, the second orthogonal tRNA is capable of decoding the second type of sense codon; and
the prokaryotic cell expresses a third orthogonal aminoacyl-tRNA synthetase and a third orthogonal tRNA,
the third orthogonal aminoacyl-tRNA synthetase and the third orthogonal tRNA form a third orthogonal aminoacyl-tRNA synthetase-tRNA pair, and
the third orthogonal tRNA is capable of decoding the first type of stop codon.

The aminoacyl-tRNA synthetases used herein may be varied. Although specific tRNA synthetase sequences may have been used in the examples, the invention is not intended to be confined only to those examples. In principle any aminoacyl-tRNA synthetase which provides a tRNA charging (aminoacylation) function can be employed. For example the tRNA synthetase may be from any suitable species such as from archaea, for example from Methanosarcina—such as Alethanosarcina barkeri MS; Methanosarcina barkeri str. Fusaro; Methanosarcina mazei G01; Methanosarcina acetivorans C2A; Methanosarcina thermophila; or Methanococcoides—such as Methanococcoides burtonii. Alternatively the tRNA synthetase may be from bacteria, for example from Desulfitobacterium—such as Desulfitobacterium hafniense DCB-2; Desulfitobacterium hafniense Y51; Desulfitobacterium hafniense PCP1; or Desulfotomaculum acetoxidans DSM 771.

The aminoacyl-tRNA synthetase may be a pyrrolysyl tRNA synthetase (PylRS). The PylRS may be a wild-type or a genetically engineered PylRS. Genetically engineered PylRS has been described, for example, by Neumann et al. (Nat Chem Biol 4:232, 2008) and by Yanagisawa et al. (Chem Biol 2008, 15:1187), in EP2192185A1, and in WO2016/066995 (each incorporated herein by reference). Suitably, a genetically engineered tRNA synthetase gene is selected that increases the incorporation efficiency of non-canonical amino acid(s). The PylRS may be Methanosarcina barkeri (MbPylRS) or Methanosarcina mazei (MmPylRS).

The tRNA used herein may be varied. Although specific tRNAs may have been used in the examples, the invention is not intended to be confined only to those examples. In principle, any tRNA can be used provided that it is compatible with the selected tRNA synthetase.

The tRNA may be from any suitable species such as from archea, for example from Methanosarcina—such as Methanosarcina barkeri MS; Methanosarcina barkeri str. Fusaro; Methanosarcina mazei. G01; Methanosarcina acetivorans C2A; Methanosarcina thermophila; or Methanococcoides—such as Methanococcoides burtonii. Alternatively the tRNA may be from bacteria, for example from Desulfitobacterium—such as Desulfitobacterium hafniense DCB-2; Desulfitobacterium hafniense Y51; Desulfitobacterium hafniense PCP1; or Desulfotomaculum acetoxidans DSM 771.

The tRNA gene can be a wild type tRNA gene or it may be a mutated tRNA gene. Suitably, a mutated tRNA gene is selected that increases the incorporation efficiency of unnatural amino acid(s). In one embodiment, the mutated tRNA gene is a U25C variant of PylT as described in Biochemistry (2013) 52, 10 (incorporated herein by reference).

In one embodiment, the mutated tRNA gene is an Opt variant of PylT as described in Fan et al. (Nucleic Acids Research doi:10.1093/nar/gkv800) (incorporated herein by reference herein).

In one embodiment, the mutated tRNA gene has both the U25C and the Opt variants of PylT, i.e. in this embodiment the tRNA, such as the PylT tRNA_{CUA} gene, comprises both the U25C and the Opt mutations.

In one embodiment, the sequence encoding the tRNA is the pyrrolysine tRNA (PylT) gene from Methanosarcina mazei pyrrolysine which encodes tRNAPyl.

The aminoacyl-tRNA synthetase and tRNA pair may be as disclosed in, or adapted from those disclosed in, Cervettini et al. (Rapid discovery and evolution of orthogonal aminoacyl-tRNA synthetase-tRNA pairs, Nature Biotechnology, Vol 38, 990 August 2020, P 989-999) or Dunkelmann et al. (Engineered triply orthogonal pyrrolysyl-tRNA synthetase/tRNA pairs enable the genetic encoding of three distinct non-canonical amino acids, Nature Chemistry, Vol 12, June 2020 P 535-544). Each of these documents is incorporated by reference.

As mentioned in the background section, the aaRS, tRNA, and codon preferably function together and are orthogonal to each endogenous amino acid, aaRS and group of isoacceptor tRNAs and their cognate group of codons. In addition, there is the potential for interactions between components of the first, second, and third ncAA:aaRS:tRNA:codon sets, and these are preferably also minimised.

As such, the identification of multiple engineered mutually orthogonal aaRS/tRNA pairs that recognise distinct codons and incorporate distinct non-canonical amino acids, and can be used in combination, provides a valuable contribution to the art. The inventors provide herein novel aaRS and tRNA pairs, and also provide new combinations of said pairs that are demonstrated herein to function in combination.

Thus, in an aspect of the invention there is provided the aaRS Methanomethylophilus sp. 1R26 PylRS (1R26PylRS). 1R26PylRS may have the following sequence:

(SEQ ID NO: 9)
MAEHFTDAQIQRLREYGNGTYKDMEFADVSAREKAFTKLMSDASRDNES
ALKGMIAHPARQGLSRLMNDIADALVADGFIEVRTPIIISKDALAKMTI
TPDKPLFKQVFWIDDKRALRPMLAPSLYTVMRSLRDHTDGPVKIFEMGS
CFRKESHSGMHLEEFTMLNLVDMGPAGDATESLKKYIGIVMKAAGLPDY
QLVHEESDVYKETIDVEINGQEVCSAAVGPHYLDAAHDVHEPWAGAGFG
LERLLTIRQGYSTVMKGGASTTYLNGAKMD.

In an embodiment, the synthetase is a variant of 1R26PylRS further comprising mutations necessary to allow the recognition of, or to alter the specificity for, a non-canonical amino acid. In particular embodiments, the aaRS may be according to SEQ ID NO: 9, comprising any one or a combination of substitutions at the following residues: L121, L125, Y126, M129, N166, V168, Y206, A223. Mutation at the aforementioned residues may be used to generate variants of 1R26PylRS with altered selectivity to non-canonical amino acids.

The synthetase may be 1R26PylRS(CbzK). 1R26PylRS(CbzK) may be a mutant of 1R26PylRS comprising the following mutations: i) Y126G and M129L, or ii) M129A and Y206F. 1R26PylRS(CbzK) is suitable for directing the incorporation of NE-(carbobenzyloxy)-1-lysine (CbzK). 1R26PylRS(CbzK) may according to SEQ ID NO: 10 or 11.

The synthetase may be 1R26PylRS(AllocK). 1R26PylRS(AllocK) may be a mutant of 1R26PylRS comprising the mutations N166Q and Y206F, and is suitable for directing the incorporation of 6-N-Alloc-L-lysine (AllocK). 1R26PylRS(AllocK) may according to SEQ ID NO: 12.

The synthetase may be 1R26PylRS(Bta). 1R26PylRS(Bta) may be a mutant of 1R26PylRS comprising the mutations N166S, V168M, Y206F, and A223G, and is suitable for directing the incorporation of 3-Benzothienyl-L-alanine (Bta). 1R26PylRS(Bta) may according to SEQ ID NO: 13.

The synthetase may be 1R26PylRS(NMH). 1R26PylRS(NMH) may be a mutant of 1R26PylRS comprising the mutations L121M, L125I, Y126F, M129A, V168F, and is suitable for directing the incorporation of 3-N-Methyl-L-histidine (NMH). 1R26PylRS(NMH) may according to SEQ ID NO: 14.

In an embodiment, the aaRS is at least 50%, 60%, 70%, 80%, 90%, 95%, 99%, or 100% similar or identical to any one of SEQ ID NOs: 9-14. The aaRS may be at least 50%, 60%, 70%, 80%, 90%, 95%, 99%, or 100% identical to SEQ ID NO: 9, comprising any one of the following groups of mutations: i) L121L, L125L, Y126G, M129L, N166N, V168V, Y206Y, A223A; ii) L121L, L125L, Y126Y, M129A, N166N, V168V, Y206F, A223A; iii) L121L, L125L, Y126Y, M129M, N166Q, V168V, Y206F, A223A; iv) L121L, L125L, Y126Y, M129M, N166S, V168M, Y206F, A223G; and v) L121M, L125I, Y126F, M129A, N166N, V168F, Y206Y, A223A.

FIG. 20 provides a sequence alignment where “*” indicates residues found in all aligned aaRS sequences, “:” indicates residues within strong similarity between the aligned sequences, and “.” indicates residues within some similarity between the aligned sequences. The aaRS may be at least 50%, 60%, 70%, 80%, 90%, 95%, 99%, or 100% identical to any one of SEQ ID NOs: 9-14, wherein the variation is found only in residues not marked with: i) “*”; ii) “*” or “:”, or iii) “*” or “:”, or “.”.

1R26PylRS, and its variants and derivatives, may paired with the tRNA Methanomethylophilus alvus (Alv) tRNA^{ΔNPyl(8)}.

Thus, in another aspect of the invention, there is provided AlvtRNA^{NPyl(8)}. AlvtRNA^{ΔNPyl(8)} may have the following sequence:

(SEQ ID NO: 61)
GGGGGACGGTCCGGCGACCAGCGGGTCTNNNAAACCTAGCcttgCGGGGT
TCGACaCCCCGGTCTCTCGCCA,

wherein the anticodon is the “NNN” triplet.

In a particular embodiment, AlvtRNA^{ΔNPyl(8)} may have the following sequence:

(SEQ ID NO: 15)
GGGGGACGGTCCGGCGACCAGCGGGTCTCTAAAACCTAGCcttgCGGGGT
TCGACaCCCCGGTCTCTCGCCA.

AlvtRNA^{ΔNPyl(8)} may be modified to include any anticodon. AlvtRNA^{ΔNPyl(8)}_{CGA} is exemplified herein, but the invention need not be limited in this manner. The tRNA may be at least 80%, 90%, 95%, 99%, or 100% identical to SEQ ID NO: 15 or 61.

In an aspect of the invention, there is provided a host cell comprising any 1R26PylRS, or variant or derivative thereof, disclosed herein and any AlvtRNA^{ΔNPyl(8)}, or variant or derivative thereof, disclosed herein, wherein the 1R26PylRS and AlvtRNA^{ΔNPyl(8)} form an orthogonal aminoacyl-tRNA synthetase-tRNA pair. The host cell may be any suitable for heterologous protein production, in particular the production of polypeptides comprising one or more non-canonical amino acids. The host cell may be a prokaryotic or a eukaryotic cell. The host cell may be a bacterial cell, such as E. coli. The host cell may be a eukaryotic cell, such as a mammalian or insect cell line. The host cell may be a human cell. The host cell may be any bacterium of the invention as disclosed herein.

1R26PylRS and AlvtRNA^{ΔNPyl(8)} are also provided herein in the isolated forms. A molecule is considered to be “isolated”, as used herein, when it is not found in its natural environment (i.e. when it is not expressed as a wild type protein in the cells in which it occurs naturally). A molecule may be isolated and found as part of another composition (e.g. if expressed by a host cell which would not naturally express the molecule).

Another aaRS disclosed herein is the Archaeoglobus fulgidus tyrosyl-tRNA synthetase (AfTyrRS). AfTyrRS is suitable for use in the prokaryotic cell of the present invention. AfTyrRS may have the following sequence:

(SEQ ID NO: 16)
MDITEKLRLITRNAEEVVTEEELRQLIETKEKPRAYVGYEPSGEIHLGHM
MTVQKLMDLQEAGFEIIVLLADIHAYLNEKGTFEEIAEVADYNKKVFIAL
GLDESRAKFVLGSEYQLSRDYVLDVLKMARITTLNRARRSMDEVSRRKED
PMVSQMIYPLMQALDIAHLGVDLAVGGIDQRKIHMLARENLPRLGYSSPV
CLHTPILVGLDGQKMSSSKGNYISVRDPPEEVERKIRKAYCPAGVVEENP
ILDIAKYHILPRFGKIVVERDAKFGGDVEYASFEELAEDFKSGQLHPLDL
KIAVAKYLNMLLEDARKRLGVSV

In an embodiment, the synthetase is a variant of AfTyrRS further comprising mutations necessary to allow the recognition of, or to alter the specificity for, a non-canonical amino acid. In particular embodiments, the aaRS may be according to SEQ ID NO: 16, comprising any one or a combination of substitutions at the following residues: Y36, L69, H74, Q116, D165, I166, or N190. Mutation at the aforementioned residues may be used to generate variants of AfTyrRS with altered selectivity to non-canonical amino acids.

In another embodiment, the synthetase may be AfTyrRS(p-I-Phe). AfTyrRS(p-I-Phe) may be a mutant of AfTyrRS comprising the following mutations: Y36I, L69M, H74L, Q116E, D165T, and I166G, and is suitable for directing the incorporation of p-I-Phe. AfTyrRS(p-I-Phe) may be according to SEQ ID NO: 17.

In another embodiment, the synthetase may be AfTyrRS(p-Az-Phe). AfTyrRS(p-Az-Phe) may be a mutant of AfTyrRS comprising the following mutations: Y36T, H74L, Q116E, D165T, I166G, and N190K, and is suitable for directing the incorporation of p-Az-Phe. AfTyrRS(p-Az-Phe) may be according to SEQ ID NO: 18.

In another embodiment, the synthetase may be AfTyrRS(o-Methyl-Tyrosine). AfTyrRS(o-Methyl-Tyrosine) may be a mutant of AfTyrRS comprising the following mutations: Y36I, H74L, Q116E, D165T, I166G, and is suitable for directing the incorporation of o-Methyl-Tyrosine. AfTyrRS(o-Methyl-Tyrosine) may be according to SEQ ID NO: 19.

In an embodiment, the aaRS may be at least 50%, 60%, 70%, 80%, 90%, 95%, 99%, or 100% similar or identical to any one of SEQ ID NOs: 16 to 19. The aaRS may be at least 50%, 60%, 70%, 80%, 90%, 95%, 99%, or 100% identical to SEQ ID NO: 16, comprising any one of the following groups: i) Y36I, L69M, H74L, Q116E, D165T, I166G, N190N; ii) Y36T, L69L, H74L, Q116E, D165T, I166G, N190K; and iii) Y36I, L69L, H74L, Q116E, D165T, I166G, N190N.

AfTyrRS, and its variants and derivatives, may be paired with the tRNA Af-tRNA^{Tyr(A01)}_{YYY}. Af-tRNA^{Tyr(A01)}_{YYY} may have the following sequence:

(SEQ ID NO: 62)
CCCGCCCTAGCTCAGAGGTAGAGCGTGCTTCTNNNAAAAGCATGGTCCCC
GGTTCAAATCCTGGGGGCGGGACCA,

wherein the anticodon is the “NNN” triplet.

In a particular embodiment, Af-tRNA^{Tyr(A01)} may have the following sequence:

(SEQ ID NO: 20)
CCCGCCCTAGCTCAGAGGTAGAGCGTGCTTCTctaAAAAGCATGGTCCCC
GGTTCAAATCCTGGGGGCGGGACCA

Af-tRNA^{Tyr(A01)}_{YYY} may be modified to include any anticodon. Af-tRNA^{Tyr(A01)}_{CUA} is exemplified herein, but the invention need not be limited in this manner. The tRNA may be at least 80%, 90%, 95%, 99%, or 100% identical to SEQ ID NO: 20 or 62.

Another aaRS disclosed herein is MmPylRS. MmPylRS is an aaRS suitable for use in the prokaryotic cell of the present invention. MmPylRS may have the following sequence:

(SEQ ID NO: 21)
MDKKPLNTLISATGLWMSRTGTIHKIKHHEVSRSKIYIEMACGDHLVVNN
SRSSRTARALRHHKYRKTCKRCRVSDEDLNKFLTKANEDQTSVKVKVVSA
PTRTKKAMPKSVARAPKPLENTEAAQAQPSGSKFSPAIPVSTQESVSVPA
SVSTSISSISTGATASALVKGNTNPITSMSAPVQASAPALTKSQTDRLEV
LLNPKDEISLNSGKPFRELESELLSRRKKDLQQIYAEERENYLGKLEREI
TRFFVDRGFLEIKSPILIPLEYIERMGIDNDTELSKQIFRVDKNFCLRPM
LAPNLYNYLRKLDRALPDPIKIFEIGPCYRKESDGKEHLEEFTMLNFCQM
GSGCTRENLESIITDFLNHLGIDFKIVGDSCMVYGDTLDVMHGDLELSSA
VVGPIPLDREWGIDKPWIGAGFGLERLLKVKHDFKNIKRAARSESYYNGI
STNL.

In an embodiment, the synthetase is a variant of MmPylRS further comprising mutations necessary to allow the recognition of, or alter the specificity for, a non-canonical amino acid. In particular embodiments, the aaRS may be according to SEQ ID NO: 21, comprising any one or a combination of substitutions at the following residues: L301, A302, L305, Y306, L309, N346, C348, Y384, V401, or W417. Mutation at the aforementioned residues may be used to generate variants of MmPylRS with altered selectivity to non-canonical amino acids.

The synthetase may be MmPylRS(Bpa). MmPylRS(Bpa) may be a mutant of MmPylRS comprising the mutations A302T, N346T, C348T, and W417C, and is suitable for directing the incorporation of p-Benzoyl-L-phenylalanine (Bpa). MmPylRS(Bpa) may be according to SEQ ID NO: 22.

The synthetase may be MmPylRS(Bta). MmPylRS(Bta) may be a mutant of MmPylRS comprising the mutations N346S, C348M, Y384F, and V401G, and is suitable for directing the incorporation of 3-Benzothienyl-L-alanine (Bta). MmPylRS(Bta) may be according to SEQ ID NO: 23.

The synthetase may be MmPylRS(CbzK). MmPylRS(CbzK) may be a mutant of MmPylRS comprising the mutations: i) Y306G, or ii) L309A, C348V, Y384F, and is suitable for directing the incorporation of N6-Cbz-L-Lysine (CbzK). MmPylRS(CbzK) may be according to SEQ ID NO: 24 or SEQ ID NO: 25.

The synthetase may be MmPylRS(NMH). MmPylRS(NMH) may be a mutant of MmPylRS comprising the mutations L301M, L305I, Y306F, L309A, and C348F, and is suitable for directing the incorporation of 3-N-Methyl-L-histidine (NMH). MmPylRS(NMH) may be according to SEQ ID NO: 26.

In other embodiments, the MmPylRS may be adapted to be suitable for directing the incorporation of BocK, AllocK, p-I-Phe, CypK, or AlkK.

In an embodiment, the aaRS may be at least 50%, 60%, 70%, 80%, 90%, 95%, 99%, or 100% similar or identical to any one of SEQ ID NOs: 21 to 26. The aaRS may be at least 50%, 60%, 70%, 80%, 90%, 95%, 99%, or 100% identical to SEQ ID NO: 21, comprising any one of the following groups: i) L301L, A302T, L305L, Y306Y, L309L, N346T, C348T, Y384Y, V401V, W417C; ii) L301L, A302A, L305L, Y306Y, L309L, N346S, C348M, Y384F, V401G, W417W; iii) L301L, A302A, L305L, Y306G, L309L, N346N, C348C, Y384Y, V401V, W417W, iv) L301L, A302A, L305L, Y306Y, L309A, N346N, C348V, Y384F, V401V, W417W; and v) L301M, A302A, L305I, Y306F, L309A, N346N, C348F, Y384Y, V401V, W417W.

FIG. 20 provides a sequence alignment. The aaRS may be at least 50%, 60%, 70%, 80%, 90%, 95%, 99%, or 100% identical to any one of SEQ ID NOs: 21-26, wherein the variation is found only in residues not marked with: i) “*”; ii) “*” or “:”, or iii) or “*”, “:”, or “.”.

MmPylRS, and derivatives, may paired with the tRNA MmtRNA^{Pyl}_{YYY}. MmtRNA^{Pyl}_{YYY} may have the following sequence:

(SEQ ID NO: 63)
GGaAACCTGATCATGTAGATCGAATGGACTNNNAATCCGTTCAGCCGGGT
TAGATTCCCGGGGTTTCCGCCA,

wherein the anticodon is the “NNN” triplet.

In an embodiment, the MmtRNA^{Pyl} may have the following sequence:

(SEQ ID NO: 27)
GGaAACCTGATCATGTAGATCGAATGGACTCTAAATCCGTTCAGCCGGGT
TAGATTCCCGGGGTTTCCGCCA.

MmtRNA^{Pyl}_{YYY} may be modified to include any anticodon. MmtRNA^{Pyl}_{UGA} is exemplified herein, but the invention need not be limited in this manner. The tRNA may be at least 80%, 90%, 95%, 99%, or 100% identical to SEQ ID NO: 27 or 63.

Another aaRS disclosed herein is M jannaschii tyrosyl-tRNA synthetase (MjTyrRS). MjTyrRS is an aaRS suitable for use in the prokaryotic cell of the present invention. The MjTyrRS may be as disclosed in J. N. Beyer et al. (Journal of Molecular Biology 432, 4690-4704 (2020); incorporated herein by reference).

MjTyrRS may have the following sequence:

(SEQ ID NO: 28)
MDEFEMIKRNTSEIISEEELREVLKKDEKSAYIGFEPSGKIHLGHYLQIK
KMIDLQNAGFDIIILLADLHAYLNQKGELDEIRKIGDYNKKVFEAMGLKA
KYVYGSEFQLDKDYTLNVYRLALKTTLKRARRSMELIAREDENPKVAEVI
YPIMQVNDIHYLGVDVAVGGMEQRKIHMLARELLPKKVVCIHNPVLTGLD
GEGKMSSSKGNFIAVDDSPEEIRAKIKKAYCPAGVVEGNPIMEIAKYFLE
YPLTIKRPEKFGGDLTVNSYEELESLFKNKELHPMDLKNAVAEELIKILE
PIRKRL.

In an embodiment, the synthetase is a variant of MjTyrRS further comprising mutations necessary to allow the recognition of, or to alter the specificity for, a non-canonical amino acid. In particular embodiments, the aaRS may be according to SEQ ID NO: 28, comprising any one or a combination of substitutions at the following residues: Y32, L65, H70, F108, Q109, D158, I159, or L162. Mutation at the aforementioned residues may be used to generate variants of MjTyrRS with altered selectivity to non-canonical amino acids.

In another embodiment, the synthetase may be MjTyrRS(3-Nitro-Tyr). MjTyrRS(3-Nitro-Tyr) is a mutant of MjTyrRS comprising the mutations Y32H, H70T, D158H, I159A, and L162R. MjTyrRS(3-Nitro-Tyr) is suitable for directing the incorporation 3-Nitro-Tyr. MjTyrRS(3-Nitro-Tyr) may be according to SEQ ID NO: 29.

In another embodiment, the synthetase may be MjTyrRS(p-Az-Phe). MjTyrRS(p-Az-Phe) is a mutant of MjTyrRS comprising the mutations Y32L, L65V, F108W, Q109M, D158G, and I159A. MjTyrRS(p-Az-Phe) is suitable for directing the incorporation p-Az-Phe. MjTyrRS(p-Az-Phe) may be according to SEQ ID NO: 30.

In an embodiment, the aaRS may be at least 50%, 60%, 70%, 80%, 90%, 95%, 99%, or 100% similar or identical to any one of SEQ ID NOs: 28 to 30. The aaRS may be at least 50%, 60%, 70%, 80%, 90%, 95%. 99%. or 100% identical to SEQ ID NO: 28, comprising any one of the following groups: i) Y32H, L65L, H70T, F108F, Q109Q, D158H, I159A, L162R; and ii) Y32L, L65V, H70H, F108W, Q109M, D158G, I159A, L162L.

MjTyrRS, and derivatives, may paired with the tRNA MjtRNA^{Tyr}_{YYY}. The MjRNA^{Tyr}_{YYY} may be as disclosed in N. Beyer et al. (Journal of Molecular Biology 432, 4690-4704 (2020). MjtRNA^{Tyr}_{YYY} may have the following sequence:

(SEQ ID NO: 64)
CCGGCGGTAGTTCAGCAGGGCAGAACGGCGGACTNNNAATCCGCATGGCA
GGGGTTCAAATCCCCTCCGCCGGACCA,

wherein the anticodon is the “NNN” triplet.

In an embodiment, the MjtRNA^{Tyr} may have the following sequence:

(SEQ ID NO: 31)
CCGGCGGTAGTTCAGCAGGGCAGAACGGCGGACTCTAAATCCGCATGGCA
GGGGTTCAAATCCCCTCCGCCGGACCA.

MjtRNA^{Tyr}_{YYY} may be modified to include any anticodon. MjtRNA^{Tyr}_{CGA} is exemplified herein, but the invention need not be limited in this manner. The tRNA may be at least 80%, 90%, 95%, 99%, or 100% identical to SEQ ID NO: 31 or 64.

Another aaRS disclosed herein is Methanomassiliicoccus luminyensis 1 (Lum1) PylRS. Lum1PylRS is an aaRS suitable for use in the prokaryotic cell of the present invention. The Lum1PylRS may have the following sequence:

(SEQ ID NO: 32)
MDTRLTPAQAQRIREMGGTVDPSLAFSSEAERESAFQRISADLQGANLAK
IRRCAEAPERHPIGSLENTLACALAAKGFIEVKTPMMIPADGLVKMGIDE
SHPLWNQVFWVGPKKALRPMLAPNLYFLMRHLRRSVPAPLLLFEIGPCFR
KESRGSNHLEEFTMLNLVELAPQADATERLKEHIATVMNAVGLPYELVVE
GSEVYGTTIDVEVDGVELASGAVGPLPMDKPHGITEPWAGVGFGLERIAL
MRTKEQNIKKVGRSLVYVNGARIDI.

In an embodiment, the synthetase is a variant of Lum1PylRS further comprising mutations necessary to allow the recognition of, or to alter the specificity for, a non-canonical amino acid. In particular embodiments, the aaRS may be according to SEQ ID NO: 32, comprising any one or a combination of substitutions at the following residues: L121, L125, Y126, M129, and V168. Mutation at the aforementioned residues may be used to generate variants of Lum1PylRS with altered selectivity to non-canonical amino acids.

The synthetase may be Lum1PylRS(CbzK). Lum1PylRS(CbzK) may be a mutant of Lum1PylRS comprising the mutations Y126G and M129L, and is suitable for directing the incorporation of CbzK. Lum1PylRS(CbzK) may be according to SEQ ID NO: 33.

The synthetase may be Lum1PylRS(NMH). Lum1PylRS(NMH) may be a mutant of Lum1PylRS comprising the mutations L121M, L125I, Y126F, M129A, and V168F, and is suitable for directing the incorporation of 3-N-Methyl-L-histidine (NMH). Lum1PylRS(NMH) may be according to SEQ ID NO: 34.

In an embodiment, the aaRS may be at least 50%, 60%, 70%, 80%, 90%, 95%, 99%, or 100% similar or identical to any one of SEQ ID NOs: 32 to 34. The aaRS may be 50%, 60%, 70%, 80%, 90%, 95%, 99%, or 100% identical to SEQ ID NO: 32, comprising any one of the following groups: i) L121L, L125L, Y126G, M129L, V168V; and ii) L121M, L125I, Y126F, M129A, V168F.

FIG. 20 provides a sequence alignment. The aaRS may be at least 50%, 60%, 70%, 80%, 90%, 95%, 99%, or 100% identical to any one of SEQ ID NOs: 32-34, wherein the variation is found only in residues not marked with: i) “*”; ii) “*” or “:”, or iii) or “*”, “:”, or “.”.

Lum1PylRS, and derivatives, may paired with the M. intenstinalis tRNAs. Such tRNAs may have the following sequence:

(SEQ ID NO: 65)
GGCGAACTGGTCCGGGACCACCAGGCCTNNNAAGCCACGGTTAGCCGGGT
TCAACTCCCGGGTTCGTCGCCA,

wherein the anticodon is the “NNN” triplet.

In an embodiment, the M. intenstinalis tRNA may have the following sequence:

(SEQ ID NO: 66)
GGCGAACTGGTCCGGGACCACCAGGCCTctaAAGCCACGGTTAGCCGGGT
TCAACTCCCGGGTTCGTCGCCA

The M. intenstinalis tRNA may be modified to include any anticodon. The tRNA may be at least 80%, 90%, 95%, 99%, or 100% identical to SEQ ID NO: 65 or 66.

Another aaRS disclosed herein is NitroPylRS, which is an aaRS from Nitrososphaeria archaeon (NCBI protein accession is HHP52415.1). NitroPylRS is an aaRS suitable for use in the prokaryotic cell of the present invention. The NitroPylRS may have the following sequence:

(SEQ ID NO: 35)
MSKIRFTRGQIHRLIELGAEPTELERDFETEAERDKEFNKIAENLARKNL
KNIKDFLEQRRKPLVRVIEEKLRTTALRLGFSEVVTPIIIPRLFIKRMGI
DEGDPLWKQVMLIDDKRALRPMLAPNLYVLMAKLSNIVRPVKIFEIGPCF
RRETGGRYHLEEFTMFNMVELAPEGDPKERLLDYIDTIMRDIGLNYTISV
EPSNVYGETLDVVVNGIEVASAAIGPKPIDANWGVREPWIGVGFGVERLA
MLVGGYNSIARIAKSLSYLDGSTLSVIKLRW.

In an embodiment, the synthetase is a variant of NitroPylRS further comprising mutations necessary to allow the recognition of, or to alter the specificity for, a non-canonical amino acid.

The synthetase may be NitroPylRS (NMH). NitroPylRS(NMH) may be a mutant of NitroPylRS comprising the mutations L123M, L127I, Y128F, M131A, and V169F, and is suitable for directing the incorporation of NMH. NitroPylRS(NMH) may be according to SEQ ID NO: 36.

In an embodiment, the aaRS may be at least 50%, 60%, 70%, 80%, 90%, 95%, 99%, or 100% similar or identical to SEQ ID NO: 35 or SEQ ID NO: 36. The aaRS may be at least 50%, 60%, 70%, 80%, 90%, 95%, 99%, or 100% identical to SEQ ID NO: 36, comprising the following substitutions L123M, L127I, Y128F, M131A, and V169F.

FIG. 20 provides a sequence alignment. The aaRS may be at least 50%, 60%, 70%, 80%, 90%, 95%, 99%, or 100% identical to SEQ ID NO: 35 or SEQ ID NO: 36, wherein the variation is found only in residues not marked with: i) “*”; ii) “*” or “:”, or iii) or “*”, “:”, or “.”.

NitroPylRS, and derivatives, may paired with the a tRNA derived from Methanohalarchaeum thermophilum. Such tRNAs may have the following sequence:

(SEQ ID NO: 53)
GGGGGGCTGGTCGGGTGGCCAAGGGGGCTCTAAACCCTCGGTTGCCGGGT
TCAACTCCCGGGCTCCCCACCA.

SEQ ID NO: 53 may be modified to include any anticodon. The tRNA may be at least 80%, 90%, 95%, 99%, or 100% identical to SEQ ID NO: 53.

Another aaRS disclosed herein is ClosΔNTDPylRS, which is an aaRS from Clostridiales bacterium (accession no. NLY82529.1). ClosΔNTDPylRS is an aaRS suitable for use in the prokaryotic cell of the present invention. The ClosΔNTDPylRS may have the following sequence:

(SEQ ID NO: 37)
MENFTITQTERLKQLNCENDVLELEFEDSEARNSKFREIEIGRVKKGKEN
IKNLLKEKHITISDEVGNKLSDWLMSKDYTKVLTPTIISKDQLKAMTIDE
ENHLFSQVFWIDNNKCLRPMLAPNLYIVMRELKRITNEPVKIFEIGSCFR
KESQGARHMNEFTMLNMVELASVEDGKQLDTLKALAHEAMESLGVESYEL
VIEESAVYGSTLDIEIDGIEVASGSYGPHELDANWDIFDTWVGIGFGIER
LAMAINGGSTIKKYGRSINFIDGETMKL.

In an embodiment, the synthetase is a variant of ClosΔNTDPylRS further comprising mutations necessary to allow the recognition of, or to alter the specificity for, a non-canonical amino acid. In particular embodiments, the aaRS may be according to SEQ ID NO: 37, comprising any one or a combination of substitutions at the following residues: Y126, M129, Y208. Mutation at the aforementioned residues may be used to generate variants of ClosΔNTDPylRS with altered selectivity to non-canonical amino acids.

The synthetase may be ClosΔNTDPylRS(CbzK). ClosΔNTDPylRS(CbzK) may be a mutant of ClosΔNTDPylRS comprising the mutations: i) Y126G and M129L, or ii) M129A and Y208F, and is suitable for directing the incorporation of CbzK. ClosΔNTDPylRS(CbzK) may be according to SEQ ID NO: 38 or SEQ ID NO: 39.

In an embodiment, the aaRS may be at least 50%, 60%, 70%, 80%, 90%, 95%, 99%, or 100% similar or identical to any one of SEQ ID NOs: 37 to 39. The aaRS may be at least 50%, 60%, 70%, 80%, 90%, 95%, 99%, or 100% identical to SEQ ID NO: 37, comprising any one of the following groups: i) Y126G, M129L, and Y208Y, or ii) Y126Y, M129A, or Y208F.

FIG. 20 provides a sequence alignment. The aaRS may be at least 50%, 60%, 70%, 80%, 90%, 95%, 99%, or 100% identical to any one of SEQ ID NOs: 37 to 39, wherein the variation is found only in residues not marked with: i) “*”; ii) “*” or “:”, or iii) or “*”, “:”, or “.”.

ClosΔNTDPylRS, and its variants and derivatives, may be paired with a tRNA according to the following sequence:

(ClostRNA; SEQ ID NO: 56)
GGGGAACGGATCGGATGGATCACATAGACTCTAAATCTATGTAGCCGAGT
GAAACTCTCGGGTTCCTCGCCA

ClostRNA may be modified to include any anticodon. The tRNA may be at least 80%, 90%, 95%, 99%, or 100% identical to SEQ ID NO: 56.

Another aaRS disclosed herein is an aaRS derived from Methanonatronarchaeum thermophilum (TronΔNTDPylRS). TronΔNTDPylRS is an aaRS suitable for use in the prokaryotic cell of the present invention. The TronΔNTDPylRS may have the following sequence:

(SEQ ID NO: 40)
MEFTVTQKQRLQELGFEGVFPSDFEDVDERNRFFEELVGRLRDRNRKRFE
RLVGNKIPFWRKVSSDLRNRFYELGFVEVRTPEIISYSLLEKMEISDDLR
EQVYWLEEDNRCLRPMLAPNLYNELRHFNRISNQSKVRIFEIGTCFRREK
SSSEHLNEFTMLNAVEMGDIGDTEERLDRLIEEVFGEFTDYKKVGEESSL
YGKTVDVLVDGVEVASCIAGPHPLDSNWSIDQPWVGIGLGVERLAMLLDD
GSTAKAYGNSYIYQDGVRLDIK.

In an embodiment, the synthetase is a variant of TronΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 40.

FIG. 20 provides a sequence alignment. The aaRS may be at least 50%, 60%, 70%, 80%, 90%, 95%, 99%, or 100% identical to SEQ ID NO: 40, wherein the variation is found only in residues not marked with: i) “*”; ii) “*” or “:”, or iii) or “*”, “:” or “.”.

TronΔNTDPylRS, and its variants and derivatives, may be paired with a tRNA according to the following sequence:

(TrontRNA; SEQ ID NO: 52)
GGGGGGCTGGTCGGGGTGACCACGGAGGCTCTAAACCTCCCTTAGCCGGG
TTCAACTCCCGGGTCCCTCGCCA.

TrontRNA may be modified to include any anticodon. The tRNA may be at least 80%, 90%, 95%, 99%, or 100% identical to SEQ ID NO: 52.

Another aaRS disclosed herein is GemmΔNTDPylRS, which is an aaRS from Gemmatimonadetes bacterium (accession no. NNM03691.1). GemmΔNTDPylRS is an aaRS suitable for use in the prokaryotic cell of the present invention. The GemmΔNTDPylRS may have the following sequence:

(SEQ ID NO: 41)
MGITWSKTQKDRLRALRADGARLADSFEGRPQRDQAFQDLEGALAKARRK
ELEDLRAGHGRPGLCRLQTTLEGTLVGAGFVQVATPTIMSRGLLAKMGVT
KNHDLFEQVFWLDRDRCLRPMLAPHLYYVIKDLLRLWEKPLGIFEVGSCF
RKDSQGARHSNEFTMLNLCEFGLPEEDRGGRLREMAEVVTRAAGVHEYEL
EESASTVYGGTLDVVSVDGLELGSGAMGPHPLDHAWRITDTWVGIGFGLE
RLLMTVNRETSIGKMGRSLAYLDGIPLSI.

In an embodiment, the synthetase is a variant of GemmΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 41.

FIG. 20 provides a sequence alignment. The aaRS may be at least 50%, 60%, 70%, 80%, 90%, 95%, 99%, or 100% identical to SEQ ID NO: 41, wherein the variation is found only in residues not marked with: i) “*”; ii) “*” or “:”, or iii) or “*”, “:” or “.”.

GemmΔNTDPylRS, and derivatives, may paired with the tRNA GemmtRNA. The GemmtRNA may have the following sequence:

(SEQ ID NO: 46)
GGGGAGTGGATCGATACAAGATCGTGTGGGCTCTAAACCCACATAGCTCG
GTGTGACTCCGGGGCTCCCCACCA

GemmtRNA may be modified to include any anticodon. The tRNA may be at least 80%, 90%, 95%, 99%, or 100% identical to SEQ ID NO: 46.

Another aaRS disclosed herein is PGA8ΔNTDPylRS, which is an aaRS from Peptostreptococcaceae bacterium pGA-8 (accession no. SFE25427.1). PGA8ΔNTDPylRS is an aaRS suitable for use in the prokaryotic cell of the present invention. The PGA8ΔNTDPylRS may have the following sequence:

(SEQ ID NO: 42)
MQYSNTQRERLVQLNIDASLLEATFEDAEARDASFRSLEKELAKKAKTHL
RSLLSSDAVTGSQAVGNTLCSWLQQKGFTRVDTPTIISDKMLDKMSIDDK
HHLREQVFWIDRHKCLRPMLAPNLYIVMRELKKTLNTPVKIFEMGSCFRK
ESQGARHMNEFTMLNFVELATVKDGEQKEYLEKMAREAMAALKIDDYELI
IEESTVYGTTVDIEIDGIEVASGAYGPHELDSAWAVFDTWVGIGFGIERL
AMAMNGGKTIKRFGRSTNYLDGVPISL.

In an embodiment, the synthetase is a variant of PGA8ΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 42.

FIG. 20 provides a sequence alignment. The aaRS may be at least 50%, 60%, 70%, 80%, 90%, 95%, 99%, or 100% identical to SEQ ID NO: 42, wherein the variation is found only in residues not marked with: i) “*”; ii) “*” or “:”, or iii) or “*”, “:”, or “.”.

PGA8ΔNTDPylRS, and variants and derivatives, may paired with the tRNA PGA8tRNA. The PGA8tRNA may have the following sequence:

(SEQ ID NO: 54)
GGGGAGTGGATCGTATTGATCGTATGGACTCTAAATCCATAAAGCCGAGT
GAGACTCTCGGACTCCTCGCCA.

PGA8tRNA may be modified to include any anticodon. The tRNA may be at least 80%, 90%, 95%, 99%, or 100% identical to SEQ ID NO: 54.

Another aaRS disclosed herein is I2ΔNTDPylRS, which is an aaRS from Desulfosporosinus sp. 12 (accession no. WP_045576271.1). I2ΔNTDPylRS is an aaRS suitable for use in the prokaryotic cell of the present invention. The I2ΔNTDPylRS may have the following sequence:

(SEQ ID NO: 43)
MGIIWTPIQKQRLQELNASEAQREMCFESQQARDRAFQEQEHSLVVEGKR
RLMELRDIKRRPSLSVLEQQLVEALTQQGFVQVVTPTIISKTSLAKMSVS
DDHPLFSQVFWLDSKRCLRPMLAPNLYTLWKDLLRLWEKPIRIFEIGTCY
RKESKGSLHLNEFTMLNLTELGLPEDQRHQRLEELASLVMETVGIADYEM
ELTTSVVYGDTLDVVKGIELGSSAMGPHPLDDQWGIIDPWVGIGFGLERL
LMIKEGSQNVQSMGRSLTYLNGVRLNI.

In an embodiment, the synthetase is a variant of I2ΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 43.

FIG. 20 provides a sequence alignment. The aaRS may be at least 50%, 60%, 70%, 80%, 90%, 95%, 99%, or 100% identical to SEQ ID NO: 43, wherein the variation is found only in residues not marked with: i) “*”; ii) “*” or “:”, or iii) or “*”, “:” or “.”.

I2ΔNTDPylRS, and derivatives, may paired with any of the following tRNAs:

(I2CUAG; SEQ ID NO: 47)
GGGGGGTAGATCGGATTGATCGCGTGGACTCTAAATCCGCGCTAGACGGG
TGAAACTCCCGTACTCCTCGCCA
(I2b8; SEQ ID NO: 48)
GGGGCGTCGATCGGATTGATCGCGTGGACTCTAAATCCGCGCGCAACGGG
TGAAACTCCCGTACGCCTCGCCA
(I2b32; SEQ ID NO: 49)
GGGGTGTTGATCGGATTGATCGCGTGGACTCTAAATCCGCGGTAGACGGG
TGAAACTCCCGTACACCTCACCA
(I2b72; SEQ ID NO: 50)
GGGGTGTAGATCGGATTGATCGCGTGGACTCTAAATCCGCGAACAACGGG
TGAAACTCCCGTACACCTCGCCA
(I2H52; SEQ ID NO: 51)
GGGGCGTTGATCGGATTGATCGCGTGGACTCTAAATCCGCGGCCGACGGG
TGAAACTCCCGTACACCTCTCCA
(I2; SEQ ID NO: 55)
GGGGGGTAGATCGGATTGATCGCGTGGACTCTAAATCCGCGTAGACGGGT
GAAACTCCCGTACTCCTCGCCA

SEQ ID NOs: 47 to 51 and 55 may be modified to include any anticodon. The tRNA may be at least 80%, 90%, 95%, 99%, or 100% identical to any one of 47 to 51 and 55.

Another aaRS disclosed herein is D121ΔNTDPylRS, which is an aaRS from a Deltaproteobacteria (accession no. RLC04121.1). D121ΔNTDPylRS is an aaRS suitable for use in the prokaryotic cell of the present invention. The D121ΔNTDPylRS may have the following sequence:

(SEQ ID NO: 44)
MIPWTQTQTQRLNELNAPDSALKKSFDGEPAREKAYQALEKSLVTTQRRR
LAEFQTTHRRPELCRLENKLAEMLTQDGFAQVTTPIIMSRGLLKKMSIDS
KHPLNSQIFWLQEDKCLRPMLAPHLYYLLVDLLRIWKRPVRIFEIGPCFR
KESRGSKHASEFTMLNLVEMGTPENTRRERIQDVGSRVATAAGVKNYRFE
TVTSEIYGDTIDIVAGKDGLEIASAAMGPHPLDRPWKINESWIGIGFGLE
RLLMASHRSRNLARFGRSLAYLDGVRLNI.

In an embodiment, the synthetase is a variant of D121ΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 44.

FIG. 20 provides a sequence alignment. The aaRS may be at least 50%, 60%, 70%, 80%, 90%, 95%, 99%, or 100% identical to SEQ ID NO: 44, wherein the variation is found only in residues not marked with: i) “*”; ii) “*” or “:”, or iii) or “*”, “:” or “.”.

D121ΔNTDPylRS, and derivatives, may paired with the following tRNA:

(SEQ ID NO: 57)
GGGGGGTGGATCGTAATGAGATCGTGTGGACTCTAAATCCACATAGACGG
GTGCAACTCCCGTACTCCCCGCCA

SEQ ID NO: 57 may be modified to include any anticodon. The tRNA may be at least 80%, 90%, 95%, 99%, or 100% identical to SEQ ID NO: 57.

Another aaRS disclosed herein is D416ΔNTDPylRS, which is an aaRS from a Deltaproteobacteria (accession no. RTZ99416.1). D416ΔNTDPylRS is an aaRS suitable for use in the prokaryotic cell of the present invention. The D416ΔNTDPylRS may have the following sequence:

(SEQ ID NO: 45)
MNSSWTEVQRHRLKELNGAEKDLETAFGDDLQRNRAFQKLEKQLVYQERK
RLDRLLDTRFRPLRCELESLLIDALKCEGFTRVETPTIISQNDLERMSID
RSHPFNDQVYRVDSKHCLRPMLAPGLYRLMKDLARIRSGKPVRIFEIGPC
FRKETSGARHAGEFTMLNLVEMRIEKGSRRFRIETLAKRIMHAAGIDTYD
LVDEPSEVYNTTLDIVCGSDPLEVASCAMGPHPLDAAWGIIDTWVGLGFG
LERLLMARENSPGIGKWCKSVSYLDGIRLTL.

In an embodiment, the synthetase is a variant of D416ΔNTDPylRS further comprising mutations necessary to allow the recognition of, or to alter the specificity for, a non-canonical amino acid.

In an embodiment, the aaRS may be at least 50%, 60%, 70%, 80%, 90%, 95%, 99%, or 100% similar or identical to SEQ ID NO: 45.

FIG. 20 provides a sequence alignment. The aaRS may be at least 50%, 60%, 70%, 80%, 90%, 95%, 99%, or 100% identical to SEQ ID NO: 45, wherein the variation is found only in residues not marked with: i) “*”; ii) “*” or “:”, or iii) or “*”, “:”, or “.”.

D416ΔNTDPylRS, and derivatives, may paired with the following tRNA:

(SEQ ID NO: 58)
GGGGAGTGGATCGGATGAGATCGCATGGGCTCTAAACCCATGTAGCCGGG
TGCGACTCCCGGGCTTCCCTCCA

SEQ ID NO: 58 may be modified to include any anticodon. The tRNA may be at least 80%, 90%, 95%, 99%, or 100% identical to SEQ ID NO: 58.

In particular embodiments, any of the aaRSs disclosed herein may be modified to allow the recognition of, or to alter the specificity for, any of AllocK, AlkK, BocK, Bta, CbzK, CypK, 3-Nitro-Tyr, NMH, p-I-Phe, and p-Az-Phe, and o-Methyl-Tyrosine.

In an aspect of the invention, there is provided a host cell comprising any aaRS, or variant or derivative thereof, disclosed herein and any tRNA, or variant or derivative thereof, disclosed herein, wherein the aaRS and tRNA form an orthogonal aminoacyl-tRNA synthetase-tRNA pair. The host cell may be any suitable for heterologous protein production, in particular the production of polypeptides comprising one or more non-canonical amino acids. The host cell may be a prokaryotic or a eukaryotic cell. The host cell may be a bacterial cell, such as E. coli. The host cell may be a eukaryotic cell, such as a mammalian or insect cell line. The host cell may be a human cell. The host cell may be any bacterium of the invention as disclosed herein.

The aaRSs and tRNAs are also provided herein in the isolated forms.

The present inventors provide data herein demonstrating that the following orthogonal tRNA synthetase-tRNA pairs may be used in any combination of two or three pairs: i) MmPylRS/MmtRNA^{Pyl}_{YYY}; ii) 1R26PylRS/AlvtRNA^{ΔNPyl(8)}_{YYY}; iii) and AfTyrRS/Af-tRNA^{Tyr(A01)}_{YYY}.

Thus, in an aspect of the invention, there is provided a host cell comprising: any one, two, or three orthogonal tRNA synthetase-tRNA pairs selected from the group: i) MmPylRS/MmtRNA^{Pyl}_{YYY}; ii) 1R26PylRS/AlvtRNA^{ΔNPyl(8)}_{YYY}; iii) and AfTyrRS/Af-tRNA^{Tyr(A01)}_{YYY}. The aaRSs may be modified to allow the recognition of or alter the specificity for a non-canonical amino acid. The aaRSs may be any of the variants disclosed herein. The tRNAs may be any of the variants disclosed herein.

The present inventors provide data herein demonstrating that the following orthogonal tRNA synthetase-tRNA pairs may be used in any combination of two or three pairs: i) MmPylRS/MmtRNA^{Pyl}_{YYY}; ii) 1R26PylRS/AlvtRNA^{ΔNPyl(8)}_{YYY}; iii) and MjTyrRS/MjtRNA^{Tyr}_{YYY}.

Thus, in an aspect of the invention, there is provided a host cell comprising: any one, two, or three orthogonal tRNA synthetase-tRNA pairs selected from the group: i) MmPylRS/MmtRNA^{Pyl}_{YYY}; ii) 1R26PylRS/AlvtRNA^{ΔNPYl(8)}_{YYY}; iii) and MjTyrRS/MjtRNA^{Tyr}_{YYY}. The aaRSs may be modified to allow the recognition of or alter the specificity for a non-canonical amino acid. The aaRSs may be any of the variants disclosed herein. The tRNAs may be any of the variants disclosed herein.

In another aspect of the invention, there is provided a host cell comprising any one, two, or three orthogonal tRNA synthetase-tRNA pairs selected from the group: i) a class A ΔNPylRS/^{ΔNPyl}tRNA pair; ii) a class B ΔNPylRS/^{ΔNPyl}tRNA pair; and iii) an MmPylRS/Spe^{Pyl}tRNA pair. The classes of aaRS and tRNA are as described in Dunkelmann et al. (Engineered triply orthogonal pyrrolysyl-tRNA synthetase/tRNA pairs enable the genetic encoding of three distinct non-canonical amino acids, 2020).

The orthogonal tRNA synthetase-tRNA pairs may be expressed in a host cell. The host cell may be any suitable for heterologous protein production, in particular the production of polypeptides comprising one or more non-canonical amino acids. The host cell may be a prokaryotic or a eukaryotic cell. The host cell may be a bacterial cell, such as E. coli. The host cell may be a eukaryotic cell, such as a mammalian or insect cell line. The host cell may be a human cell. The host cell may be any bacterium of the invention as disclosed herein.

There is provided herein a bacterium that comprises a genome which comprises a polynucleotide sequence that is at least 80%, 85%, 90%, 95%, 98%, 99%, 99.5%, 99.8%, 99.9%, or 100% identical to SEQ ID NO: 8, and wherein the bacterium comprises a first orthogonal aminoacyl-tRNA synthetase-tRNA pair capable of decoding the sense codon TCG, a second orthogonal aminoacyl-tRNA synthetase-tRNA pair capable of decoding the sense codon TCA, and a third orthogonal aminoacyl-tRNA synthetase-tRNA pair capable of decoding the stop codon TAG. The calculation of the sequence identity of the genome should exclude the sequences required for expression of the orthogonal aminoacyl-tRNA synthetase-tRNA pairs (i.e. the bacterium may be of the strain Syn61Δ3(ev5), further modified to express the orthogonal aminoacyl-tRNA synthetase-tRNA pairs). In an embodiment, the orthogonal tRNA synthetase-tRNA pairs are: i) MmPylRS/MmtRNA^{Pyl}_{YYY}; ii) 1R26PylRS/AlvtRNA^{ΔNPyl(8)}_{YYY}; and iii) and AfTyrRS/Af-tRNA^{Tyr(A01)}_{CUA}, wherein the MmtRNA^{Pyl}_{YYY} may be MmtRNA^{Pyl}_{UGA}, the 1R26PylRS may be 1R26PylRS(CbzK), the AlvtRNA^{ΔNPyl(8)}_{YYY} may be AlvtRNA^{ΔNPyl(8)}_{CGA}, and the AfTyrRS may be AfTyrRS(p-I-Phe). In another embodiment, the orthogonal tRNA synthetase-tRNA pairs are: i) MmPylRS/MmtRNA^{Pyl}_{YYY}; ii) 1R26PylRS/AlvtRNA^{ΔNPyl(8)}_{YYY}; iii) and MjTyrRS/MjtRNA^{Tyr}_{CUA}, wherein the MmtRNA^{Pyl}_{YYY} may be MmtRNA^{Pyl}_{UGA}, the 1R26PylRS may be 1R26PylRS(CbzK), the AlvtRNA^{ΔNPyl(8)}_{YYY} may be AlvtRNA^{ΔNPyl(8)}_{CGA}, and the MjTyrRS may be MjTyrRS(3-Nitro-Tyr) or MjTyrRS(p-Az-Phe).

The substrate of the orthogonal tRNA synthetases that are expressed in prokaryotic cell of the invention may be any non-canonical amino acid. Hence, the prokaryotic cell of the invention may be used to generate polymers comprising a first non-canonical amino acid, a second non-canonical amino acid, and optionally a third non-canonical amino acid.

As used herein, the term “non-canonical amino acid” means any amino acid excluding L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan, and L-tyrosine.

The non-canonical amino acid may be an unnatural amino acid. As used herein, an “unnatural amino acid” is any amino acid that is not naturally encoded or found in the genetic code. Such amino acids may be non-proteinogenic amino acids. Thus, an unnatural amino acid may be any amino acid excluding L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan and L-tyrosine, L-pyrrolysine, and L-selenocysteine.

The non-canonical amino acids that are suitable for use with the present invention are not particularly limited. Suitable non-canonical amino acids will be well known to those of skill in the art, for example those disclosed in Neumann, H., 2012. FEBS letters, 586(15), pp. 2057-2064; and Liu, C. C. and Schultz, P. G., 2010. Annual review of biochemistry, 79, pp. 413-444 (herein incorporated by reference). In some embodiments the non-canonical amino acids are selected from one or more of: p-Acetylphenylalanine, m-Acetylphenylalanine, O-allyltyrosine, Phenylselenocysteine, selenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-Iodophenylalanine, p-Bromophenylalanine, p-Nitrophenylalanine, L-DOPA, 3-Aminotyrosine, 3-Iodotyrosine, p-Isopropylphenylalanine, 3-(2-Naphthyl)alanine, Biphenylalanine, Homoglutamine, D-tyrosine, p-Hydroxyphenyllactic acid, 2-Aminocaprylic acid, Bipyridylalanine, HQ-alanine, p-Benzoylphenylalanine, o-Nitrobenzylcysteine, o-Nitrobenzylserine, 4,5-Dimethoxy-2-nitrobenzylserine, o-Nitrobenzyllysine, o-Nitrobenzyltyrosine, 2-Nitrophenylalanine, Dansylalanine, p-Carboxymethylphenylalanine, 3-Nitrotyrosine, Sulfotyrosine, Acetyllysine, Methylhistidine, 2-Aminononanoic acid, 2-Aminodecanoic acid, Pyrrolysine, Cbz-lysine, Boc-lysine, Allyloxycarbonyllysine, N^{ε}-((tert-butoxy)carbonyl)-L-lysine (BocK), NE-(carbobenzyloxy)-L-lysine (CbzK), N^{ε}-allyloxycarbonyl-L-lysine (AllocK), (S)-2-Amino-3-(4-iodophenyl)propanoic acid (p-I-Phe), CypK, AlkK, 3-Nitro-Tyr, and p-Az-Phe. The first, second, and third non-canonical amino acid may be any combination of the aforementioned non-canonical amino acids.

In particular embodiments, the non-canonical amino acid may be any one of BocK, CbzK, AllocK, p-I-Phe, CypK, AlkK, 3-Nitro-Tyr, and p-Az-Phe (see Fig. S9). The first, second, and third non-canonical amino acid may be any combination of BocK, CbzK, AllocK, p-I-Phe, CypK, AlkK, 3-Nitro-Tyr, and p-Az-Phe.

### Uses of the Prokaryotic Cells of the Invention

The prokaryotic cells of the invention may be used to synthesise polymers or proteins that include a first, second, and/or third non-canonical amino acid. In particular embodiments, the prokaryotic cells of the invention may be used to synthesise polymers or proteins that include two or three different types of non-canonical amino acid. The non-canonical amino acids may be any as disclosed herein.

Thus, in an aspect of the invention, there is provided a method of synthesising a polymer, comprising:
i) providing a prokaryotic cell of the invention, wherein the prokaryotic cell comprises a first orthogonal aminoacyl-tRNA synthetase-tRNA pair; and contacting said prokaryotic cell with a nucleic acid sequence encoding a polymer, said nucleic acid sequence comprising the first type of sense codon;
ii) incubating the prokaryotic cell in the presence of a first non-canonical amino acid, wherein the first non-canonical amino acid is a substrate for the first orthogonal aminoacyl-tRNA synthetase; and
iii) incubating the prokaryotic cell to allow incorporation of the first non-canonical amino acid into the polymer via the first orthogonal aminoacyl-tRNA synthetase-tRNA pair.

In a particular embodiment, there is provided a method of synthesising a polymer, comprising:
i) providing a prokaryotic cell of the invention, wherein the prokaryotic cell comprises first and second orthogonal aminoacyl-tRNA synthetase-tRNA pairs; and contacting said prokaryotic cell with a nucleic acid sequence encoding a polymer, said nucleic acid sequence comprising the first type of sense codon and/or the second type of sense codon;
ii) incubating the prokaryotic cell in the presence of a first and a second non-canonical amino acid, wherein the first non-canonical amino acid is a substrate for the first orthogonal aminoacyl-tRNA synthetase and the second non-canonical amino acid is a substrate for the second orthogonal aminoacyl-tRNA synthetase; and
iii) incubating the prokaryotic cell to allow incorporation of the first non-canonical amino acid into the polymer via the first orthogonal aminoacyl-tRNA synthetase-tRNA pair and the incorporation of the second non-canonical amino acid into the polymer via the second first orthogonal aminoacyl-tRNA synthetase-tRNA pair.

In an embodiment, there is provided a method of synthesising a polymer, comprising: i) providing a prokaryotic cell of the invention, wherein the prokaryotic cell comprises first, second, and third orthogonal aminoacyl-tRNA synthetase-tRNA pairs; and contacting said prokaryotic cell with a nucleic acid sequence encoding a polymer, said nucleic acid sequence comprising a first type of sense codon, a second type of sense codon, and/or a first type of stop codon;
ii) incubating the prokaryotic cell in the presence of a first, a second, and a third non-canonical amino acid, wherein the first non-canonical amino acid is a substrate for the first orthogonal aminoacyl-tRNA synthetase, the second non-canonical amino acid is a substrate for the second orthogonal aminoacyl-tRNA synthetase, and the third non-canonical amino acid is a substrate for the third orthogonal aminoacyl-tRNA synthetase;
iii) incubating the prokaryotic cell to allow incorporation of the first non-canonical amino acid into the polymer via the first orthogonal aminoacyl-tRNA synthetase-tRNA pair, incorporation of the second non-canonical amino acid into the polymer via the second first orthogonal aminoacyl-tRNA synthetase-tRNA pair, and/or incorporation of the third non-canonical amino acid into the polymer via the third first orthogonal aminoacyl-tRNA synthetase-tRNA pair.

The contacting of the prokaryotic cell with the nucleic acid sequence may take any form that would provide the nucleic acid to the prokaryotic cell such that the nucleic acid sequence may be decoded to produce a polymer, such as a protein.

The prokaryotic cells for use in the present method may be any as described herein, wherein the prokaryotic cells comprise first and, optionally, second orthogonal aminoacyl-tRNA synthetase-tRNA pairs and, optionally, the third orthogonal aminoacyl-tRNA synthetase-tRNA pair. For instance, in an embodiment, the prokaryotic cell for use in the present method is a prokaryotic cell that does not express a first endogenous tRNA, a second endogenous tRNA, and a first endogenous release factor; and wherein a first type of sense codon and a second type of sense codon have been recoded within the genome of the prokaryotic cell such that the first endogenous tRNA and the second endogenous tRNA are dispensable, and a first type of stop codon has been recoded within the genome of the prokaryotic cell such that the first endogenous release factor is dispensable. The nature of the recoding may be as described for any prokaryotic cell of the invention disclosed herein. The first orthogonal tRNA is capable of decoding the first type of sense codon, the second orthogonal tRNA is capable of decoding the second type of sense codon, and the third orthogonal tRNA is capable of decoding the first type of stop codon. The prokaryotic cell may be E. coli.

In a particular embodiment, the prokaryotic cell for use in the present method is an E. coli cell that does not express tRNA^{Ser}_{UGA}, tRNA^{Ser}_{CGA}, or RF-1, occurrences of the sense codon TCA have been recoded such that tRNA^{Ser}_{UGA} is dispensable (e.g. occurrences of TCA in essential genes of the parent strain have been replaced with AGT), occurrences of the sense codon TCG have been recoded such that tRNA^{Ser}_{CGA} is dispensable (e.g. occurrences of TCG in essential genes of the parent strain have been replaced with AGC), and occurrences of the stop codon TAG have been recoded such that RF-1 is dispensable (e.g. occurrences of TAG in essential genes of the parent strain have been replaced with TAA). The bacterium has been modified to include first, second, and third orthogonal aminoacyl-tRNA synthetase-tRNA pairs capable of decoding the TCA, TCG, and TAG codons and capable of incorporating a first, second, and third non-canonical amino acid into such sites during polymer formation.

In particular embodiments, the prokaryotic cell for use in the present method is Syn61Δ3, Syn61Δ3(ev3), Syn61Δ3(ev4), or Syn61Δ3(ev5). As such, the prokaryotic cell may comprise a genome which is at least 80%, 85%, 90%, 95%, 98%, 99%, 99.5%, 99.8%, 99.9%, or 100% identical to any one of: SEQ ID NOs: 5 to 8. The calculation of the sequence identity percentage excludes any sequence that has been inserted to enable the expression of the orthogonal aminoacyl-tRNA synthetase-tRNA pairs.

The orthogonal tRNA synthetase-tRNA pairs may be any as described herein.

In particular embodiments the orthogonal tRNA synthetase-tRNA pairs are: MmPylRS/MmtRNA^{Pyl}_{YYY}; 1R26PylRS/AlvtRNA^{ΔNPyl(8)}_{YYY}; and AfTyrRS/Af-tRNA^{Tyr(A01)}_{YYY}. The MmtRNA^{Pyl}_{YYY} may be MmtRNA^{Pyl}_{UGA}. The 1R26PylRS may be 1R26PylRS(CbzK). The AlvtRNA^{ΔNPyl(8)}_{YYY} may be AlvtRNA^{ΔNPyl(8)}_{CGA}. The AfTyrRS may be AfTyrRS(p-I-Phe). The Af-tRNA^{Tyr(A01)}_{YYY} may be Af-tRNA^{Tyr(A01)}_{CUA}.

In other embodiments the orthogonal tRNA synthetase-tRNA pairs are: MmPylRS/MmtRNA^{Pyl}_{YYY}; 1R26PylRS/AlvtRNA^{ΔNPyl(8)}_{YYY}; and MjTyrRS/MjtRNA^{Tyr}_{YYY}. The MmtRNA^{Pyl}_{YYY} may be MmtRNA^{Pyl}_{UGA}. The 1R26PylRS may be 1R26PylRS(CbzK). The AlvtRNA^{ΔNPyl(8)}_{YYY} may be AlvtRNA^{ΔNPyl(8)}_{CGA}. The MjTyrRS may be MjTyrRS(3-Nitro-Tyr) or MjTyrRS(p-Az-Phe). The MjtRNA^{Tyr}_{YYY} may be MjtRNA^{Tyr}_{CUA}.

Fig. S5 presents data relating to the use of Syn61Δ3(ev5) to create proteins containing an ncAA in response to a sense codon or stop codon. As a control, MDS42 cells were also used create proteins containing a ncAA in response to a stop codon. As can be seen from this figure, the yield of the reporter protein containing an ncAA incorporated in response to an amber codon (as a ratio compared to wt) was vastly improved for Syn61Δ3(ev5) over the control. As such, the presently disclosed prokaryotic cells and methods of using said prokaryotic cells are surprising improvements over the prior art, even where producing the same product.

Further data relating to protein yields are provided in Table 1 (Data file S4).

Prokaryotic cells, e.g. E. coli, are not typically able to incorporate most eukaryotic post-translational modifications, such as ubiquitination, glycosylation and phosphorylation, nor are they typically capable of other eukaryotic maturation processes, and proteolytic protein maturation. In addition, correct disulphide bond formation and lipolysaccharide contaminations can be troublesome (see Ovaa, H., 2014. Frontiers in chemistry, 2, p. 15). However, therapeutic proteins, such as antibodies, enzymes and cytokines commonly carry post-translational modifications and disulphide bonds, and often require proteolytic maturation to attain their correctly folded state. Thus, the majority of therapeutic proteins are produced in eukaryotic and mammalian cell systems. However, expression in prokaryotic cells e.g. E. coli is in general cheaper, more susceptible to genetic modifications, and versatile with regard to mutant library development, and suitable for industrial scale fermentation (Ovaa, H., 2014. Frontiers in chemistry, 2, p. 15).

Thus, in some embodiments the synthesised polymers are therapeutic polypeptides, optionally wherein mammalian protein modifications have been introduced via one or more non-canonical amino acids. The synthesised polymer may be a protein, such as a therapeutic protein, comprising at least three distinct types of non-canonical amino acid.

Experimental data are provided herein that indicate that the prokaryotic cells of the invention may be used to generate polymers containing first, second and third non-canonical amino acids, each of which may differ from the others. Surprisingly, the inventors show that polymers may be produced that contain non-canonical amino acids that are directly adjacent to each other. In fact, the polymers may even contain only non-canonical amino acids. While it was known that ribosomes were able to tolerate one non-canonical amino acid at a time, it has never been shown that two non-canonical amino acids simultaneously within the same ribosome would allow productive peptide synthesis. As discussed in the Examples, for a linear polymer composed of two distinct monomers (A and B) there are four elementary polymerization steps (A+B->AB, B+A->BA, A+A->AA, B+B->BB) from which any sequence can be composed (FIG. 5A). For ribosome-mediated polymerization these four elementary steps correspond to each monomer acting as an A-site or P-site substrate to form a bond with another copy of the same monomer or with a distinct monomer (FIG. 5A). The experimental data presented in the Examples are the first, to the inventors' knowledge, to show that each of these four polymerization steps is possible.

Thus, in some embodiments the synthesised polymers, polypeptides, or proteins may include at least one non-canonical amino acid directly adjacent to another non-canonical amino acid. This polymer, polypeptide, or protein may include a chain of two, three, four, five, six, seven, eight, nine, ten, 15, 20, or more non-canonical amino acids directly adjacent to each other. The chain may comprise two or three distinct non-canonical amino acids.

In some embodiments, a polypeptide may be excisable from the synthesised polymer. For instance, the nucleic acid may encode cleavage or cleavable sites either side of the intended product. The excised product may be entirely made of non-canonical amino acids or may comprise one or more canonical amino acid. The excised product may be the chain of non-canonical amino acids described herein.

The synthesised polymer, including the linear or cyclic polymers, or protein may be purified after the method of synthesis. The purification may take any form known in the art in order to separate the polymer or protein from the cells and culture in which it was made.

The inventors demonstrate herein the use of prokaryotic cells of the invention for the production of cyclic polymers. The cyclic polymers may be generated via the synthesis of a polymer, as described herein, wherein the polymer is capable of self-cyclisation. The method may comprise the excision of a peptide from the polymer, wherein the peptide is capable of self-cyclisation. Said self-cyclisation may be a part of the excision process, but is not required to be so. The final product may be a macrocycle.

The macrocycle may include four, five, six, seven, eight, nine, ten, 15, 20, or more monomers. The macrocycle may include at least a first, second, and third non-canonical amino acid.

The prokaryotic cells of the invention can be used to generate products that are not obtainable by any other methods. As such, in an aspect of the invention, there is provided a polymer obtained from or obtainable by the methods of the invention. In an embodiment, the polymer comprises at least one first non-canonical amino acid, at least one second non-canonical amino acid, and optionally at least one third non-canonical amino acid, wherein the first, second, and third non-canonical amino acids are different from each other. The polymer may comprise least one non-canonical amino acid directly adjacent to another non-canonical amino acid. The polymer may include a chain of two, three, four, five, six, seven, eight, nine, ten, 15, 20, or more non-canonical amino acids directly adjacent to each other. The polymer may be a macrocycle or may be linear. Macrocycles of the present invention may comprise four, five, six, seven, eight, nine, ten or more monomers.

The prokaryotic cells of the invention may be suitable for the production of a target polymer or protein incorporating at least one non-canonical amino acid, wherein the rate of production is at least 5%, 10%, 15%, 25%, 35%, 50%, 60%, 70%, 80%, 90%, 100%, 110%, or 140% of the rate of production of the wild type control.

The prokaryotic cells of the invention may be suitable for the production of a target polymer or protein incorporating at least one non-canonical amino acid, wherein yield is at least 0.5, 1, 2, 5, 7.5, 10, 15, 20, or 25 mg/l.

### Methods of Making the Prokaryotic Cells of the Invention

Methods are provided herein for generating the prokaryotic cells of the invention. Aspects of said methods provide surprisingly effective processes for generating said prokaryotic cells and result in prokaryotic cells with surprisingly beneficial properties.

Thus, in an aspect of the invention, there is provided a method for producing a prokaryotic cell, wherein the method comprises:
(i) modifying a prokaryotic cell to express a first orthogonal aminoacyl-tRNA synthetase-tRNA pair suitable for decoding a first type of sense codon, wherein
   the prokaryotic cell comprises a genome wherein the first type of sense codon has been recoded such that a first endogenous tRNA is dispensable;
(ii) incubating the prokaryotic cell in the presence of a non-canonical amino acid which is a substrate for the first orthogonal aminoacyl-tRNA synthetase; and
(iii) modifying the endogenous gene encoding the first endogenous tRNA such that the first endogenous tRNA is not expressed.

Steps (ii) and (iii) may be performed before step (i).

In a particular embodiment, steps (i) and (ii) of the above method are performed before step (iii). The inventors have discovered that this particular order is surprisingly effective, and the prokaryotic cells have improved growth during the method. Without being bound to theory, the inventors hypothesise that the beneficial effects arise from a reduction in stalling on unannotated sense codons that may remain in the genome, hence this particular order of steps relieves this stalling and improves growth.

In a particular embodiment, there is provided a method for producing a prokaryotic cell, wherein the method comprises:
(i) modifying a prokaryotic cell to express a first orthogonal aminoacyl-tRNA synthetase-tRNA pair suitable for decoding a first type of sense codon, wherein
   the prokaryotic cell comprises a genome wherein the first type of sense codon has been recoded such that a first endogenous tRNA is dispensable;
(ii) incubating the prokaryotic cell in the presence of a non-canonical amino acid which is a substrate for the first orthogonal aminoacyl-tRNA synthetase; and
(iii) modifying the endogenous gene encoding the first endogenous tRNA such that the first endogenous tRNA is not expressed; and
(a) modifying the prokaryotic cell to express a second orthogonal aminoacyl-tRNA synthetase-tRNA pair suitable for decoding a second type of sense codon, wherein
   the prokaryotic cell comprises a genome wherein the second type of sense codon has been recoded such that a second endogenous tRNA is dispensable;
(b) incubating the prokaryotic cell in the presence of a non-canonical amino acid which is a substrate for the second orthogonal aminoacyl-tRNA synthetase; and
(c) modifying the endogenous gene encoding the second endogenous tRNA such that the second endogenous tRNA is not expressed.

The modifying of the endogenous gene encoding the first or second endogenous tRNA such that the first or second endogenous tRNA is not expressed, may be according to any technique that would prevent the production of a functional form of the endogenous tRNA within the prokaryotic cell. The functional form of an endogenous tRNA is a form that that would allow the decoding of the tRNAs cognate codon(s). For instance, the endogenous gene may be deleted or a portion of the gene may be deleted to prevent expression. Regulatory sequences may be deleted or altered to prevent expression. Alternatively, nonsense, frameshift, or missense mutations may prevent expression of the tRNA in a functional form.

Steps (ii) and (iii) may be performed before step (i), and steps (b) and (c) may be performed before step (a).

In a particular embodiment, steps (i) and (ii) of the above method are performed before step (iii). In addition, or alternatively, steps (a) and (b) may be performed before step (c). The inventors have discovered that this particular order is surprisingly effective, and the prokaryotic cells have improved growth during the method. Without being bound to theory, the inventors hypothesise that the beneficial effects arise from a reduction in stalling on unannotated sense codons that may remain in the genome, hence this particular order of steps relieves this stalling and improves growth.

The steps to introduce the second orthogonal tRNA synthetase-tRNA pair with the deletion of the dispensable endogenous tRNA (steps (a), (b), and (c)) may be performed before, at the same time, or after the steps to introduce the first orthogonal tRNA synthetase-tRNA pair and deleted the first endogenous tRNA ((steps (i), (ii), and (iii)).

In an embodiment, the method further comprises modifying the prokaryotic cell to express a third orthogonal aminoacyl-tRNA synthetase-tRNA pair suitable for decoding a first type of stop codon, wherein the first type of stop codon has been recoded within the genome of the prokaryotic cell such that a first endogenous release factor is dispensable.

This may be applied to any prokaryotic cell comprising a recoded genome with reduced occurrences of a first and second type of sense codon as discussed herein. For instance, the essential genes (as discussed herein) may not contain occurrences of the first or second type of sense codon. Alternatively, or in addition, the genome may comprise 5, 4, 3, 2, 1, or no occurrences of the first or second type of sense codon.

The method may further comprise modifying the prokaryotic cell to express a third orthogonal aminoacyl-tRNA synthetase-tRNA pair suitable for decoding a first type of stop codon, wherein a first type of stop codon has been recoded within the genome of the prokaryotic cell such that a first endogenous release factor is dispensable. This may be applied to any prokaryotic cell comprising a recoded genome with reduced occurrences of a first type of stop codon as discussed herein. For instance, the essential genes (as discussed herein) may not contain occurrences of the first type of stop codon. Alternatively, or in addition, the genome may comprise 5, 4, 3, 2, 1, or no occurrences of the first type of stop codon.

The methods for producing a prokaryotic cell may be, for instance, applied to an E. coli cell that does not express tRNA^{Ser}_{UGA}, tRNA^{Ser}_{CGA}, or RF-1, wherein occurrences of the sense codon TCA have been recoded such that tRNA^{Ser}_{UGA} is dispensable (e.g. occurrences of TCA in essential genes of the parent strain have been replaced with AGT), occurrences of the sense codon TCG have been recoded such that tRNA^{Ser}_{CGA} is dispensable (e.g. occurrences of TCG in essential genes of the parent strain have been replaced with AGC), and occurrences of the stop codon TAG have been recoded such that RF-1 is dispensable (e.g. occurrences of TAG in essential genes of the parent strain have been replaced with TAA).

In particular, the methods may be applied to Syn61, Syn61(ev1), or Syn61(ev2). As such, the methods may be applied to a bacterium that comprises a genome which is at least 80%, 85%, 90%, 95%, 98%, 99%, 99.5%, 99.8%, 99.9%, or 100% identical to any one of: GenBank accession number CP040347.1, SEQ ID NO: 3, or SEQ ID NO: 4.

The orthogonal tRNA synthetase-tRNA pairs inserted into the cells may be any suitable pairs, including any as described herein.

In particular embodiments the orthogonal tRNA synthetase-tRNA pairs are: MmPylRS/MmtRNA^{Pyl}_{YYY}; 1R26PylRS/AlvtRNA^{ΔNPyl(8)}_{YYY}; and AfTyrRS/Af-tRNA^{Tyr(A01)}_{CUA}. The MmtRNA^{Pyl}_{YYY} may be MmtRNA^{Pyl}_{UGA}. The 1R26PylRS may be 1R26PylRS(CbzK). The AlvtRNA^{ΔNPyl(8)}_{YYY} may be AlvtRNA^{ΔNPyl(8)}_{CGA}. The AfTyrRS may be AfTyrRS(p-I-Phe).

In other embodiments the orthogonal tRNA synthetase-tRNA pairs are: MmPylRS/MmtRNA^{Pyl}_{YYY}; 1R26PylRS/AlvtRNA^{ΔNPyl(8)}_{YYY}; and MjTyrRS/MjtRNA^{Try}_{CUA}. The MmtRNA^{Pyl}_{YYY} may be MmtRNA^{Pyl}_{UGA}. The 1R26PylRS may be 1R26PylRS(CbzK). The AlvtRNA^{ΔNPyl(8)}_{YYY} may be AlvtRNA^{ΔNPyl(8)}_{CGA}. The MjTyrRS may be MjTyrRS(3-Nitro-Tyr) or MjTyrRS(p-Az-Phe).

The inventors have discovered that, unless specific steps are taken, the methods of producing prokaryotic cell may result in growth defects. To overcome these growth defects the inventors make use of methods of mutagenesis and selection. In a particular embodiment, such method steps are performed before the method steps for producing a prokaryotic cell disclosed herein. It is surprising that such methods may be applied in the present context. The inventors initially assumed that the growth defect may be due to a limiting amount of serV, but further experiments revealed this not to be the case (Fredens et al., 2019). As such, it was unclear whether the growth defect was surmountable. In addition, the inventors assumed that random mutagenesis and selection might reverse some of the recoding of the genome and could also potentially have resulted in read-through of the blank codons. However, as demonstrated by the experiments disclosed herein, the inventors were able to reverse the growth defects without a loss of desired functionality.

Thus, in an embodiment, prior to the steps of the method for producing a prokaryotic cell, the method comprises: inducing mutagenesis in a cell culture comprising any prokaryotic cell that comprises a recoded genome disclosed herein; maintaining the cell culture under exponential growth conditions; selecting a prokaryotic cell from said cell culture with an increased growth rate compared to the initial culture; wherein the steps of the method for producing a prokaryotic cell are applied to the selected prokaryotic cell.

The mutagenesis, mutation, and selection steps may be part of a parallel mutagenesis and dynamic parallel selection process, as disclosed herein. The parallel mutagenesis and dynamic parallel selection process may be as disclosed in Schmied et al. (W. H. Schmied et al., Controlling orthogonal ribosome subunit interactions enables evolution of new function. Nature 564, 444-448 (2018); herein incorporated by reference).

In an embodiment, the induction of mutagenesis comprises the use of a mutagenesis plasmid.

In particular embodiments, the induction of mutagenesis comprises the use of the mutagenesis plasmid “MP6”. MP6 is described in Badran et al. (A. H. Badran, D. R. Liu, Development of potent in vivo mutagenesis plasmids with broad mutational spectra. Nature Communications 6, 8425 (2015); herein incorporated by reference). MP6 may be according to SEQ ID NO: 59.

In some embodiments, the mutagenesis may be carried out for 5, 10, 15, 17, 20, 30, 45, 60, 70, 80, 100, 150, 200, or more generations. In particular embodiments, the mutagenesis may be carried out for approximately 1-200, 5-100, 10-25, 15-20, or 17 generations.

In some embodiments, after mutagenesis, the mutator cultures may be maintained at continuous growth. For instance, the mutator cultures may be transferred to multiple vessels (such as the wells of a 96 well plate). The OD₆₀₀ of the culture may be measured after transfer and then periodically again every 2-9 h, depending on the growth rate. When the OD₆₀₀ reaches a threshold of 0.5 for at least 3 mutator cultures, all cultures may be diluted (for instance, 1/400) into fresh vessels. If no cultures reach the threshold, the three highest OD₆₀₀ values may be used to estimate the next OD₆₀₀ measurement time point, i.e. the time required to reach the threshold. If fewer than 3 cultures reach the threshold after three consecutive OD₆₀₀ measurements, the cultures may be incubated for another 12 h before a dilution step regardless of the measured cell density. Overall, this process flexibly adjusts the selective pressure in an effort to keep the fastest growing cultures in exponential growth.

In some embodiments, the cell culture may be maintained under exponential growth conditions for 5, 10, 15, 20, 30, 40, 50, 52, 60, 70, 80, 100, 200, or more generations. In particular embodiments, the cell culture may be maintained under exponential growth conditions for approximately 20-80, 30-70, 40-60, or 50 generations. In an exemplified embodiment, the number of generations is 52.

Multiple rounds of mutagenesis, maintenance, and selection may be applied to the cell cultures. For instance, one, two, three, four, five, ten or more rounds may be applied. In particular embodiments, two rounds are applied. In an embodiment, each round includes the induction of mutagenesis for approximately 10-25 (e.g. 17) generations, and maintenance under exponential growth conditions for approximately 20-80 (e.g. 50) generations.

The mutagenesis, mutation, and selection steps may be applied to Syn61, Syn61(ev1), or Syn61(ev2). As such, the methods may be applied to a cell that comprises a genome which is at least 80%, 85%, 90%, 95%, 98%, 99%, 99.5%, 99.8%, 99.9%, or 100% identical to any one of: the sequence provided in GenBank accession number CP040347.1, SEQ ID NO: 3, or SEQ ID NO: 4.

The inventors have further discovered that, unless specific steps are taken, the removal of a first endogenous tRNA, a second endogenous tRNA, and/or a first endogenous release factor may result in growth defects. To overcome these growth defects the inventors make use of methods of mutagenesis and selection. In a particular embodiment, such method steps are performed after the method steps for producing a prokaryotic cell disclosed herein. As for the steps prior to the removal of the endogenous genes, it was assumed that random mutagenesis and selection might reverse some of the desired technical features of the prokaryotic cells, for instance leading to read-through of the blank codons. However, again, the inventors surprisingly found that the growth defects could be remedied without a loss of desired functionality.

Thus, in an embodiment, after the steps of the method for producing a prokaryotic cell, the method further comprises: obtaining a cell culture from the prokaryotic cell; inducing mutagenesis in the cell culture; maintaining the cell culture under exponential growth conditions; and selecting a prokaryotic cell from said cell culture with an increased growth rate compared to the initial culture.

The mutagenesis, mutation, and selection steps may be part of a parallel mutagenesis and dynamic parallel selection process, as disclosed herein. The parallel mutagenesis and dynamic parallel selection process may be as disclosed in Schmied et al. (W. H. Schmied et al., Controlling orthogonal ribosome subunit interactions enables evolution of new function. Nature 564, 444-448 (2018); herein incorporated by reference).

The inventors have discovered that the mutagenesis plasmid MP6 is not functional in cells lacking endogenous tRNAs. To circumvent this problem the inventors have designed a new plasmid, referred to as “recoded MP6”. This plasmid may be according to SEQ ID NO: 67.

In other embodiments, the mutagenesis plasmid may be a known plasmid, wherein the plasmid has been recoded to remove the first type of sense codon, the second type of sense code, and/or the first type of stop codon, and to replace said codons with synonymous codons. For instance, the mutagenesis plasmid might contain only five, four, three, two, one, or no occurrences of the first type of sense codon, second type of sense codon, or first type of stop codon. In a particular embodiment, the mutagenesis plasmid does not contain any occurrences of the first or second type of sense codon nor any occurrences of the first type of stop codon.

As such, there is provided herein the use of a recoded MP6 or a recoded mutagenesis plasmid for inducing mutagenesis in a recoded prokaryotic cell. Such use may form part of the methods for producing a prokaryotic cell of the invention, as disclosed herein.

In some embodiments, the mutagenesis may be carried out for 5, 10, 15, 17, 20, 30, 45, 60, 70, 80, 100, 150, 200, or more generations. In particular embodiments, the mutagenesis is carried out for at least 20, 25, 30, 35, or 40 generations. In particular embodiments, the mutagenesis may be carried out for approximately 1-200, 5-100, 10-90, or 25-75 generations. In particular examples, the mutagenesis may be carried out for at least or about 45, 60, 70 generations.

In some embodiments, after mutagenesis, the mutator cultures may be maintained at continuous growth. This process may be the same as discussed in relation to steps to be performed prior to the removal of the endogenous tRNAs and release factor.

In some embodiments, the cell culture may be maintained under exponential growth conditions for 5, 10, 15, 20, 30, 40, 50, 52, 60, 70, 80, 100, 200 or more generations. In particular embodiments, the cell culture may be maintained under exponential growth conditions for approximately 20-80, 30-70, 40-60, or 50 generations. In an exemplified embodiment, the number of generations is 52.

Multiple rounds of mutagenesis, maintenance, and selection may be applied to the cell cultures. For instance, one, two, three, four, five, ten or more rounds may be applied. In particular embodiments, three rounds are applied. In an embodiment, each round includes the induction of mutagenesis for at least 20 generations, and maintenance under exponential growth conditions for approximately 20-80 (e.g. 50) generations. In a particular embodiment, the first round may include approximately 70 generations, the second round may include 45 generations, and the third round may include 60 generations of mutagenesis.

At any stage during the methods for producing a prokaryotic cell of invention, the method may comprise a phenotyping assay. The phenotyping assay comprises the analysis of whether the first type of sense codon, second type of sense codon, and first type of stop codon remain recoded. The assay may comprise transforming the prokaryotic cell with a reporter plasmid comprising a recoded codon linked to a reporter, such as GFP (suitable reporters are discussed in the Examples). An orthogonal aaRS/tRNA pair, capable of decoding the recoded codon to incorporate a ncAA, is also inserted. The assay may then comprise the incubation of the cells in the presence or absence of the ncAA substrate to determine if there is reporter signal that is dependent on the ncAA. Further details are provided in the Examples.

As noted above, the inventors have surprisingly discovered that the methods of mutagenesis and selection may be applied to prokaryotic cells with recoded genomes without affecting the recoding.

Thus, in an aspect of the invention, there is provided a method for improving the growth rate of a prokaryotic cell wherein the genome has been recoded, wherein the method comprises
inducing mutagenesis in a cell culture comprising the prokaryotic cell that comprises the recoded genome,
maintaining the cell culture under exponential growth conditions,
selecting a prokaryotic cell, from said cell culture, with an increased growth rate compared to the initial culture.

The prokaryotic cell may comprise a genome wherein a first type of sense codon has been recoded. The recoding of a first type of sense codon may be any sense codon, including any recoded type of sense codon as disclosed herein. The prokaryotic cell may comprise a genome wherein a first and a second type of sense codon has been recoded. The recoding of a first and second types of sense codon may be any two sense codons, including any recoded type of sense codon as disclosed herein.

The methods of inducing mutagenesis, maintaining the cells, and selecting the cell may be any as disclosed herein.

### Methods for Recoding a Genome

The following methods enable the skilled person to recode genomes to replace sense or stop codons with synonymous codons. Further information is available in: i) K. Wang et al., Defining synonymous codon compression schemes by genome recoding. Nature 539, 59-64 (2016); ii) J. Fredens et al., Total synthesis of Escherichia coli with a recoded genome. Nature 569, 514-518 (2019); iii) WO2018/020248, and iv) WO2020/229592 (each of which is incorporated herein by reference).

In an example, preferably one or more rounds of recombination-mediated genetic engineering are used to edit 10-1000 kb, 50-1000 kb, 100-1000 kb, or 100-500 kb of a parent genome to provide two or more different partially synthetic genomes. Thus, in preferred examples each round of recombination-mediated genetic engineering inserts or replaces 10 kb or more, 50 kb or more, 100 kb or more, or about 100 kb of DNA in the parent genome.

As used herein, the term “recombination-mediated genetic engineering” (also known as “recombineering”) is a method for genetic engineering (i.e. editing genomes) based on homologous recombination systems. Typically recombineering is based on homologous recombination in Escherichia coli mediated by bacteriophage proteins, either RecE/RecT from Rac prophage or Redαβδ from bacteriophage lambda. Any suitable method of recombination-mediated genetic engineering may be used. Methods for recombination-mediated genetic engineering will be well known to those of skill in the art.

In “classical recombination” (exemplified by lambda red mediated recombination in E. coli), short regions of synthetic DNA may be inserted into the genome or used to replace genomic DNA in a two-step process: i) transformation of cells with linear double stranded DNA (dsDNA) carrying a stretch of synthetic DNA, coupled with a positive selection marker, and flanked by a homology region (HR) to the target region of the genome on each end, and ii) recombination mediated by the homologous regions, followed by selection for genomic integration by virtue of the positive selection marker. This approach can be used to insert or replace 2-3 kb of genomic DNA. Thus, if classical recombination is used, many rounds of recombination-mediated genetic engineering would be required to edit 100-500 kb of the parent genome.

Thus, in preferred examples the one or more rounds of recombination-mediated genetic engineering comprise one or more rounds of replicon excision for enhanced genome engineering through programmed recombination (REXER).

REXER is described in WO 2018/020248. Each round of REXER may be used to insert or replace about 50 kb to 250 kb, or about 100 kb of DNA in the parent genome.

Thus, the one or more rounds of recombination-mediated genetic engineering may comprise:
i) providing a host cell (e.g. E. coli), wherein the host cell comprises an episomal replicon (e.g. a plasmid or a bacterial artificial chromosome) and a target nucleic acid (e.g. the genome), wherein the episomal replicon comprises a donor nucleic acid sequence (i.e. a synthetic region), wherein the donor nucleic acid sequence comprises in order: 5′-homologous recombination sequence 1-sequence of interest-homologous recombination sequence 2-3′, wherein the sequence of interest comprises a positive selectable marker, and wherein the target nucleic acid comprises in order: 5′-homologous recombination sequence 1-negative selectable marker-homologous recombination sequence 2-3′;
ii) providing helper protein(s) capable of supporting nucleic acid recombination in said host cell (e.g. lambda Red proteins);
iii) providing helper protein(s) and/or RNAs capable of supporting nucleic acid excision in said host cell (e.g. CRISPR/Cas9 proteins/RNAs);
iv) inducing excision of said donor nucleic acid sequence;
v) incubating to allow recombination between the excised donor nucleic acid and said target nucleic acid; and
vi) selecting for recombinants having incorporated said donor nucleic acid into said target nucleic acid.

Suitably selecting for recombinants having incorporated said donor nucleic acid into said target nucleic acid comprises selection for gain of the positive selectable marker of the donor nucleic acid and loss of the negative selectable marker of the target nucleic acid. Suitably selection for gain of the positive selectable marker of the donor nucleic acid and loss of the negative selectable marker of the target nucleic acid is carried out simultaneously. Suitably said sequence of interest comprises both a positive selectable marker and a negative selectable marker. Suitably the negative selectable marker is selected from the group consisting of sacB (sucrose sensitivity), rpsL (S12 ribosomal protein-streptomycin sensitivity), or phe^{ST2511A_A294G} (4-chlorophenylalanine sensitivity). Suitably the positive selectable marker is selected from the group consisting of Cm^{R}(chloramphenicol resistance), Kan^{R} (kanamycin resistance), Hyg^{R} (hygromycin resistance), Gentamycin^{R} (gentamycin resistance), or tetracycline^{R} (tetracycline resistance). Suitably the step of selecting for recombinants comprises sequential selection for said positive and negative markers, or sequential selection for said negative and positive markers. Suitably the step of selecting for recombinants comprises simultaneous selection for said positive and negative markers.

Suitably said method as described above further comprises the step of inducing at least one double stranded break in the target nucleic acid sequence, wherein said double stranded break is between said homologous recombination sequence 1 and said homologous recombination sequence 2. Suitably at least two double stranded breaks are induced in the target nucleic acid sequence, wherein each said double stranded break is between said homologous recombination sequence 1 and said homologous recombination sequence 2.

Suitably said excised donor nucleic acid begins with said homologous recombination sequence 1 and ends with said homologous recombination sequence 2.

Suitably said episomal replicon comprises a negative selectable marker independent of the donor nucleic acid sequence. Suitably said method comprises the further step of selecting for loss of the episomal replicon by selecting for loss of said negative selectable marker independent of the donor nucleic acid sequence. Suitably said episomal replicon comprises in order: excision cut site 1-donor nucleic acid sequence-excision cut site 2. Suitably said target nucleic acid possesses its own origin of replication capable of functioning within said host cell. Suitably said episomal replicon is a plasmid nucleic acid. Suitably said episomal replicon is a bacterial artificial chromosome (BAC). Suitably said target nucleic acid is the host cell genome.

The episomal replicon (e.g. BAC) may be assembled by homologous recombination, for example in S. cerevisiae, as described in Kouprina, N., et al., 2004. Methods Mol Biol 255, 69-89. The assembly may combine: 7-14 stretches of synthetic DNA, each 6-13 kb in length; a selection construct (comprising a negative selection marker and/or a positive selection marker); and a BAC shuttle vector backbone. The stretches of synthetic DNA may collectively correspond to the donor nucleic acid sequence (i.e. the synthetic region) in the episomal replicon, wherein each stretch comprises 80-200 bp of overlapping DNA sequence with each other, and wherein the overlap regions are free of any recoding targets. The stretches may be supplied in pSC101 or pST vectors flanked by suitable restriction sites (e.g. BsaI, AvrII, SpeI, or XbaI). Thus, during assembly the synthetic DNA stretches may be excised by digestion with the corresponding restriction enzymes. Assembly of the episomal replicon may be verified by sequencing.

Suitably the two homology regions may be 30-100 bp, or 40-50 bp, or about 50 bp in length.

CRISPR/Cas9 machinery may be used to for excision. In some examples the CRISPR/Cas9 machinery comprises Cas9, tracrRNA and two spacer RNAs, wherein the spacer RNAs target the two homology regions for excision. In preferred embodiments, the spacer RNAs are linear double stranded spacers. In other embodiments, the CRISPR/Cas9 machinery comprises Cas9 and two sgRNAs, wherein the sgRNAs target the two homology regions for excision.

Lambda red recombination machinery may be used for recombination. The lambda red recombination machinery may comprise lambda alpha/beta/gamma.

The method may comprise performing one or more rounds of REXER, i.e. the steps as described above with a first donor nucleic acid sequence, choosing further donor sequence(s) contiguous with said first donor nucleic acid sequence, and repeating said steps with said further donor nucleic acid sequence(s) until the partially synthetic genome has been assembled. This is known as genome stepwise interchange synthesis (GENESIS), described in Wang, K. et al., 2016. Nature 539, 59-64 and is shown schematically in FIG. 4 of WO2020/229592.

In preferred examples the donor sequence(s) correspond to regions of the synthetic genome.

Thus, the donor sequence(s) (i.e. synthetic region) may comprise 20 or fewer occurrences of one or more sense codons; and/or the donor sequence(s) may comprise 10 or more, 20 or more, or 100 or more genes with no occurrences of one or more sense codons.

The donor sequence(s) (i.e. synthetic region) may be identical to sequences (i.e. non-synthetic regions) of the parent genome except that they have 50 or fewer, 20 or fewer, 10 or fewer, 5 or fewer, or 0 occurrences of each of one or more sense codons; and/or comprise less than 10%, 5%, 2%, 1%, 0.5%, 0.1% of the occurrences of each of one or more sense codons, relative to the corresponding region in the parent genome; and/or comprise 10 or more, 20 or more, or 100 or more genes with no occurrences of one or more sense codons.

The donor sequence(s) (i.e. synthetic region) may also be refactored relative to the sequences (i.e. non-synthetic regions) of the parent genome. For 3′,3′ overlaps (i.e. pairs of genes in opposite orientations) a synthetic insert may be inserted between the genes. For 3′,3′ overlaps the synthetic insert may comprise the overlapping region. For 5′, 3′ overlaps (i.e. pairs of genes in the same orientation) a synthetic insert may be inserted between the genes. For 5′, 3′ overlaps the synthetic insert may comprise: (i) a stop codon; (ii) about 20-200 bp, or 20-100 bp, or 20-50 bp, from upstream of the overlapping region; and (iii) the overlapping region. Preferably, the synthetic insert comprises: (i) a stop codon; (ii) about 20 bp from upstream of the overlapping region; and (iii) the overlapping region. In preferred embodiments the stop codon is in frame with the original start site for the downstream gene. Preferably the stop codon is TAA.

Preferably the donor sequence(s) (i.e. synthetic region) are collectively 50-10000 kb, 100-5000 kb, 100-2000 kb, 100-1000 kb, or 100-500 kb in size. Preferably each donor sequence is 50-300 kb, 100-200 kb, or about 100 kb in size.

Accordingly, the donor sequences may each be about 100 kb in size and identical to corresponding sequences of the parent genome, except they comprise no occurrences of one or more sense codons and all pairs of genes which share an overlapping region comprising the one or more sense codons in the parent genome are refactored, wherein the pairs of genes are those in which sense codon replacements would change the encoded protein sequence of both or either of the pair of genes.

In preferred examples the viability of the genome is tested after each round of recombination-mediated genetic engineering. In some examples the sequence of the genome is verified after each round of recombination-mediated genetic engineering.

In some embodiments the genome is a reduced synthetic genome or a minimal synthetic genome. A “reduced genome” is one in which the size of the parent genome has been reduced by removing non-essential genes and/or non-coding regions. A “minimal genome” is a genome which has been reduced to its minimal size whilst remaining viable e.g. by deletion of all non-essential regions of the genome.

### Refactoring

Genomes contain numerous overlapping open reading frames (ORFs), which can be classified as 3′, 3′ (between ORFs in opposite orientations) or 5′, 3′ (between ORFs in the same orientation). The sense codons to be replaced may be found within both classes of overlap in the parent genome.

If the replacement of the sense codons of each ORF within an overlap can be achieved without changing the encoded protein sequence of either ORF (i.e. by introducing synonymous codon(s)) then it may not be necessary to edit (e.g. refactor) the parent genome. However, when the encoded protein sequence is changed by the replacement of the sense codons, (i.e. one or more synonymous sense codons are not introduced into one or both of the ORFs), then it may be necessary to edit (e.g. refactor) the parent genome.

Thus, in some examples one or more pairs of genes which share an overlapping region comprising the sense codons in the parent genome are refactored. “Refactored” means that the genes are reorganised to prevent changes to the encoded protein sequences. Preferably, the pairs of genes are those in which sense codon replacements (e.g. defined synonymous codon replacements) would change the encoded protein sequence of both or either of the pair of genes. Most preferably, all pairs of genes which share an overlapping region comprising the sense codons in the parent genome are refactored, wherein the pairs of genes are those in which sense codon replacements (e.g. defined synonymous codon replacements) would change the encoded protein sequence of both or either of the pair of genes.

For 3′,3′ overlaps (i.e. pairs of genes in opposite orientations) a synthetic insert may be inserted between the genes. For 3′,3′ overlaps the synthetic insert may comprise the overlapping region.

For 5′, 3′ overlaps (i.e. pairs of genes in the same orientation, comprising an upstream gene and a downstream gene) a synthetic insert may be inserted between the genes. For 5′,3′ overlaps the synthetic insert may comprise: (i) a stop codon; (ii) about 20-200 bp, or 20-100 bp, or 20-50 bp, from upstream of the overlapping region; and (iii) the overlapping region.

Preferably, the synthetic insert comprises: (i) a stop codon; (ii) about 20 bp from upstream of the overlapping region; and (iii) the overlapping region. This preserves the sequence of the RBS for the downstream ORF and the distance between this RBS and its start codon. In preferred embodiments the stop codon is in frame with the original start site for the downstream gene. Preferably the stop codon is TAA.

### General

In all instances, where a “prokaryotic cell” described, the invention also extends to a cell culture containing a plurality of the prokaryotic cells. Similarly, where a “bacterium” is described, the invention also extends to a bacterial culture containing a plurality of the bacteria.

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences. In order to calculate the percentage identity between two nucleic sequences, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle(EMBOSS) or Stretcher(EMBOSS), the Smith-Waterman algorithm (e.g. as applied by Water(EMBOSS)), or the LALIGN application (e.g. as applied by Matcher(EMBOSS); and (ii) the parameters used by the alignment method, for example, local versus global alignment, the matrix used, and the parameters applied to gaps.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two nucleic sequences may be calculated using the service Needle(EMBOSS) set to the default parameters, e.g. matrix (BLOSUM62), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (BLOSUM62), gap open (1), gap extend (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

It is widely hypothesised that removing cellular tRNAs—making their cognate codons unreadable—might create a genetic firewall to viral infection and enable sense codon reassignment. However, it has been impossible to test these hypotheses because no such bacteria have been created. Here—following synonymous codon compression and laboratory evolution in Escherichia coli—the inventors delete the tRNAs and release factor-1, which normally decode two sense codons and a stop codon; the resulting cells cannot read the canonical genetic code and are completely resistant to a cocktail of viruses. The inventors reassign these codons to enable the efficient synthesis of proteins containing three distinct non-canonical amino acids. Importantly, the inventors demonstrate the facile reprogramming of the cells for the encoded translation of diverse non-canonical heteropolymers and macrocycles.

### Example 1—Creating Syn61Δ3

The inventors predicted that replacing the annotated TCA, TCG and TAG codons in the genome of a bacteria cell would enable deletion of serT and serU (encoding tRNA^{Ser}_{UGA} and tRNA^{Ser}_{CGA}) and prfA (encoding RF-1), which decode these codons, in a single strain (FIG. 1A). The inventors have previously showed that serT, serU and prfA could be deleted in separate strains derived from Syn61 (18); however, this does not capture the potential epistasis between these genes. As such, the experiments disclosed herein asked whether serT, serU and prfA could be deleted in a single strain derived from Syn61.

Syn61 grows 1.6-fold slower than the strain from which it was derived (18). To increase the growth rate of the strain prior to serT, serU and prfA deletion, a previously described random parallel mutagenesis and automated dynamic parallel selection strategy was applied (19); this approach uses feedback control to dynamically dilute mutated cultures on the basis of growth rate, and thereby selects fast growing strains from within mutated populations (Fig. S1A). Through two consecutive rounds of mutagenesis and selection a strain was created, Syn61(ev2), which grew 1.3-fold faster (FIG. 1B, Figs. S1B, C, D, and E, and Data files S1 and S2).

Next, serU, serT and prfA were removed from Syn61(ev2) to create Syn61Δ3 (FIG. 1A, Fig. S1C, and Data files S1 and S2). This demonstrated that removing the target codons in Syn61 was sufficient to enable the deletion of all decoders of the target codons in the same strain. However, Syn61Δ3 grew 1.7-fold slower than Syn61(ev2) (FIG. 1B). This growth decrease may result from the presence of target codons in the genome of Syn61 that were not annotated and targeted (20, 21), and may also result from the other non-canonical roles that tRNAs may play (22, 23).

Three sequential rounds of random parallel mutagenesis and automated dynamic parallel selection were performed in order to evolve Syn61Δ3 to Syn61Δ3(ev5), which grew 1.6-fold faster than Syn61Δ3 (FIGS. 1A and B, Figs. S1B, C, F, G, and H, and Data file S1). When grown in LB media in shake flasks the doubling time of Syn61Δ3(ev5) was 38.72+/−1.02 min (Fig. S1I). Syn61Δ3(ev5) contains 482 additional mutations—420 substitutions and 62 indels—of which 72 are in intergenic regions, with respect to Syn61 (Data files S1 and S3, Fig. S2). No target codons were reverted, further demonstrating the stability of our recoding scheme. 16 sense codons in non-essential genes were converted to target codons (5×TCG, 3×TCA, 8×TAG); these frequencies are comparable to those observed for other codons (Data file S1). Subsequent experiments used Syn61Δ3 or (once available) its evolved derivatives to investigate the new properties of these strains.

### Materials and Methods for Example 1

### Gene Recoding

For genes and plasmids used in this work it was necessary to compress the genetic code. The genetic code of protein CDSs was compressed using the recoding rules implemented in the design of Syn61 (TCG to AGC, TCA to AGT, TAG to TAA) using a custom Python script (18)—see Data file S2 for sequence details.

### Construction of Recoded Helper and MP6 Plasmid

All cloning procedures from this section were performed in E. coli DH10b or MDS42, both strains containing an rpsLK43R mutation for resistance to streptomycin. The helper plasmid used throughout this study for all lambda-red-mediated recombination experiments is a recoded version of the ‘helper’ plasmid pKW20_CDFtet_pAraRedCas9_tracrRNA described in ref (18), which is referred to herein as ‘recoded helper’; see Data file S2 for sequence details.

To construct the recoded helper plasmid the sequence was first divided into five sections with 20-25 bp overlaps: i) section 1 encoding araC, pBAD promoter and lambda-red gamma, ii) section 2 encoding lambda-red beta and alpha followed by a unique BsaI site, iii) section 3 encoding Cas9 flanked by BsaI sites, iv) section 4 encoding an apraR selection marker, and v) section 5 encoding the pCDF origin, a tracrRNA cassette and an ampicillin promoter.

Sections 1-3 were synthesized by Genewiz, section 4 was synthesized as a gBlock (IDT) and section 5 was amplified by PCR from the original helper plasmid (18).

To assemble the recoded helper plasmid, firstly sections 4 and 5 were combined by overlap-extension PCR. The product was then assembled together with sections 1 and 2 into an intermediate plasmid by HiFi Assembly (NEB). The intermediate plasmid was transformed into E. coli MDS42 and its sequence verified by next generation sequencing (NGS). Finally, the intermediate plasmid was digested with BsaI, and assembled together with section 3 by HiFi Assembly to generate the recoded helper plasmid. The plasmid sequence was verified by NGS.

The inventors designed a recoded version of MP6 mutagenesis plasmid (31) for Syn61 strain evolution. The recoded sequence was divided into four overlapping sections, so as to minimise expression of the mutagenic components during cloning: i) section 1 encoding araC and the pBAD promoter, ii) section 2 encoding most of dnaQ926 followed by an AvrII site, PmCDA1, and cat for chloramphenicol resistance, iii) section 3 encoding the N-terminus of dnaQ926 followed by dam, seqA, emrR, and ugi and iv) section 4 containing the pCDF origin. Sections 1-3 were synthesised by Genewiz and Twist Bioscience, while section 4 was amplified by PCR from the original MP6 plasmid (31). Section 4 was combined with sections 1 and 2 by HiFi Assembly (NEB), generating an intermediate plasmid, and the overlaps between sections were verified by Sanger sequencing. Finally, the intermediate plasmid was digested with AvrII and assembled together with section 3 by HiFi Assembly to generate recoded MP6. The plasmid sequence was verified by NGS (see Data file S2 for sequence details).

### Automated Parallel Evolution and Strain Isolation

Two rounds of parallel mutagenesis and automated dynamic parallel selection were applied to Syn61 and later three rounds to Syn61Δ3. For mutagenesis, Syn61 was transformed with the mutagenesis plasmid MP6 (31) (or recoded MP6 for Syn61Δ3 and derivative evolutions) and the cells grown on LB agar containing 2% glucose and 20 μg/mL chloramphenicol. 96 individual colonies were picked into wells containing 200 μL of LB medium supplemented with 2% glucose and 20 μg/mL chloramphenicol and the colonies were grown overnight at 37° C., 220 rpm. The dense overnight cultures were diluted 1/400 into fresh LB medium supplemented with 20 μg/mL chloramphenicol and grown to OD₆₀₀˜0.1. Mutagenesis was induced by addition of L-arabinose to a final concentration of 0.5%, creating independent mutator cultures in each well. The mutator cultures were grown for 24 h at 37° C., 220 rpm. Following a 1/400 dilution step into fresh induction medium (LB with 20 μg/ml chloramphenicol and 0.5% L-arabinose), the cultures were grown for another 24 h at 37° C., 220 rpm. This equates to a growth phase of approximately 17 generations with induced mutagenesis (2×log₂(400)). In later rounds of evolution, the induction of mutagenesis was extended to up to 70 generations. Mutagenesis was induced for ˜17 generations in round 1 and 2 of Syn61 evolution. During Syn61Δ3 evolution mutagenesis was induced for ˜70 generations in round 1, ˜45 generations in round 2 and ˜60 generations in round 3.

Following mutagenesis, the mutator cultures were diluted 1/2 into a fresh 96-well plate with 200 μl LB medium supplemented with 2% glucose to suppress mutagenesis. The plate was transferred onto a robotics platform (Beckman Coulter Biomek FXp accessing Thermofisher Cytomat 2C incubator shaker and Molecular Devices SpectraMax 13 plate reader) for continuous growth (37° C., 400 rpm) and automated dynamic parallel selection for the fastest growing clones (scripts available at https://github.com/JWChin-Lab). The OD₆₀₀ was measured in each well at the start and then periodically again every 2-9 h, depending on the growth rate. When the OD₆₀₀ reached a threshold of 0.5 for at least 3 mutator wells, all cultures were diluted 1/400 into a fresh plate with 200 μl LB supplemented with 2% glucose. If no wells had reached the threshold, the three highest OD₆₀₀ values across the plate were used to estimate the next OD₆₀₀ measurement time point, i.e. the time required to reach the threshold. If fewer than 3 wells reached the threshold after three consecutive OD₆₀₀ measurements, the plate was incubated for another 12 h before a dilution step was implemented regardless of the measured cell density. Overall, this process flexibly adjusts the selective pressure in an effort to keep the fastest growing wells in exponential growth. After 6 dilution steps (equivalent to an estimated growth for 52 generations) the workflow was terminated and the growth rate was determined for all evolved mutator wells in the final plate as described below. The wells of evolved populations with the lowest doubling times were streaked out on LB agar plates with 2% glucose and 200 μg/ml streptomycin and incubated overnight at 37° C. to obtain single colonies. 4-12 individual evolved colonies were picked from each streaked population and growth rates measured for individual clones in direct comparison with the progenitor strain before evolution. The fastest growing clones were prepared for next-generation sequencing and further analysis as described below.

After one round of Syn61 evolution, cells from ten independent wells of the 96 well plate were streaked to obtain single colonies. 80 clones were picked from these streaks and their doubling time screened. 20 out of 80 screened clones isolated initially emerged with a reduced doubling time in single replicate experiments. However, only 4 of these clones showed improved growth in subsequent measurements that were performed in replicate. Two of these four clones (C02.5 and C02.6) are derived from a single well and the other two clones are both derived from a distinct single well (C04.4 and C04.7). All four clones were sequenced (see Data file S1 for a complete list of all accumulated mutations). One of these clones (C04.4) was carried forward and re-named Syn61(ev1).

After evolution of Syn61(ev1), cells from twelve independent wells of the 96 well plate were streaked to obtain single colonies. 88 clones were picked from these streaks and their doubling time screened. 18 out of 88 screened clones isolated initially emerged with a reduced doubling time in single replicate experiments. However, only 3 of these clones showed improved growth in subsequent measurements that were performed in replicate. Two of these clones (A1 1.2 and A1 1.3) are derived from a single well, and the third clone (E10.4) was from a distinct well. All three clones were sequenced (see Data file S1 for a complete list of all accumulated mutations). One of these clones (A1 1.2) was carried forward and re-named Syn61(ev2). Syn61(ev2) was used to generate Syn61Δ3.

Syn61Δ3 was evolved in two 96 well plates. Cells from 42 independent wells were steaked and a total of 184 clones were picked from these streaked cells and their doubling time screening (88 of the clones were from 22 wells in plate 1 and 96 clones were from 20 wells from plate 2). 130 out of the 184 clones stood out initially with reduced doubling time compared to Syn61Δ3. 22 of the fastest growing clones were characterised in (mostly) replicate growth experiments. These clones were derived from 11 independent wells (Plate 2: F11.2, F11.3, F11.5, F11.6, F11.7, F11.8, F11.9, F11.10 (all from well F11), E10.1, E5.1, F5.2, F8.1, F8.2, G5.2, A11.1; Plate 1: A7.4, D8.2, D8.4, A9.1, G8.1, D1.2, D11.1). The following clones were sequenced and selected for further sequencing analysis (see Data file S1 for a complete list of all accumulated mutations): A7.4, F11.9, E5.1, D8.2. One of these clones (F11.9) was carried forward and renamed Syn61Δ3 (ev3).

96 independent wells of Syn61Δ3 (ev3) were evolved. 19 wells with the lowest overall doubling time were streaked and 90 clones were isolated from 18 wells since one well did not survive the streak. 37 clones emerged with a reduced doubling time in single replicate measurements. 22 clones with the fastest growth were selected for replicate measurements and 8 clones from 5 distinct wells were identified to have improved growth compared to the progenitor: E7.4, E7.2 (from well E7); E10.3, E10.4 (from well E10); D5.3, D5.1 (from well D5); D8.1, B9.1. Four clones were sequenced and selected for further sequencing analysis: E7.4, E7.2, E10.4, D5.3 and D8.1 (Data file S1). One clone with the most improved growth (E7.4) was selected for the next round of evolution and renamed it Syn61Δ3 (ev4).

Syn61Δ3(ev4) was evolved in one 96-well plate (plate 1), with an adapted selection programme (with the maximum time for growth before the next dilution set to 6.5 h instead of 12 h) after the experiment did not yield any well with improved doubling time with the initial programme (12 h maximum time for growth before the next dilution). Also performed was a consecutive round of selection without intermediate mutagenesis on plate 1 and the resulting set of evolved pools are referred to as plate 2. Overall, fewer wells survived the adapted dynamic selection. 13 wells were streaked from plate 1 and 7 wells were streaked from plate 2 and a total of 152 clones were screened in single replicate measurements (96 from plate 1 and 56 from plate 2). 43 clones showed initial improved growth but only 5 clones remained with reproducibly faster growth in replicate measurements compared to the non-evolved progenitor, all from distinct wells: Plate 1: B12.3, D5.3, G12.6, E7.3; Plate 2: E8.4. One clone was selected as the most evolved Syn61 derivative used in this study (B12.3) and it was renamed Syn61Δ3 (ev5).

Deletion of prfA, serU and serT by Lambda-Red Recombination to Generate Syn61Δ3

A scarless deletion of serU, serT and prfA in Syn61(ev2) was performed, with each deletion proceeding through two sequential lambda-red mediated recombinations. In the first recombination a double selection marker cassette was provided to the recipient cell to replace the target gene. A repair template was provided in a second recombination to remove the double-selection marker and replace it with the native environment of the targeted locus, where only the sequence corresponding to the target gene was missing.

A recoded version of the rpsL-kan^{R} selection marker cassette (18) was used for all deletions. The recoded rpsL-kan^{R} was amplified with oligos containing ˜50 bp homology to the flanking genomic sequences of serU, serT and prfA. The repair templates for serU, serT and prfA were constructed through overlap extension PCR; the 500-600 bp regions directly upstream and downstream of the target gene were amplified from a Syn61 genomic DNA template with oligos designed to overlap by ˜40 bp. These two fragments were then combined into one PCR product by overlap extension PCR, and used it as a repair template. All oligonucleotide sequences are provided in Data file S2.

For the first recombination, Syn61(ev2) cells were transformed with the recoded helper plasmid. Single colonies were inoculated into 2×YT medium with 50 μg/ml apramycin and grown overnight at 37° C., shaking at 220 rpm. The overnight culture was diluted in 500 mL of 2×YT medium with 50 μg/ml apramycin to OD₆₀₀=0.05 and the cells grown at 37° C. with shaking until OD₆₀₀ 0.2. To induce lambda-red expression, L-arabinose powder was added to the culture to a final concentration of 0.5% w/v and incubated the culture for one additional hour at 37° C. with shaking. The cells were harvested at OD₆₀₀≈0.6, and the cells were made electrocompetent as described previously (32). 100 μL of electrocompetent cells were electroporated with ˜4 μg of rpsL-kan^{R} PCR product with homology to the serU locus, recovered for 1 hour in 4 mL SOB and then transferred to 100 mL 2×YT supplemented with 50 μg/ml apramycin. After 4 hours the cells were spun down and resuspended in 500 μL H₂O and plated in serial dilutions on 2×YT agar plates supplemented with 2% glucose, 50 μg/ml apramycin and 50 μg/ml kanamycin. The plates were incubated overnight at 37° C., and subsequently individual colonies were picked. These colonies were restreaked onto 2×YT agar plates supplemented with 2% glucose, 50 μg/ml apramycin and 50 μg/ml kanamycin. After overnight incubation at 37° C., the intended deletions were verified by colony PCR with primers flanking the locus of interest. This process was then repeated in the resulting strain, Syn61(ev2)ΔserU∴rpsL-kan^{R}, providing the serU repair template and counter-selecting on 2×YT agar plates supplemented with 2% glucose, 50 μg/ml apramycin and 200 μg/ml streptomycin. Removal of the rpsL-kan^{R} cassette was verified by colony PCR and Sanger sequencing. Sequential deletion of serT and prfA through an analogous 2-step process resulted in the creation of Syn61Δ3.

### Growth Rate Measurement and Analysis

Bacterial clones were grown overnight at 37° C. in LB supplemented with 200 μg/mL streptomycin. Overnight cultures were diluted 1:200 into 200 μL fresh medium in a 96-well plate. Growth was monitored on an Infinite 200 Pro plate reader (Tecan AG, Switzerland) at 37° C. with high speed linear shaking. Measurements of OD₆₀₀ were taken every 5 min for 18 h. To determine doubling times, the growth curves were log₂-transformed and the first derivative was determined (d(log₂(x))/dt) for all consecutive time-points. The inventors used the mean over ten consecutive time-points with the maximal log₂-derivative to calculate the doubling time (1/mean(max log₂-derivative)). Growth rates were calculated as the slope of a linear fit to the ten consecutive time-points with the maximal log₂-derivatives (as determined above). Curve fitting (linear regression) was performed on replicates simultaneously using Prism7 (Graphpad). Error bars show ±standard error of the fit.

To determine growth under standard laboratory conditions overnight cultures of Syn61Δ3(ev5) were diluted 1:100 into 80 ml fresh medium in a 250 ml conical flask. Three replicate cultures were incubated at 37° C. with shaking (220 rpm) and OD₆₀₀ measurements were taken every 10-30 min, every 10 min during exponential phase. For each independent culture the inventors log₂-transformed the growth curves and selected a 60 min window with the fastest growth during exponential phase. The growth rate (slope) and doubling time (1/slope) were calculated from a linear fit to log₂-derivatives of six consecutive time-points within the window. Curve fitting (linear regression) was performed for each replicate with Prism7 (Graphpad) and the average doubling time and growth rate was calculated. Error bars show ±standard error of the mean.

### Preparation of Whole-Genome Libraries for Next-Generation Sequencing

Colonies were picked from the clones of interest and used to inoculate 5 mL of LB supplemented with 200 μg/mL streptomycin, and incubated at 37° C. overnight with shaking at 220 rpm. Genomic DNA was extracted from these cultures with the DNEasy Blood and Tissue Kit (QIAgen), as per manufacturer's instructions. From purified genomic DNA, paired-end sequencing libraries were prepared with the Nextera XT DNA Library Preparation Kit (Illumina) by hand or with a Biomek FXp (Beckman Coulter, scripts available at https://github.com/JWChin-Lab), following the manufacturer's protocol but with reduced volumes: input gDNA (0.2 ng/μL, 1 μL), TD Buffer (2 μL), ATM (1 μL), NT Buffer (1 μL), indeces (1 μL), NPM (3 μL). Index sequences were generated from the ‘Illumina Adapter Sequences’ support document (Nextera DNA indexes, pg 16, dated June 2020), purchased from Biomers and used at 10 μM Libraries were then purified with AMPure XP magnetic beads (Beckman Coulter) as per manufacturer's instructions (6:10 bead:reaction vol. ratio). Libraries were quantified by Qubit (Thermofisher) and subsequently pooled, denatured and paired-end sequenced on a MiSeq (Illumina) according to manufacturer's instructions using v3 MiSeq reagent kits of either 600 cycles (2×300 cycles) or 150 cycles (2×75 cycles).

### Analysis of Syn61 Knockout and Evolved Strains

To analyse the genome sequences of the Syn61 derivatives generated in this work the inventors used breseq, a software package that aligns next generation sequencing reads (generated as above) to a reference genome and identifies mutations, deletions and insertions (33). The program was used to analyse isolated clones after each round of evolution or targeted gene knockout (serT, serU, prfA), to identify the mutations that arose during the generation of these strains (Data file S1 contains a list of all intermediate strains and mutations). Additionally, a previously described workflow (32) (script available at https://github.com/TiongSun/iSeq) was used to align reads to a reference Syn61 genome sequence and visualised read alignments in the Integrative Genomics Viewer (34). Further, the inventors identified ORFs with more than one mutation (Fig. S2). Since clones derived from the same evolved pool were found to be nearly identical in their sequence the inventors selected only one clone per pool for the analysis, one with the most overall mutations. The inventors compared all mutations in ORFs accumulated during evolution before the deletion of serT, serU and prfA separately from mutations gained in the evolution of Syn61Δ3 due to alteration of the genetic background upon removal of the decoding elements. Under the assumption that mutations are independent events the inventors calculated the probability for at least two mutations arising by chance in one ORF by

$$\\ p = 1 - \frac{a !}{{a}^{b} ( a - b ) !} ,$$

where a is the total number of ORFs (a=3556 in Syn61) and b is the average number of mutations accumulated in ORFs after one round of evolution.

### Example 2—tRNA Deletion Ablates Virus Production in Syn61Δ3

The inventors investigated the effects of deleting the genes encoding tRNA^{Ser}_{CGA} and tRNA^{Ser}_{UGA} and RF-1 on phage propagation by Syn61Δ3 (FIG. 2A), in a modified one-step growth experiment (24).

For Syn61(ev2) the total titer of phage T6 (a representative of the lytic, T-even family (FIG. 2B)) briefly dropped (as phage infect cells) before rising to 2-logs₁₀ above the input titer, as infected cells produced new phage particles (FIG. 2C, Fig. S3A). As expected, the OD₆₀₀ of Syn61(ev2) was decreased by infection with T6 phage, which is lytic (FIG. 2D). Syn61ΔRF-1 (Data file S1) and Syn61(ev2) produced a comparable level of phage on a comparable timescale, and showed similar changes in OD₆₀₀ upon infection. The inventors conclude that deletion of RF-1 alone has little, if any, effect on T6 phage production or cell lysis.

Infection of Syn61Δ3 with T6 phage led to a steady decrease in total phage titer. Importantly, this decrease was comparable to that observed when protein synthesis—and therefore phage production in cells—was completely inhibited by addition of gentamicin (FIG. 2C, Fig. S3B). Moreover, T6 infection had a minimal effect on the growth of Syn61Δ3 (FIG. 2D). The inventors conclude that Syn61Δ3 does not produce new phage particles upon infection with T6 phage and that T6 phage does not lyse these cells. Similar results were obtained with T7 phage, which has 57 TCG codons, 114 TCA codons and 6 TAG codons in its 40 kb genome (Fig. S3A, C and D). The inventors treated cells with a cocktail of phage containing lambda, P1vir, T4, T6 and T7, which have TCA or TCG sense codons 10- to 58-times more abundant than the amber stop codon in their genomes (FIG. 2E, Fig. S3E), and found that the treatment with this phage cocktail led to lysis of Syn61(ev2) and Syn61ΔRF-1, but had little effect on the growth of Syn61Δ3 (FIGS. 2, F and G), suggesting that the deletion of tRNAs in Syn61Δ3 provides resistance to a broad range of phage.

### Materials and Methods for Example 2

### General Phage Methods

Phage λ, P1vir, T4, and T6 were provided by George Salmond (University of Cambridge, Department of Biochemistry). Phage T7 was obtained from ATCC (BAA-1025-B2). The titer of each phage stock was estimated by serially diluting stocks in phage buffer (1 mM MgSO4, 1 mM Tris-HCl, 0.1% gelatine), then infecting E. coli MG1655 in top agar (LB agar with 0.35% agar), and allowing plaques to mature at 37° C. overnight. The plaques were counted and the PFU/mL was calculated for each stock.

### Total Titer of Infecting Phage

Overnight cultures of E. coli Syn61 variants were diluted to OD₆₀₀=0.5 in 2×YT, and then transferred 3 mL to 50 mL Falcon tubes and infected them with phage T6 or T7 (MOI=5·10⁻² and 5·10⁻⁵, respectively). For each phage a cell-free control was included, where phage was added to 3 mL 2×YT. The infected cultures were incubated at 37° C. for 4 hours, shaking at 220 rpm. At relevant timepoints 100 μL of sample was transferred from each culture to a microcentrifuge tube on ice, containing 400 μL phage buffer and 50 μL chloroform. Samples were immediately vortexed thoroughly, centrifuged at 13000×g for 4 min, transferred to a LoBind microcentrifuge tube (Eppendorf) and stored at 4° C. To quantify phage titer, each sample was serially diluted in phage buffer. 10 μL of the relevant dilution was used to infect a 200 μL overnight culture of E. coli MG1655 grown in 2×YT. Infected cells were mixed with 4 mL molten top-agar and poured onto a TYE agar plate. After drying, plates were incubated at 37° C. overnight for bacterial lawns to grow and phage plaques to develop. The number of plaques were counted and multiplied by the dilution in order to calculate the phage titer of the original sample.

As a background control, E. coli Syn61 was rendered metabolically inactive: the inventors diluted an overnight culture to OD₆₀₀=1 in 10 mL 2×YT, added gentamycin to a final concentration of 1000 μg/mL, and incubated the cells at 37° C. for 6 hours, shaking at 220 rpm. The cells were centrifuged at 4000×g for 10 mins and then the supernatant, which contained the antibiotic, was aspirated. The inactive cells were resuspended in 2×YT to OD₆₀₀=0.5 and infected them with phage as above. The inventors assessed the metabolic activity of the gentamycin-treated cells by 35S incorporation into newly synthesized proteins. 1 mL of gentamycin-treated E. coli Syn61 culture (OD₆₀₀=0.5) was incubated for 1 hour at 37° C. in the presence of 1.5 μL 35S labelled mixture of methionine and cysteine (11 mCi/mL, 1175 Ci/mmol, Perkin Elmer NEG072).

The inventors measured the OD₆₀₀ at the end of the incubation, spun down the cells at 4000×g for 10 min washed the cells with cold PBS. Based on the OD₆₀₀ measurement above, the cells were resuspended in 1× BugBuster Protein Extraction Reagent (Merck) to a density of 1.4×10⁹ cells/mL (assuming that OD₆₀₀ of 1.0=1×10⁹ cells/mL with 1 cm pathlength). Cells were lysed for 1 hour and the equivalent of 2.8×10⁷ cells were loaded on each lane of a 4-12% Bis-tris polyacrylamide gel. The inventors then analysed the lysates by SDS-PAGE, following by staining with Quick Coomassie Stain (Neo Biotech) as per manufacturer's instructions. The resulting gels were dried and exposed to a storage phosphor screen (GE Healthcare Life Sciences) for 2 weeks, followed by imaging on an Amersham Typhoon (GE Healthcare Life Sciences).

### Phage Infection Assays

For simultaneous infection by multiple phage, the inventors prepared cultures of Syn61 variants as above and added a cocktail of all five phage (λ, P1vir, T4, T6, and T7, MOI=1·10⁻² for each phage). Cell growth and lysis was monitored by measuring OD₆₀₀ after 4 hours and comparing to uninfected cultures.

### Phage Genome TCG, TCA, TAG Codon Counting

The inventors identified the number and location of the TCG, TCA and TAG codons in the genome of different phage using a custom Python 3.7 script. Briefly, the script opens a GenBank file containing the annotated genome of the phage of interest using Biopython (35), then scans coding sequences to identify target codons and their location, and stores them in memory. The Python library matplotlib (36) is then used to generate a plot of the genome where the location of each target codon is represented with a vertical line. The script is available at https://github.com/JWChin-Lab.

### Example 3—ncAA Incorporation

Reassigning Target Codons for ncAA Incorporation

The inventors expressed Ub_{11XXX} genes (ubiquitin-His₆ bearing TCG, TCA or TAG at position 11), and genes encoding the cognate orthogonal MmPylRS/MmtRNA^{Pyl}_{YYY} pair (25) (in which the anticodon is complementary to the codon at position 11 in the Ub gene) in Syn61Δ3(ev5) (FIG. 3A, Data file S2).

In the absence of added ncAA, little to no ubiquitin was detected from Ub genes bearing a target codon at position 11, while control experiments demonstrated that ubiquitin is produced from a ‘wildtype’ gene that does not contain any target codons (FIG. 3B). Thus, none of the target codons are read by the endogenous translational machinery in Syn61Δ3. This further demonstrates that all of the target codons are orthogonal in this strain.

Upon addition of a ncAA substrate for the MmPylRS/MmtRNA^{Pyl} pair (N^{ε}-((tert-butoxy)carbonyl)-L-lysine (BocK)) (25), ubiquitin was produced at levels comparable to wildtype controls (FIG. 3B, Data file S4). ESI-MS and MS/MS demonstrated the genetically directed incorporation of BocK at position 11 of Ub in response to each target codon using the complementary MmPylRS/MmtRNA^{Pyl}_{YYY} pair (FIG. 3C, Fig. S4A). Additional experiments demonstrated efficient incorporation of ncAAs in response to sense and stop codons in GST-MBP (Fig. S5, Data file S4). The inventors demonstrated good yields of Ub-His₆ incorporating 2, 3, or 4 ncAAs into a single polypeptide in response to each of the target codons (Data file S4, FIG. 3, D to I, and Fig. S4, B to G), and further demonstrated the incorporation of 9 ncAAs in response to 9 TCG codons in a single repeat protein (Fig. S6). Together, these results demonstrated that the sense codons TCG and TCA, and the stop codon TAG, can be efficiently reassigned to ncAAs in Syn61Δ3 derivatives.

Encoding Distinct ncAAs in Response to Distinct Target Codons

Next, the inventors assigned TCG, TCA and TAG codons to distinct ncAAs in Syn61Δ3(ev4) using engineered mutually orthogonal aaRS/tRNA pairs that recognize distinct ncAAs and decode distinct codons (FIG. 4A, Fig. S7). The inventors incorporated two distinct ncAAs into ubiquitin in response to TCG and TAG codons (FIG. 4B, Fig. S8A to B), and demonstrated the incorporation of two distinct ncAAs at four sites in ubiquitin, with each ncAA incorporated at two different sites in the protein (FIGS. 4B and C, and Fig. S8C to E, and Data file S4). The inventors incorporated three distinct ncAAs into ubiquitin, in response to TCG, TCA and TAG codons (FIG. 4D, E, and Fig. S8F). The inventors demonstrated the generality of their approach by synthesizing seven distinct versions of ubiquitin, each of which incorporated three distinct ncAAs (Fig. S9, Fig. S10, Data file S4)

### Encoded Non-Canonical Polymers and Macrocycles

For a linear polymer composed of two distinct monomers (A and B) there are four elementary polymerization steps (A+B->AB, B+A->BA, A+A->AA, B+B->BB) from which any sequence can be composed (FIG. 5A). For ribosome-mediated polymerization these four elementary steps correspond to each monomer acting as an A-site or P-site substrate to form a bond with another copy of the same monomer or with a distinct monomer (FIG. 5A). The inventors encoded each elementary step by inserting TCG-TCG (encoding AA; monomer A was arbitrarily assigned to the TCG codon in this nomenclature), TAG-TAG (encoding BB; monomer B was assigned to the TAG codon), TCG-TAG (encoding AB) and TAG-TCG (encoding BA) at codon 3 of a sfGFP gene. The inventors demonstrated the elementary steps for three pairs of monomers: A=BocK, B=p-IPhe; A=CbzK, B=p-I-Phe; A=NE-allyloxycarbonyl-L-lysine (AllocK), B=CbzK (FIG. 5B, Fig. S11). The inventors genetically encoded six entirely non-natural tetrameric sequences and a hexameric sequence for each pair of monomers, as well as an octameric sequence for the AllocK, CbzK pair (22 synthetic polymer sequences in total) (Fig. S11, FIGS. 5, C, D, and E, Fig. S12). All encoded polymerizations were ncAA dependent (Fig. S11, FIGS. 5, C, D and E, Fig. S12B) and ESI-MS confirmed that the inventors had synthesized the non-canonical hexamers and octamers as sfGFP fusions (FIG. 5F, Fig. S12C). The inventors encoded tetramer and hexamer sequences composed of AllocK and CbzK between SUMO and GyrA-CBD and purified the free polymers (FIGS. 5, G, H, and I, Fig. S13). Finally, the inventors encoded the synthesis of a non-natural macrocycle reminiscent of the products of non-ribosomal peptide synthetases (FIGS. 5, G and J).

TABLE 1
(“Data file S4”) - Expression Yields

| | | | | Fraction | | |
|---|---|---|---|---|---|---|
| | | | | Full- | Yield | % of |
| Reporter | aaRS/tRNA | ncAA | Cells | length | (mg/L) | WT |
| Wildtype | MmPylRS/ | (—) | Syn61Δ3 | | 9.7 | 100.0 |
| Ubiquitin | MmtRNAPyl(CGA) | | (ev5) | | | |
| Wildtype | MmPylRS/ | (—) | MDS42 | | 10.4 | 107.2 |
| Ubiquitin | MmtRNAPyl(CGA) | | | | | |
| Single Ub- | MmPylRS/ | Bock | Syn61Δ3 | | 10.8 | 111.7 |
| 11TCG | MmtRNAPyl(CGA) | (5 mM) | (ev5) | | | |
| Single Ub- | MmPylRS/ | Bock | Syn61Δ3 | | 8.6 | 88.4 |
| 11TCA | MmtRNAPyl(UGA) | (5 mM) | (ev5) | | | |
| Single Ub- | MmPylRS/ | Bock | Syn61Δ3 | | 8.3 | 85.9 |
| 11TAG | MmtRNAPyl(CUA) | (5 mM) | (ev5) | | | |
| Double Ub- | MmPylRS/ | Bock | Syn61Δ3 | | 14.1 | 145.5 |
| 11TCG/65TCG | MmtRNAPyl(CGA) | (5 mM) | (ev5) | | | |
| Double Ub- | MmPylRS/ | Bock | Syn61Δ3 | | 8.3 | 85.8 |
| 11TCA/65TCA | MmtRNAPyl(UGA) | (5 mM) | (ev5) | | | |
| Double Ub- | MmPylRS/ | Bock | Syn61Δ3 | | 9.0 | 92.9 |
| 11TAG/65TAG | MmtRNAPyl(CUA) | (5 mM) | (ev5) | | | |
| Triple Ub- | MmPylRS/ | Bock | Syn61Δ3 | | 10.6 | 109.1 |
| 11TCG/14TCG/ | MmtRNAPyl(CGA) | (5 mM) | (ev5) | | | |
| 65TCG | | | | | | |
| Triple Ub- | MmPylRS/ | Bock | Syn61Δ3 | | 5.8 | 59.5 |
| 11TCA/14TCA/ | MmtRNAPyl(UGA) | (5 mM) | (ev5) | | | |
| 65TCA | | | | | | |
| Triple Ub- | MmPylRS/ | Bock | Syn61Δ3 | | 1.5 | 15.1 |
| 11TAG/14TAG/ | MmtRNAPyl(CUA) | (5 mM) | (ev5) | | | |
| 65TAG | | | | | | |
| Quadruple Ub- | MmPylRS/ | Bock | Syn61Δ3 | | 2.6 | 27.1 |
| 9TCG/11TCG/ | MmtRNAPyl(CGA) | (5 mM) | (ev5) | | | |
| 14TCG/65TCG | | | | | | |
| Quadruple Ub- | MmPylRS/ | Bock | Syn61Δ3 | | 2.1 | 21.8 |
| 9TCA/11TCA/ | MmtRNAPyl(UGA) | (5 mM) | (ev5) | | | |
| 14TCA/65TCA | | | | | | |
| Quadruple Ub- | MmPylRS/ | Bock | Syn61Δ3 | | 2.8 | 28.9 |
| 9TAG/11TAG/ | MmtRNAPyl(CUA) | (5 mM) | (ev5) | | | |
| 14TAG/65TAG | | | | | | |
| | | | | | | 0.0 |
| Double Ub- | | CbzK | Syn61Δ3 | | 7.4 | 76.7 |
| 11TCG/65TAG | | (2 mM)/ | (ev5) | | | |
| | | p-I-Phe | | | | |
| | | (2 mM) | | | | |
| Double-Double | | CbzK | Syn61Δ3 | | 1.6 | 16.7 |
| Ub- 11TCG/ | | (2 mM)/ | (ev5) | | | |
| 14TCG/57TAG/ | | p-I-Phe | | | | |
| 65TAG | | (2 mM) | | | | |
| Wildtype | 1R26PylRS/ | CbzK | Syn61Δ3 | | 7.7 | 79.5 |
| Ubiquitin | AlvtRNAΔNPyl | (2 mM)/ | (ev5) | | | |
| | (8)(CGA); AfTyrRS(p-I- | p-I-Phe | | | | |
| | Phe)/AftRNATyr | (2 mM)/ | | | | |
| | (A01)(CUA); MmPylRS/ | Bock | | | | |
| | MmtRNAPyl(UGA) | (5 mM) | | | | |
| Triple Ub-9TAG/ | 1R26PylRS/ | CbzK | Syn61Δ3 | | 2.8 | 28.7 |
| 11TCG/14TCA | AlvtRNAΔNPyl | (2 mM)/ | (ev5) | | | |
| | (8)(CGA); AfTyrRS(p-I- | p-I-Phe | | | | |
| | Phe)/AftRNATyr | (2 mM)/ | | | | |
| | (A01)(CUA); MmPylRS/ | Bock | | | | |
| | MmtRNAPyl(UGA) | (5 mM) | | | | |
| Triple Ub-9TAG/ | 1R26PylRS/ | CbzK | Syn61Δ3 | | 3.1 | 31.7 |
| 11TCG/14TCA | AlvtRNAΔNPyl | (2 mM)/ | (ev5) | | | |
| | (8)(CGA); AfTyrRS(p-I- | p-I-Phe | | | | |
| | Phe)/AftRNATyr | (2 mM)/ | | | | |
| | (A01)(CUA); MmPylRS/ | Allock | | | | |
| | MmtRNAPyl(UGA) | (5 mM) | | | | |
| Triple Ub-9TAG/ | 1R26PylRS/ | CbzK | Syn61Δ3 | | 3.5 | 35.9 |
| 11TCG/14TCA | AlVARNAΔNPyl | (2 mM)/ | (ev5) | | | |
| | (8)(CGA); AfTyrRS(p-I- | p-I-Phe | | | | |
| | Phe)/AftRNATyr | (2 mM)/ | | | | |
| | (A01)(CUA); MmPylRS/ | CypK | | | | |
| | MmtRNAPyl(UGA) | (5 mM) | | | | |
| Triple Ub- | 1R26PylRS/ | CbzK | Syn61Δ3 | | 5.9 | 60.8 |
| 9TAG/11TCG/ | AlvtRNAΔNPyl | (2 mM)/ | (ev5) | | | |
| 14TCA | (8)(CGA); AfTyrRS(p-I- | p-I-Phe | | | | |
| | Phe)/AftRNATyr | (2 mM)/ | | | | |
| | (A01)(CUA); MmPylRS/ | AlkK | | | | |
| | MmtRNAPyl(UGA) | (5 mM) | | | | |
| Triple Ub-9TAG/ | MmPylRS/ | Allock | Syn61Δ3 | | 3.0 | 31.0 |
| 11TCG/14TCA | MmtRNAPyl(UGA); | (2 mM)/ | (ev5) | | | |
| | MjTyrRS(p-Az-Phe)/ | CbzK | | | | |
| | MjtRNATyr(CUA); | (2 mM)/ | | | | |
| | 1R26PylRS/ | p-Az-Phe | | | | |
| | AlvtRNAΔNPyl | (2 mM) | | | | |
| | (8)(UGA) | | | | | |
| Triple Ub-9TAG/ | MmPylRS/ | AlkK | Syn61Δ3 | | 0.9 | 9.1 |
| 11TCG/14TCA | MmtRNAPyl(UGA); | (2 mM)/ | (ev5) | | | |
| | MjTyrRS(p-Az-Phe)/ | CbzK | | | | |
| | MjtRNATyr(CUA); | (2 mM)/ | | | | |
| | 1R26PylRS/ | p-Az-Phe | | | | |
| | AlvtRNAΔNPyl | (2 mM) | | | | |
| | (8)(UGA) | | | | | |
| Triple Ub-9TAG/ | MmPylRS/ | Allock | Syn61Δ3 | | 3.8 | 38.7 |
| 11TCG/14TCA | MmtRNAPyl(UGA); | (2 mM)/ | (ev5) | | | |
| | MjTyrRS(3-Nitro-Tyr)/ | CbzK | | | | |
| | MjtRNATyr(CUA); | (2 mM)/ | | | | |
| | 1R26PylRS/ | 3-Nitro-Tyr | | | | |
| | AlvtRNAΔNPyl | (2 mM) | | | | |
| | (8)(UGA) | | | | | |
| Wildtype GST- | MmPylRS/ | (—) | Syn61Δ3 | 0.92 | 23.1 | 100.1 |
| MBP | MmtRNAPyl(CGA) | | (ev5) | | | |
| GST-TCG-MBP | MmPylRS/ | Bock | Syn61Δ3 | 0.86 | 21.6 | 93.7 |
| | MmtRNAPyl(CGA) | (5 mM) | (ev5) | | | |
| GST-TAG-MBP | MmPylRS/ | Bock | Syn61Δ3 | 0.82 | 17.6 | 76.3 |
| | MmtRNAPyl(CUA) | (5 mM) | (ev5) | | | |
| Wildtype GST- | MmPylRS/ | (—) | MDS42 | 0.96 | 24.8 | 107.4 |
| MBP | MmtRNAPyl(CGA) | | | | | |
| GST-TCG-MBP | MmPylRS/ | (—) | MDS42 | 0.93 | 21.3 | 92.0 |
| | MmtRNAPyl(CGA) | | | | | |
| GST-TAG-MBP | MmPylRS/ | Bock | MDS42 | 0.44 | 6.1 | 26.4 |
| | MmtRNAPyl(CUA) | (5 mM) | | | | |
| SUMO- | MmPylRS/ | (—) | Syn61Δ3 | | 68.8 | 100.0 |
| GSGSGSGS- | MmtRNAPyl(CGA); | | (ev5) | | | |
| GyrA- CBD | 1R26PylRS/ | | | | | |
| | AlvtRNAΔNPyl | | | | | |
| | (8)(CUA) | | | | | |
| SUMO-TCG/ | MmPylRS/ | Allock | Syn61Δ3 | | 38.8 | 56.4 |
| TAG/TCG/TAG- | MmtRNAPyl(CGA); | (2 mM)/ | (ev5) | | | |
| GyrA- CBD | 1R26PylRS/ | CbzK | | | | |
| | AlvtRNAΔNPyl | (2 mM) | | | | |
| | (8)(CUA) | | | | | |
| SUMO-TCG/ | MmPylRS/ | Allock | Syn61Δ3 | | 13.6 | 19.8 |
| TAG/TCG/ | MmtRNAPyl(CGA); | (2 mM)/ | (ev5) | | | |
| TAG/TCG/ | 1R26PylRS/ | CbzK | | | | |
| TAG- GyrA- | AlvtRNAΔNPyl | (2 mM) | | | | |
| CBD | (8)(CUA) | | | | | |
| SUMO- | MmPylRS/ | (—) | Syn61Δ3 | | 40.4 | 100.0 |
| CGSGSGSGS - | MmtRNAPyl(CGA); | | (ev5) | | | |
| GyrA-CBD | 1R26PylRS/ | | | | | |
| | AlvtRNAΔNPyl | | | | | |
| | (8)(CUA) | | | | | |
| SUMO-C-TCG/ | MmPylRS/ | Allock | Syn61Δ3 | | 29.2 | 72.3 |
| TAG/TCG/ | MmtRNAPyl(CGA); | (2 mM)/ | (ev5) | | | |
| TAG-GyrA- CBD | 1R26PylRS/ | CbzK | | | | |
| | AlvtRNAΔNPyl | (2 mM) | | | | |
| | (8)(CUA) | | | | | |

### Materials and Methods for Example 3

### Construction of Vectors for Expressing Ubiquitin and GFP

All cloning was conducted in E. coli DH10b. The plasmids for expressing ubiquitin (Ub) or sfGFP were constructed based upon a pBAD expression system (37). Recoded wild-type ubiquitin, sfGFP, apra^{R}, and araC were ordered as gBlocks (IDT). The plasmids were then constructed through a 4-piece assembly with HiFi DNA Assembly Mix (NEB), where the overlapping fragments were i) either recoded wild-type Ub or sfGFP, ii) recoded apra^{R}, iii) recoded araC, and iv) the pBAD vector origin region, amplified by PCR. This generated plasmids pBAD_Ub-wt_rec and pBAD_sfGFP-wt_rec. Variants of these plasmids where TCG, TCA or TAG codons were introduced into the Ub or sfGFP genes were generated by site-directed mutagenesis of pBAD_Ub-wt_rec and pBAD_sfGFP-wt_rec. See Data file S2 for sequence details of all plasmids.

Construction of aaRS/tRNA Plasmids for Decoding TCG, TCA, or TAG Codons

All cloning was conducted in E. coli DH10b. For incorporating one type of ncAA, the inventors used a pMB1-based plasmid encoding the aaRS/tRNA pair that directs ncAA incorporation. For tRNA^{Pyl} constructs with anticodons CGA and UGA, the inventors designed pylT tRNA genes (including pylTopt mutations from ref (38)) such that the entire anticodon stem loop (ASL—the anticodon plus 6 nucleotides on either side) was replaced by the ASL sequence of serU (in the case of CGA) or serT (in the case of UGA) E. coli genes.

pMB1-based aaRS/tRNA plasmids were constructed by HiFi Assembly of multiple fragments, including i) a recoded aaRS under control of the constitutive glnS promoter, synthesized as a gBlock (IDT), ii) a tRNA gene placed in between an lpp promoter and an rrnC terminator, synthesized as a gBlock (IDT), iii) a recoded kan^{R} gene, and iv) a pNMB1 vector origin amplified by PCR from a previously described pMB1-based plasmid (37). With this strategy the plasmids pMB1_MmPylRS_tRNA-Pyl-opt(CGA/UGA/CUA) were assembled. All plasmid sequences are available via their Genbank accessions listed in Data file S2.

Design and Construction of Plasmids Encoding aaRS/tRNA Pairs for Double or Triple ncAA Incorporations

For double and triple distinct ncAA incorporation experiments, the inventors designed plasmids polycistronically encoding two aaRS/tRNA pairs (16). The inventors placed the two aaRS CDSs downstream of a glnS promoter; the intergenic region between the aaRS genes was optimized with an RBS calculator program (https://salislab.net/software/). Both tRNA genes were placed downstream of a lpp promoter. The intergenic regions between tRNA genes were based upon the intergenic regions between the alaX and alaW genes in the E. coli genome.

To construct the plasmids encoding two aaRS/tRNA pairs, the inventors ordered recoded aaRS genes preceded by their corresponding RBS sequences as individual gBlocks (IDT). The polycistronic tRNAs, along with the lpp promoter, rrnC terminator and intergenic region, were ordered as a single gBlock (IDT). The inventors assembled synthetic DNA fragments encoding aaRS genes and tRNAs together with a pMB1 backbone containing kan^{R} (described above) by HiFi Assembly (NEB). This way the inventors generated plasmids pMB1_1R26PylRS(CbzK)_AfTyrRS(p-I-Phe)_AlvtRNA-ΔNPyl(8)(CGA)_AftRNA-Tyr(A01)(CUA), pMB1_MmPylRS_1R26PylRS(CbzK)_MmtRNAPyl-opt(CGA)_AlvtRNA-ΔNPyl(8)(CUA), pMB1_MmPylRS_AfTyrRS(p-I-Phe)_MmtRNA-Pylopt(CGA)-AftRNA-Tyr(A01)(CUA) and pMB1_MmPylRS_IR26PylRS(CbzK)_MmtRNA-Pylopt(CGA)_AlvtRNA-ΔNPyl(8)(UGA). Plasmid sequences are available via their Genbank accessions listed in Data file S2.

For incorporating three distinct ncAAs, the inventors used pNMB1_MmPylRS_tRNA-Pyl-opt(UGA) as a template to PCR-amplify a DNA fragment encompassing i) MmPylRS under control of a glnS promoter and ii) a pylT-serT(UGA) chimeric tRNA gene (see above) between an lpp promoter and an rrnC terminator. The inventors then used site-directed mutagenesis to separately created an intermediate pBAD_Ub derivative where codon positions 9, 11 and 14 of ubiquitin are substituted with TAG, TCG and TCA respectively. Next, the MmPylRS/pylTserT(UGA) fragment was inserted downstream of the ubiquitin gene in the intermediate plasmid by HiFi Assembly. Finally, site-directed mutagenesis was performed in the resulting plasmid with primers that directed the introduction of ‘opt’ mutations into the sequence of tRNA^{Pyl} as in ref (38), to generate pBAD_Ub9TAG11TCG14TCA-MmPylRS_MmtRNA-Pyl-opt(UGA). The inventors used this plasmid as a starting point to create pBAD_Ub9TAG11TCG14TCA-MjTyrRS(p-Az-Phe)_tRNA-Mj-Tyr(CUA) and pBAD_Ub9TAG11TCG14TCA-MjTyrRS(3-Nitro-TyrRS)_tRNA-Mj-Tyr(CUA), by replacing the aaRS/tRNA pair of the parent plasmid with recoded synthetic DNA fragments of MjTyrRS(p-Az-Phe) or MjTyrRS(3-Nitro-TyrRS), or their corresponding tRNAs, by HiFi assembly. The sequences are detailed in Data file S2.

Experiments describing the incorporation of BocK and p-I-Phe into sfGFP (FIGS. 5C and F) used derivatives of pBAD_sfGFP_MmPylRS_MmtRNA-Pyl-opt(CGA). Starting from pBAD_Ub9TAG11TCG14TCA-MmPylRS_MmtRNA-Pyl-opt(UGA), the inventors replaced the Ub gene with wild-type sfGFP by HiFi Assembly. The inventors subsequently derivatized position 3 of sfGFP by introducing the different codon combinations by site-directed mutagenesis. Finally, site-directed mutagenesis was used to replace the ASL in each of the resulting plasmids from serT to serU, generating a set of pBAD_sfGFP_MmPylRS_MmtRNA-Pyl-opt(CGA) derivatives (see Data file S2 for the full list of plasmids).

### Western Blot Analyses of Syn61 Expressions of Ubiquitin Containing Target Codons

For expressing ubiquitin variants, the inventors co-transformed 60 μL of electrocompetent Syn61Δ3, Syn61Δ3(ev3) or Syn61Δ3(ev4) cells with a pMB1_MmPylRS_tRNA-Pyl-opt(YYY) plasmid (with either serU(CGA), serT(UGA) or CUA anticodon, see Data file S2) and the relevant pBAD_Ub plasmid (1 μL per plasmid). Electroporated cells were recovered in 1 mL of prewarmed SOB at 37° C. while shaking at 1000 rpm on a benchtop thermomixer (Eppendorf) for 1 h 30 min. Following recovery, 1 mL of cells were diluted into 6 mL of pre-warmed 2×YT containing 50 μg/mL kanamycin and 50 μg/mL apramycin. The diluted cells were then incubated for 18 h at 37° C. while shaking at 220 rpm. Expression of ubiquitin was initiated by diluting the overnight culture 1:100 into 5-50 mL of pre-warmed 2×YT containing 50 μg/mL kanamycin, 50 μg/mL apramycin, 0.2% L-arabinose (w/v), and either 0 mM or 5 mM (BocK). After 8 hours of expression at 37° C. with shaking at 220 rpm, the cells were harvested by centrifugation (4,000×g 20 min), and the pellet was frozen at −20° C. until further use. For western blot analyses post-expression, the inventors harvested 5×10⁷ cells (assuming that OD₆₀₀ of 1.0=1·10⁹ cells/mL with 1 cm pathlength) by centrifugation (10000×g, 1 min) in a 1.5 mL microcentrifuge tube. The cell pellet was resuspended in 50 μL of 1×LDS sample buffer (BioRad) with 5% β-mercaptoethanol, boiled at 95° C. for 5 min, and then vortexed for 5 min. 10 μL of each sample was loaded into a 4-12% Bis-Tris NuPAGE SDS-PAGE gel (Invitrogen) and electrophoresed for 45 min at 180 V. Transfer to a PVDF membrane was accomplished with an iBlot 2 semi-wet transfer system. The membrane was then blocked for 30 min with 5 mL of Odyssey Blocking Buffer, followed by incubation with 5 mL of anti-His-tag primary antibody (Abcam, cat. no. ab18184) diluted 1:1000 in blocking buffer with 0.2% Tween-20. After 1 hour at room temperature, the membrane was washed three times for 5 min each with 5 mL of PBS containing 0.2% Tween-20. The secondary antibody was added at 1:15000 dilution in a 5 mL volume, diluted into a 50/50 mix of blocking buffer and 0.2% Tween-20, supplemented with 0.01% SDS. The secondary antibody (IRDye 925-68070, LiCor) was incubated for 30 min at room temperature, followed by three washes of 5 min each with 5 mL of PBS containing 0.2% Tween-20. Imaging of western blots was performed on a Typhoon Trio phosphorimager (GE Life Sciences).

### GFP Expression Measurements

The inventors performed expression of recoded sfGFP genes bearing single or multiple TCG, TCA, or TAG codons in the same media conditions as the expressions from Ubiquitin genes (see section above). Briefly, 50 μL of cells (Syn61Δ3(ev4)) were co-electroporated with both a pMVB1-based aaRS/tRNA plasmid (1 μL) as well as a pBAD_sfGFP reporter plasmid (1 μL). The transformed cells were recovered for 1 h 30 min in 1 mL of SOB while shaking at 1000 rpm at 37° C., and subsequently the entire 1 mL of cells were inoculated into 6 mL of 2×YT containing 50 μg/mL kanamycin and 50 μg/mL apramycin and incubated overnight at 37° C. while shaking at 220 rpm. The inventors performed expressions in 96-well microtiter plate format, inoculating overnight cultures 1:100 into 0.5 mL of 2×YT containing kanamycin (50 μg/mL), apramycin (50 μg/mL), Larabinose (0.2%), and the presence or absence of 2 mM ncAA. The 96-well plates were incubated for 16 hours at 37° C. while shaking at 750 rpm in a Thermo-Shaker (Grant-bio). After centrifugation for 10 min at 3200 g, cell pellets were resuspended in 150 μL of PBS. To measure GFP expression normalized by cell density, 100 μL of resuspended cells was transferred to a Costar clear 96-well flat-bottom plate, and OD₆₀₀ and GFP fluorescence (λₑₓ: 485 nm; λₑₘ: 520 nm) measurements were recorded on a PHERAstar FS plate reader (BMG LABTECH) with the gain set to 200 and the focal adjustment at 3.5 mm.

Purification of His6x-Tagged Reporter Proteins from E. coli

Cells (Syn61Δ3(ev4) or Syn61Δ3(ev5)) harbouring i) a pMB1-based plasmid carrying aaRS/tRNA pairs and ii) a pBAD_sfGFP or pBAD_Ub derivative were grown overnight at 37° C. with shaking at 220 rpm. Overnight cultures were prepared in 10 mL volumes for reporters containing a single target codon and larger volumes (20-50 mL) for those containing multiple codons. Expression cultures were centrifuged for 10 min at 4000×g, the pellets were chilled on ice and then the cells were resuspended in 1/20th of the original culture volume of lysis buffer (1×PBS, 1× Bugbuster® Protein Extraction Reagent (Novagen®), 50 μg/mL DNase 1, 100 μg/mL lysozyme, 1 mM PMSF, and 20 mM imidazole). The cell resuspensions were incubated for 30 min at 4° C., and the lysates were then clarified by centrifugation (16000×g, 4° C., 30 min). The clarified lysate supernatant was then transferred to fresh microcentrifuge tubes containing 50-100 μL of Ni²⁺-NTA slurry (Qiagen). The Ni²⁺-NTA mixture was incubated for 1 h at 4° C. while tumbling gently. The inventors collected the Ni²⁺-NTA beads gravity filtration on a fritted column and resuspended them in 500 μL of wash buffer (PBS, 40 mM imidazole, pH 8); three washes were performed. To elute polyhistidine-tagged proteins, the inventors subsequently resuspended the washed beads in 100 μL of elution buffer (PBS, 300 mM imidazole, pH 8). Following centrifugation (1000×g, 4° C., 1 min), the supernatant with eluted proteins was transferred into fresh microcentrifuge tubes, and the elution procedure repeated three times. To quantify protein expression yields (mg/L), the inventors used the Qubit™ Protein Assay kit (ThermoScientific) according to the manufacturer's protocol and stored purified proteins at −20° C. prior to subsequent analyses.

Purification of GST-Tagged Reporter Proteins from E. coli

Cells were prepared in 10 mL volumes in an identical manner as to the His₆-tagged reporter expressions, except the lysates were incubated with 100 μL of Glutathione Sepharaose™ 4B beads (GE Healthcare) according to the manufacturers' protocol. Following 1 h incubation at 4° C. with gentle agitation, the inventors washed the beads three times with 1 mL of PBS. The inventors then eluted GST-tagged proteins bound to the beads with 150 μL of 10 mM reduced glutathione in 50 mM Tris-HCl pH 8.0. Eluted proteins were mixed to a final concentration of 1×LDS sample buffer (BioRad) and 10 μL of each sample was then loaded into a 4-12% Bis-Tris NuPAGE gel. Following electrophoresis, the gel was stained with InstantBlue (Expedeon) for 30 min followed by a rinse with water.

### Construction of Vectors for Expressing His₆-SUMO-ncPeptide-GyrA

Recoded wild-type GyrA-CBD and His₆-SUMO were ordered as gBlocks (IDT). The plasmids were assembled in a 4-piece assembly with HiFi DNA Assembly Mix (NEB), where the overlapping fragments contained a GS4 linker between SUMO and GyrA. The assembly contained i) PCR amplified GS₄-GyrA-CBD; ii) His₆-SUMO-GS₄; iii) a DNA fragment containing the MmPylRS/MmtRNA^{Pyl}_{CGA}) pair; and iv) the pBAD backbone amplified by PCR from pBAD_Ub-wt_rec. This generated plasmid pBAD_SUMO-GS₄-GyrACBD_MmPylRS_MmtRNA-Pyl-opt(CGA). Variants of this plasmid where the noncanonical peptide encoding sequence and/or a cysteine codon is embedded between SUMO and GyrA, were generated by site-directed mutagenesis. See Data file S2 for sequence details of all plasmids.

### Expression of His6-SUMO-ncPeptide-GyrA-CBD Fusion Protein

The inventors transformed 60 μL of electrocompetent Syn61Δ3(ev5) cells containing the pMB1_MmPylRS_1R26PylRS(CbzK)_MmtRNA-Pyl-opt(CGA)_AlvtRNA-ΔNPyl(8)(CUA) plasmid with a pBAD_SUMO-XXX-GyrA-CBD_MmPylRS_MmtRNA-Pyl-opt(CGA) or pBAD_SUMO-Cys-XXX-GyrA-CBD_MmPylRS_MmtRNA-Pyl-opt(CGA) derivative (XXX denotes the coding sequence for the target peptide). The electroporated cells were recovered in 1 mL SOB at 37° C. while shaking with 200 rpm, after 2 h the cells were diluted in 20 mL 2×YT containing 50 μg/mL kanamycin and 50 μg/mL apramycin. The expression culture was inoculated by addition of the grown preculture into 500 mL TB medium, maintaining the antibiotic selection. The TB culture was grown at 37° C. while being agitated (200 rpm) to an OD₆₀₀ of 2.5-3.0 (˜12 h). Expression was induced by addition of 0.4% L-arabinose and 2 mM of both ncAAs (AllocK, CbzK) was added. After 14 h the culture was partitioned into 250 mL aliquots and the cells harvested by centrifugation (4000 rpm, 20 min, 4° C.). The cell pellets were washed with PBS and stored at −20° C.

### Purification of His6-SUMO-ncPeptide-GyrA-CBD Fusion Protein and Peptide Excision

The frozen cell aliquots were thawed and resuspended in ice cold MES buffer (20 mM MES pH 6.9, 150 mM NaCl), supplemented with DNAse (0.01 mg/mL) and Lysozyme (0.1 mg/mL). The resuspended cells were passed through a pneumatic homogenizer twice and the lysate cleared by centrifugation (25000 rpm, 25 min). The supernatant was mixed with 1 mL of Ni²⁺-NTA slurry (50% beads in 20% ethanol) and incubated for 1 h at 4° C. under gentle agitation. Using a Poly-Prep column (BioRad) the Ni²⁺-NTA agarose was separated from the supernatant through gravity filtration. The filtrate was washed with 30 mL of MES buffer supplemented with 30 mM imidazole and the protein subsequently eluted in 2 mL of MES buffer containing 200 mM imidazole. The eluate was concentrated to approximately 10 mg/mL and the buffer exchanged to MES buffer. 4 mg of protein (˜100 μg of peptide) was used in each excision reaction, which were prepared in 500 μL MES buffer containing 10 or 200 mM DTT (cyclisation or DTT induced cleavage) and 0.04 mg Ulp1. The reaction was incubated at 37° C. and the reaction progress was monitored by LC-MS, upon completion (18-48 h) the free peptide was purified either by C18 spin column purification (linear AllocK/CbzK tetramer) or hot, acidic methanol extraction (linear AllocK/CbzK hexamer, cyclic AllocK/CbzK tetramer).

For C18 spin column (Pierce™) purifications, a protocol was essentially performed as described in the manufacturer's instructions, the only modification being an additional washing step with 70% ACN, 0.1% AcOH to remove proteins and substituting the elution buffer with 90% MeOH, 0.2% AcOH. Briefly, the sample was mixed with 4× sample buffer (20% ACN, 2% AcOH) and loaded in 200 μL portions onto the activated and equilibrated C18 spin column. The reaction was passed twice over the column by centrifugation at 1500×g for 30 s. Then the column was washed twice with 200 μL wash buffer (5% ACN and 0.5% AcOH) and once with 200 μL protein wash buffer (70% ACN, 0.2% AcOH). The peptide was then eluted in 2×20 μL 90% MeOH, 0.2% AcOH, and the methanol was evaporated under an air stream (not to dryness). The sample was then refilled to the initial volume with 0.2% AcOH and analyzed by ESI-MS.

For hot, acidic methanol extractions, the linear AllocK/CbzK hexamer and cyclic AllocK/CbzK tetramer peptides precipitate from the reaction mixture and can be collected by centrifugation (5 min, 20000 rpm), the supernatant was discarded. The peptides were resolubilized in 200 μL of 90% methanol 1% AcOH for 2 h at 55° C. (prolonged incubation increases the loss of Alloc and Cbz protection groups). The samples were reduced to 40 μL by evaporation under an air stream; at this point the methanol concentration was maintained above >20% to ensure solubility. The samples were analysed as described above for the linear AllocK/CbzK tetramer peptide.

### Intact Protein Mass Spectrometry

The inventors carried out ESI-MS using a Waters Xevo G2 mass spectrometer equipped with a modified nanoAcquity LC system. Briefly, injected proteins were separated on a BEH C4 UPLC column (1.7 μm; 1.0×100 mm; Waters) with a flowrate of 50 μL/min. The pump delivered an acetonitrile gradient starting at 2% vol/vol to 80% vol/vol (0.1% vol/vol formic acid) over 20 minutes. The column outlet was directly interfaced via an electrospray ionization source with a hybrid quadrupole time-of-flight mass spectrometer (Waters). While using a cone voltage of 30 V, data were acquired in positive ion mode with a range from 300-2000 m z. Scans were deconvoluted using the MaxEnt1 function within MassLynx software (Waters). The inventors calculated theoretical wild-type protein molecular weights with GPMAW (Lighthouse Data) software and edited them manually to accommodate the molecular weights of ncAAs.

Tandem Mass Spectrometry (MS/MS) of ncAA-Containing Tryptic Peptides

The inventors purified ubiquitin or GFP proteins containing ncAAs by virtue of their C-terminal His₆ tag, as described above, and ran the purified samples on 4-12% NuPAGE Bis-Tris gels (Invitrogen). Following InstantBlue (Expedeon) staining, the inventors excised gel pieces migrating at the approximate molecular weight of the predicted target protein and subjected them to in-gel trypsin digestion as described previously(37)). Excised protein gel pieces were destained with 50% v/v acetonitrile with 50 mM ammonium bicarbonate. After reduction with 10 mM DTT and alkylation with 55 mM iodoacetamide, the proteins were digested overnight at 37° C. with 6 ng/μL of trypsin (Promega, UK). Tryptic peptides were extracted in 2% v/v formic acid with 2% v/v acetonitrile and subsequently analyzed by nano-scale capillary LC-MS/MS with an Ultimate U3000 HPLC (ThermoScientific Dionex, San Jose, USA) set to a flowrate of 300 nL/min. Peptides were trapped on a C18 Acclaim PepMap100 5 μm, 100 μm×20 mm nanoViper (ThermoScientific Dionex, San Jose, USA) prior to separation on a C18 T3 1.8 μm, 75 μm×250 mm nanoEase column (Waters, Manchester, UK). A gradient of acetonitrile eluted the peptides, and the analytical column outlet was directly interfaced using a nano-flow electrospray ionization source, with a quadrupole Orbitrap mass spectrometer (Q-Exactive HFX, ThermoScientific, USA). For data-dependent analysis a resolution of 60,000 for the full MS spectrum was used, followed by twelve MS/MS. MS spectra were collected over a m/z range of 300-1,800. The resultant LC-MS/MS spectra were searched against a protein database (UniProt KB) using the Mascot search engine program. The database search parameters were restricted to a precursor ion tolerance of 5 p.p.m with a fragmented ion tolerance of 0.1 Da. The inventors set multiple modifications in the search parameters: two missed enzyme cleavages, variable modifications for methionine oxidation, cysteine carbamidomethylation, pyroglutamic acid, lysine to ncAA, threonine to ncAA, and serine to ncAA (ncAA variable based upon experiment). The proteomics software Scaffold 4 was used to visualize the fragmented spectra.

### Example 4—Discussion

The inventors have evolved Syn61 and deleted the tRNAs and release factor that decode TCG, TCA and TAG codons. They show that the resulting strain provides complete resistance to a cocktail of viruses. Moreover, they demonstrate the encoded incorporation of non-canonical amino acids (ncAAs) in response to all three codons and the encoded, programmable cellular synthesis of entirely non-canonical heteropolymers and macrocycles.

Thus, the inventors have synthetically uncoupled their strain from the ability to read the canonical code, and this advance provides a potential basis for bioproduction without the catastrophic risks associated with viral contamination and lysis (26, 27). The inventors note that the synthetic codon compression and codon reassignment strategy that they have implemented is analogous to models proposed for codon capture in the course of natural evolution (28).

Future work will expand the principles exemplified herein to further compress and reassign the genetic code. The inventors anticipate that—in combination with ongoing advances in engineering the translational machinery of cells (4)—this work will enable the programmable and encoded cellular synthesis of an expanded set of non-canonical heteropolymers with emergent, and potentially useful, properties.

### REFERENCES

1. F. H. C. Crick, L. Barnett, S. Brenner, R. J. Watts-Tobin, General Nature of the Genetic Code for Proteins. Nature 192, 1227-1232 (1961).
2. P. Marliere, The farther, the safer: a manifesto for securely navigating synthetic species away from the old living world. Systems and Synthetic Biology 3, 77 (2009).
3. J. W. Chin, Expanding and reprogramming the genetic code. Nature 550, 53-60 (2017).
4. D. de la Torre, J. W. Chin, Reprogramming the genetic code. Nature Reviews Genetics 22, 169-184 (2020).
5. T. Passioura, H. Suga, Reprogramming the genetic code in vitro. Trends Biochem Sci 39, 400-408 (2014).
6. A. C. Forster et al., Programming peptidomimetic syntheses by translating genetic codes designed de novo. Proc Natl Acad Sci USA 100, 6353-6357 (2003).
7. M. J. Lajoie et al., Genomically Recoded Organisms Expand Biological Functions. Science 342, 357-360 (2013).
8. N. J. Ma, F. J. Isaacs, Genomic Recoding Broadly Obstructs the Propagation of Horizontally Transferred Genetic Elements. Cell Systems 3, 199-207 (2016).
9. G. Korkmaz, M. Holm, T. Wiens, S. Sanyal, Comprehensive analysis of stop codon usage in bacteria and its correlation with release factor abundance. J Biol Chem 289, 30334-30342 (2014).
10. D. D. Young, P. G. Schultz, Playing with the Molecules of Life. ACS Chemical Biology 13, 854-870 (2018).
11. C. C. Liu, P. G. Schultz, Adding New Chemistries to the Genetic Code. Annual Review of Biochemistry 79, 413-444 (2010).
12. Y. Zhang et al., A semi-synthetic organism that stores and retrieves increased genetic information. Nature 551, 644-647 (2017).
13. E. C. Fischer et al., New codons for efficient production of unnatural proteins in a semisynthetic organism. Nature Chemical Biology 16, 570-576 (2020).
14. H. Neumann, K. Wang, L. Davis, M. Garcia-Alai, J. W. Chin, Encoding multiple unnatural amino acids via evolution of a quadruplet-decoding ribosome. Nature 464, 441-444 (2010).
15. K. Wang et al., Optimized orthogonal translation of unnatural amino acids enables spontaneous protein double-labelling and FRET. Nature Chemistry 6, 393-403 (2014).
16. D. L. Dunkelmann, J. C. W. Willis, A. T. Beattie, J. W. Chin, Engineered triply orthogonal pyrrolysyl-tRNA synthetase/tRNA pairs enable the genetic encoding of three distinct non-canonical amino acids. Nature Chemistry 12, 535-544 (2020).
17. J. S. Italia et al., Mutually Orthogonal Nonsense-Suppression Systems and Conjugation Chemistries for Precise Protein Labeling at up to Three Distinct Sites. Journal of the American Chemical Society 141, 6204-6212 (2019).
18. J. Fredens et al., Total synthesis of Escherichia coli with a recoded genome. Nature 569, 514-518 (2019).
19. W. H. Schmied et al., Controlling orthogonal ribosome subunit interactions enables evolution of new function. Nature 564, 444-448 (2018).
20. M. R. Hemm et al., Small Stress Response Proteins in Escherichia coli: Proteins Missed by Classical Proteomic Studies. Journal of Bacteriology 192, 46 (2010).
21. S. Meydan et al., Retapamulin-Assisted Ribosome Profiling Reveals the Alternative Bacterial Proteome. Mol Cell 74, 481-493 e486 (2019).
22. A. Katz, S. Elgamal, A. Rajkovic, M. Ibba, Non-canonical roles of tRNAs and tRNA mimics in bacterial cell biology. Mol Microbiol 101, 545-558 (2016).
23. Z. Su, B. Wilson, P. Kumar, A. Dutta, Noncanonical Roles of tRNAs: tRNA Fragments and Beyond. Annu Rev Genet 54, 47-69 (2020).
24. L. You, P. F. Suthers, J. Yin, Effects of Escherichia coli Physiology on Growth of Phage T7 In Vivo and In Silico. Journal of Bacteriology 184, 1888 (2002).
25. T. Yanagisawa et al., Multistep Engineering of Pyrrolysyl-tRNA Synthetase to Genetically Encode Ne-(o-Azidobenzyloxycarbonyl) lysine for Site-Specific Protein Modification. Chemistry & Biology 15, 1187-1197 (2008).
26. V. Bethencourt, Virus stalls Genzyme plant. Nature Biotechnology 27, 681-681 (2009).
27. J. Zahn, M. Halter, in Surveillance and Elimination of Bacteriophage Contamination in an Industrial Fermentation Process. (2018).
28. S. Osawa, T. H. Jukes, Codon reassignment (codon capture) in evolution. Journal of Molecular Evolution 28, 271-278 (1989).
29. D. Cervettini, K. C. Liu, J. W. Chin, Scripts for Sense Codon Reassignment Enables Viral Resistance and Encoded Polymer Synthesis. Zenodo, (2021).
30. D. Cervettini et al., Rapid discovery and evolution of orthogonal aminoacyl-tRNA synthetase-tRNA pairs. Nature Biotechnology 38, 989-999 (2020).
31. A. H. Badran, D. R. Liu, Development of potent in vivo mutagenesis plasmids with broad mutational spectra. Nature Communications 6, 8425 (2015).
32. K. Wang et al., Defining synonymous codon compression schemes by genome recoding. Nature 539, 59-64 (2016).
33. D. E. Deatherage, J. E. Barrick, Identification of mutations in laboratory-evolved microbes from next-generation sequencing data using breseq. Methods Mol Biol 1151, 165-188 (2014).
34. H. Thorvaldsdóttir, J. T. Robinson, J. P. Mesirov, Integrative Genomics Viewer (IGV): high-performance genomics data visualization and exploration. Briefings in Bioinformatics 14, 178-192 (2013).
35. P. J. A. Cock et al., Biopython: freely available Python tools for computational molecular biology and bioinformatics. Bioinformatics 25, 1422-1423 (2009).
36. J. D. Hunter, Matplotlib: A 2D Graphics Environment. Computing in Science & Engineering 9, 90-95 (2007).
37. T. S. Elliott et al., Proteome labeling and protein identification in specific tissues and at specific developmental stages in an animal. Nature Biotechnology 32, 465-472 (2014).
38. C. Fan, H. Xiong, N. M. Reynolds, D. Söll, Rationally evolving tRNAPyl for efficient incorporation of noncanonical amino acids. Nucleic Acids Research 43, e156-e156 (2015).
39. J. N. Beyer et al., Overcoming Near-Cognate Suppression in a Release Factor 1-Deficient Host with an Improved Nitro-Tyrosine tRNA Synthetase. Journal of Molecular Biology 432, 4690-4704 (2020).
40. K. C. Schultz et al., A Genetically Encoded Infrared Probe. Journal of the American Chemical Society 128, 13984-13985 (2006).
41. A. Telenti et al., The Mycobacterium xenopi GyrA protein splicing element: characterization of a minimal intein. Journal of Bacteriology 179, 6378 (1997).

Data File S1, Data File S2, and Data File S3 as referred to herein are found in the publication: Wesley E. Robertson, Louise F. H. Funke, Daniel de la Torre, Julius Fredens, Thomas S. Elliott, Martin Spinck, Yonka Christova, Daniele Cervettini, Franz L. Boge, Kim C. Liu, Salvador Buse, Sarah Maslen, George P. C. Salmond, & Jason W. Chin, Sense Codon Reassignment Enables Viral Resistance and Encoded Polymer Synthesis, Science, and are incorporated by reference in their entirety.

1. A prokaryotic cell, wherein:
the prokaryotic cell does not express a first endogenous tRNA and a second endogenous tRNA; and
the prokaryotic cell comprises a genome wherein a first type of sense codon and a second type of sense codon have been recoded such that the first endogenous tRNA and the second endogenous tRNA are dispensable.

2. The prokaryotic cell of claim 1, wherein:
the essential genes of the genome do not contain occurrences of the first type of sense codon, and the first endogenous tRNA is a cognate tRNA for the first type of sense codon; and/or
the essential genes of the genome do not contain occurrences of the second type of sense codon, and the second endogenous tRNA is a cognate tRNA for the second type of sense codon.

3. The prokaryotic cell of claim 1 or claim 2, wherein:
the genome comprises 5, 4, 3, 2, 1, or no occurrences of the first type of sense codon, and the first endogenous tRNA is a cognate tRNA for the first type of sense codon; and/or
the genome of comprises 5, 4, 3, 2, 1, or no occurrences of the second type of sense codon, and the second endogenous tRNA is a cognate tRNA for the second type of sense codon.

4. The prokaryotic cell of any one of claims 1 to 3, wherein
the first type of sense codon is TCA and the first endogenous tRNA is tRNA^{Ser}_{UGA}; and/or
the second type of sense codon is TCG and the second endogenous is tRNA^{Ser}_{CGA}.

5. The prokaryotic cell of claim 4, wherein
a plurality of occurrences of the TCA codon in the parental strain have been replaced with AGT; and/or
a plurality of occurrences of the TCG codon in the parental strain have been replaced with AGC.

6. The prokaryotic cell of any one of claims 1 to 5, wherein:
the prokaryotic cell does not express a first endogenous release factor; and
a first type of stop codon has been recoded within the genome such that the first endogenous release factor is dispensable.

7. The prokaryotic cell of claim 6, wherein the essential genes of the genome do not contain occurrences of the first type of stop codon, and the first endogenous release factor is a cognate release factor for the first type of stop codon.

8. The prokaryotic cell of claim 6 or claim 7, wherein the genome comprises 5, 4, 3, 2, 1, or no occurrences of the first type of stop codon, and the first endogenous release factor is a cognate release factor for the first type of stop codon.

9. The prokaryotic cell of any one of claims 6 to 8, wherein the first type of stop codon is TAG and wherein the first endogenous release factor is RF-1.

10. The prokaryotic cell of claim 9, wherein occurrences of the TAG codon in the parental strain have been replaced with TAA.

11. A prokaryotic cell comprising a genome that is at least 80%, 85%, 90%, 95%, 98%, 99%, 99.5%, 99.8%, 99.9%, or 100% identical to any one of SEQ ID NOs: 3 to 8.

12. The prokaryotic cell of any one of claims 6 to 11, wherein
the prokaryotic cell expresses a third orthogonal aminoacyl-tRNA synthetase and a third orthogonal tRNA,
the third orthogonal aminoacyl-tRNA synthetase and the third orthogonal tRNA form a third orthogonal aminoacyl-tRNA synthetase-tRNA pair, and
the third orthogonal tRNA is capable of decoding the first type of stop codon.

13. The prokaryotic cell of claim 12, wherein the third orthogonal aminoacyl-tRNA synthetase-tRNA pair is:
AfTyrRS or a variant with altered selectivity to a non-canonical amino acid, and Af-tRNA^{Tyr(A01)}_{YYY}; or
MjTyrRS or a variant with altered selectivity to a non-canonical amino acid, and MjtRNA^{Tyr}_{YYY}.

14. The prokaryotic cell of any one of claims 1 to 13, wherein
the prokaryotic cell expresses a first orthogonal aminoacyl-tRNA synthetase and a first orthogonal tRNA,
the first orthogonal aminoacyl-tRNA synthetase and the first orthogonal tRNA form a first orthogonal aminoacyl-tRNA synthetase-tRNA pair, and
the first orthogonal tRNA is capable of decoding the first type of sense codon.

15. The prokaryotic cell of claim 14, wherein the first orthogonal aminoacyl-tRNA synthetase is MmPylRS or a variant with altered selectivity to a non-canonical amino acid, and the first orthogonal tRNA is MmtRNA^{Pyl}_{YYY} or MmtRNA^{Pyl}_{UGA}.

16. The prokaryotic cell of any one of claims 1 to 15, wherein
the prokaryotic cell expresses a second orthogonal aminoacyl-tRNA synthetase and a second tRNA,
the second orthogonal aminoacyl-tRNA synthetase and the second orthogonal tRNA form a second orthogonal aminoacyl-tRNA synthetase-tRNA pair, and
the second orthogonal tRNA is capable of decoding the second type of sense codon.

17. The prokaryotic cell of claim 16, wherein the second orthogonal aminoacyl-tRNA synthetase is 1R26PylRS or a variant with altered selectivity to a non-canonical amino acid, and the second orthogonal tRNA is AlvtRNA^{ΔNPyl(8)}_{YYY} or AlvtRNA^{ΔNPyl(8)}_{CGA}.

18. The prokaryotic cell of any one of claims 1 to 17, wherein the growth rate of the prokaryotic cell is faster than the growth rate of a reference prokaryotic cell of a parental strain.

19. The prokaryotic cell of claim 18, wherein the reference prokaryotic cell is of a parental strain directly obtained upon recoding of the genome to remove the first type of sense codon, the second type of sense codon, and the first type of stop codon.

20. The prokaryotic cell of claim 18, wherein the reference prokaryotic cell is of a parental strain directly obtained upon removal of the first endogenous tRNA, the second endogenous tRNA, and the first endogenous release factor.

21. The prokaryotic cell of any one of claims 1 to 20, wherein the prokaryotic cell is resistant to phage infection and/or horizontal transfer of the F plasmid.

22. The prokaryotic cell of any one of claims 1 to 20, wherein the prokaryotic cell is completely resistant to phage infection and/or horizontal transfer of the F plasmid.

23. The prokaryotic cell of any one of claims 1 to 22, wherein the prokaryotic cell is a bacterial cell or an Escherichia coli cell.

24. The prokaryotic cell of any one of claims 1 to 23, wherein the prokaryotic cell is viable.

25. A method for producing a modified prokaryotic cell, wherein the method comprises:
(i) modifying a prokaryotic cell to express a first orthogonal aminoacyl-tRNA synthetase-tRNA pair suitable for decoding a first type of sense codon, wherein
the prokaryotic cell comprises a genome wherein the first type of sense codon has been recoded such that a first endogenous tRNA is dispensable;
(ii) incubating the prokaryotic cell in the presence of a non-canonical amino acid which is a substrate for the first orthogonal aminoacyl-tRNA synthetase; and
(iii) modifying the endogenous gene encoding the first endogenous tRNA such that the first endogenous tRNA is not expressed.

26. The method of claim 25, wherein steps (i) and (ii) are performed before step (iii).

27. The method of claim 25 or claim 26, wherein the method further comprises:
(a) modifying the prokaryotic cell to express a second orthogonal aminoacyl-tRNA synthetase-tRNA pair suitable for decoding a second type of sense codon, wherein
the prokaryotic cell comprises a genome wherein the second type of sense codon has been recoded such that a second endogenous tRNA is dispensable;
(b) incubating the prokaryotic cell in the presence of a non-canonical amino acid which is a substrate for the second orthogonal aminoacyl-tRNA synthetase; and
(c) modifying the endogenous gene encoding the second endogenous tRNA such that the second endogenous tRNA is not expressed.

28. The method of claim 27, wherein steps (i) and (ii) are performed before step (iii), and/or wherein steps (a) and (b) are performed before step (c).

29. The method of claim 27 or 28, wherein
steps (a), (b), and (c) are performed before steps (i), (ii), (iii); or
step (a) is performed concurrently with step (i), step (b) is performed concurrently with step (ii), and step (c) is performed concurrently with step (iii); or
steps (a), (b), and (c) are performed after steps (i), (ii), (iii).

30. The method of any one of claims 25 to 29, wherein the essential genes of the genome do not contain occurrences of the first type of sense codon, or the genome comprises 5, 4, 3, 2, 1, or no occurrences of the first type of sense codon.

31. The method of any one of claims 25 to 30, wherein the first type of sense codon is TCA and the first endogenous tRNA is tRNA^{Ser}_{UGA}.

32. The method of claim 31, wherein the TCA codon has been replaced with AGT.

33. The method of any one of claims 25 to 32, wherein the first orthogonal aminoacyl-tRNA synthetase is MmPylRS or a variant with altered selectivity to a non-canonical amino acid, and the first orthogonal tRNA is MmtRNA^{Pyl}_{YYY} or MmtRNA^{Pyl}_{UGA}.

34. The method of any one of claims 25 to 33, wherein the essential genes of the genome do not contain occurrences of the second type of sense codon, or the genome of comprises 5, 4, 3, 2, 1, or no occurrences of the second type of sense codon.

35. The method of any one of claims 25 to 34, wherein the second type of sense codon is TCG and the second endogenous tRNA is tRNA^{Ser}_{CGA}.

36. The method of claim 35, wherein the TCG codon is replaced with AGC.

37. The method of any one of claims 25 to 36, wherein the second orthogonal aminoacyl-tRNA synthetase is 1R26PylRS or a variant with altered selectivity to a non-canonical amino acid, and the second orthogonal tRNA is AlvtRNA^{ΔNPyl(8)}_{YYY} or AlvtRNA^{ΔNPyl(8)}_{CGA}.

38. The method of any one of claims 25 to 37, wherein the genome of the prokaryotic cell to which the method is applied is at least 80%, 85%, 90%, 95%, 98%, 99%, 99.5%, 99.8%, 99.9%, or 100% identical to any one of: GenBank accession number CP040347.1, SEQ ID NO: 3, or SEQ ID NO: 4.

39. The method of any one of claims 25 to 38, further comprising
modifying the prokaryotic cell to express a third orthogonal aminoacyl-tRNA synthetase-tRNA pair suitable for decoding a first type of stop codon, wherein a first type of stop codon has been recoded within the genome such that a first endogenous release factor is dispensable.

40. The method of claim 39, wherein the essential genes of the genome do not contain occurrences of the first type of stop codon or the genome comprises 5, 4, 3, 2, 1, or no occurrences of the first type of stop codon.

41. The method of claim 39 or 40, wherein the first type of stop codon is TAG and wherein the cognate release factor for the first type of stop codon is RF-1.

42. The method of claim 41, wherein the TAG codon is replaced with TAA.

43. The method of any one of claims 39 to 42, wherein the third orthogonal aminoacyl-tRNA synthetase-tRNA pair is:
AfTyrRS or a variant with altered selectivity to a non-canonical amino acid, and Af-tRNA^{Tyr(A01)}_{YYY}; or
MjTyrRS or a variant with altered selectivity to a non-canonical amino acid, and MjtRNA^{Tyr}_{YYY}.

44. The method of any one of claims 25 to 43, comprising, prior to step (i) and (a):
inducing mutagenesis in a cell culture comprising the prokaryotic cell that comprises the recoded genome,
maintaining the cell culture under exponential growth conditions,
selecting a prokaryotic cell, from said cell culture, with an increased growth rate compared to the initial culture; and
wherein step (i) or (a) is applied to the selected prokaryotic cell.

45. The method of claim 44, wherein the mutagenesis, mutation, and selection are part of a parallel mutagenesis and dynamic parallel selection process.

46. The method of claim 44 or claim 45, wherein induction of mutagenesis comprises the use of a mutagenesis plasmid.

47. The method of claim 46, wherein the mutagenesis plasmid is MP6.

48. The method of any one of claims 44 to 47, wherein prior to step (i), two rounds of mutagenesis and selection are applied.

49. The method of any one of claims 25 to 48, further comprising, after step (iii) and/or (c):
obtaining a cell culture from the prokaryotic cell,
inducing mutagenesis in the cell culture,
maintaining the cell culture under exponential growth conditions, and
selecting a prokaryotic cell, from said cell culture, with an increased growth rate compared to the initial culture.

50. The method of claim 49, wherein the mutagenesis, mutation, and selection are part of a parallel mutagenesis and dynamic parallel selection process.

51. The method of claim 49 or claim 50, wherein induction of mutagenesis comprises the use of a mutagenesis plasmid, wherein the mutagenesis plasmid does not contain any occurrences of the first or second type of sense codon nor any occurrences of the first type of stop codon.

52. The method of claim 51, wherein the mutagenesis plasmid is MP6, wherein the MP6 has been recoded to not contain any occurrences of the first or second type of sense codon nor any occurrences of the first type of stop codon.

53. The method of any one of claims 49 to 52, wherein three rounds of mutagenesis and selection are applied.

54. The method of any one of claims 44 to 53, wherein
mutagenesis is carried out for 5, 10, 15, 17, 20, 30, 45, 60, 70, 80, 100, 150, 200, or more generations; and/or
the cell culture is maintained under exponential growth conditions for 5, 10, 15, 20, 30, 40, 50, 52, 60, 70, 80, 100, 200, or more generations.

55. The method of any one of claims 25 to 54, wherein the prokaryotic cell is a bacterial cell or an Escherichia coli cell.

56. A method of synthesising a polymer, comprising:
i) providing a prokaryotic cell of any one of claims 1 to 24, or generated according to the methods of any one of claims 25 to 55, wherein the prokaryotic cell comprises the first orthogonal aminoacyl-tRNA synthetase-tRNA pair; and
contacting said prokaryotic cell with a nucleic acid sequence encoding a polymer, said nucleic acid sequence comprising the first type of sense codon;
ii) incubating the prokaryotic cell in the presence of a first non-canonical amino acid, wherein the first non-canonical amino acid is a substrate for the first orthogonal aminoacyl-tRNA synthetase; and
iii) incubating the prokaryotic cell to allow incorporation of the first non-canonical amino acid into the polymer via the first orthogonal aminoacyl-tRNA synthetase-tRNA pair.

57. The method of claim 56, wherein the prokaryotic cell comprises the second orthogonal aminoacyl-tRNA synthetase-tRNA pair and the nucleic acid sequence comprises the second type of sense codon, and wherein
step ii) comprises incubating the prokaryotic cell in the presence of a second non-canonical amino acid, wherein the second non-canonical amino acid is a substrate for the second orthogonal aminoacyl-tRNA synthetase; and
step iii) comprises incubating the prokaryotic cell to allow incorporation of the second non-canonical amino acid into the polymer via the second first orthogonal aminoacyl-tRNA synthetase-tRNA pair.

58. The method of claim 56 or 57, wherein the prokaryotic cell comprises the third orthogonal aminoacyl-tRNA synthetase-tRNA pair and the nucleic acid sequence comprises the first type of stop codon, and wherein
step ii) comprises incubating the prokaryotic cell in the presence of a third non-canonical amino acid, wherein the third non-canonical amino acid is a substrate for the third orthogonal aminoacyl-tRNA synthetase; and
step iii) comprises incubating the prokaryotic cell to allow incorporation of the third non-canonical amino acid into the polymer via the third first orthogonal aminoacyl-tRNA synthetase-tRNA pair.

59. The method of any one of claims 56 to 58, further comprising:
iv) purifying the synthesised polymer.

60. The method of any one of claims 56 to 59, wherein the synthesised polymer comprises at least one non-canonical amino acid directly adjacent to another non-canonical amino acid.

61. The method of any one of claims 56 to 60, wherein the polymer comprises a chain of two, three, four, five, six, seven, eight, nine, ten, 15, 20, or more non-canonical amino acids directly adjacent to each other.

62. The method of any one of claims 56 to 61, wherein the polymer is a macrocycle.

63. The method of any one of claims 56 to 62, wherein the non-canonical amino acids comprise any one of, or any combination of, BocK, CbzK, AllocK, p-I-Phe, CypK, AlkK, 3-Nitro-Tyr, and p-Az-Phe.

64. A polymer obtained from or obtainable by the methods of any one of claims 56 to 63.

65. An isolated 1R26PylRS, or a variant with altered selectivity to a non-canonical amino acid.

66. The isolated 1R26PylRS of claim 65, wherein the isolated 1R26PylRS is at least 50%, 60%, 70%, 80%, 90%, 95%, 99%, or 100% identical to SEQ ID NO: 9, comprising any one of the following groups of mutations: i) L121L, L125L, Y126G, M129L, N166N, V168V, Y206Y, A223A; ii) L121L, L125L, Y126Y, M129A, N166N, V168V, Y206F, A223A; iii) L121L, L125L, Y126Y, M129M, N166Q, V168V, Y206F, A223A; iv) L121L, L125L, Y126Y, M129M, N166S, V168M, Y206F, A223G; and v) L121M, L125I, Y126F, M129A, N166N, V168F, Y206Y, A223A.

67. The isolated 1R26PylRS of claim 65, wherein the isolated 1R26PylRS is at least 50%, 60%, 70%, 80%, 90%, 95%, 99%, or 100% similar or identical to any one of SEQ ID NOs: 10 to 14.

68. An isolated Archaeoglobus fulgidus tyrosyl-tRNA synthetase (AfTyrRS), or a variant with altered selectivity to a non-canonical amino acid.

69. The isolated AfTyrRS of claim 68, wherein the isolated AfTyrRS is at least 50%, 60%, 70%, 80%, 90%, 95%, 99%, or 100% identical to SEQ ID NO: 16, comprising any one of the following groups of mutations: i) Y36I, L69M, H74L, Q116E, D165T, I166G, and N190N; ii) Y36T, L69L, H74L, Q116E, D165T, I166G, N190K; and iii) Y36I, L69L, H74L, Q116E, D165T, I166G, N190N.

70. The isolated AfTyrRS of claim 68, wherein the isolated AfTyrRS is at least 50%, 60%, 70%, 80%, 90%, 95%, 99%, or 100% similar or identical to any one of SEQ ID NOs: 16 to 19.

71. An isolated Lum1PylRS, or a variant with altered selectivity to a non-canonical amino acid.

72. The isolated Lum1PylRS of claim 71, wherein the Lum1PylRS is at least 50%, 60%, 70%, 80%, 90%, 95%, 99%, or 100% identical to SEQ ID NO: 32, comprising any one of the following groups of mutations: i) L121L, L125L, Y126G, M129L, V168V; and ii) L121M, L125I, Y126F, M129A, V168F.

73. The isolated Lum1PylRS of claim 71, wherein the Lum1PylRS is at least 50%, 60%, 70%, 80%, 90%, 95%, 99%, or 100% similar or identical to any one of SEQ ID NOs: 32 to 34.

74. An isolated NitroPylRS, or a variant with altered selectivity to a non-canonical amino acid.

75. The isolated NitroPylRS of claim 74, wherein the NitroPylRS is at least 50%, 60%, 70%, 80%, 90%, 95%, 99%, or 100% identical to SEQ ID NO: 36, comprising the following substitutions L123M, L127I, Y128F, M131A, and V169F.

76. The isolated NitroPylRS of claim 74, wherein the NitroPylRS is at least 50%, 60%, 70%, 80%, 90%, 95%, 99%, or 100% similar or identical to SEQ ID NO: 35 or SEQ ID NO: 36.

77. An isolated ClosΔNTDPylRS, or a variant with altered selectivity to a non-canonical amino acid.

78. The isolated ClosΔNTDPylRS of claim 77, wherein the ClosΔNTDPylRS is at least 50%, 60%, 70%, 80%, 90%, 95%, 99%, or 100% identical to SEQ ID NO: 37, comprising any one of the following groups of mutations: i) Y126G, M129L, Y208Y, and ii) Y126Y, M129A, Y208F.

79. The isolated ClosΔNTDPylRS of claim 77, wherein the ClosΔNTDPylRS is at least 50%, 60%, 70%, 80%, 90%, 95%, 99%, or 100% similar or identical to any one of SEQ ID NOs: 37 to 39.

80. An isolated TronΔNTDPylRS, or a variant with altered selectivity to a non-canonical amino acid.

81. The isolated TronΔNTDPylRS of claim 80, wherein the TronΔNTDPylRS is at least 50%, 60%, 70%, 80%, 90%, 95%, 99%, or 100% similar or identical to SEQ ID NO: 40.

82. An isolated GemmΔNTDPylRS, or a variant with altered selectivity to a non-canonical amino acid.

83. The isolated GemmΔNTDPylRS of claim 82, wherein the GemmΔNTDPylRS is at least 50%, 60%, 70%, 80%, 90%, 95%, 99%, or 100% similar or identical to SEQ ID NO: 41.

84. An isolated PGA8ΔNTDPylRS, or a variant with altered selectivity to a non-canonical amino acid.

85. The isolated PGA8ΔNTDPylRS of claim 84, wherein the PGA8ΔNTDPylRS is at least 50%, 60%, 70%, 80%, 90%, 95%, 99%, or 100% similar or identical to SEQ ID NO: 42.

86. An isolated I2ΔNTDPylRS, or a variant with altered selectivity to a non-canonical amino acid.

87. The isolated I2ΔNTDPylRS of claim 86, wherein the I2ΔNTDPylRS is at least 50%, 60%, 70%, 80%, 90%, 95%, 99%, or 100% similar or identical to SEQ ID NO: 43.

88. An isolated D121ΔNTDPylRS, or a variant with altered selectivity to a non-canonical amino acid.

89. The isolated D121ΔNTDPylRS of claim 88, wherein the D121ΔNTDPylRS is at least 50%, 60%, 70%, 80%, 90%, 95%, 99%, or 100% similar or identical to SEQ ID NO: 44.

90. An isolated D416ΔNTDPylRS, or a variant with altered selectivity to a non-canonical amino acid.

91. The isolated D416ΔNTDPylRS of claim 90, wherein the D416ΔNTDPylRS is at least 50%, 60%, 70%, 80%, 90%, 95%, 99%, or 100% similar or identical to SEQ ID NO: 45.

92. The tRNA synthetase according to any one of claims 65 to 91, wherein the tRNA synthetase has altered selectivity towards any of: AllocK, AlkK, BocK, Bta, CbzK, CypK, 3-Nitro-Tyr, NMH, p-I-Phe, and p-Az-Phe, and o-Methyl-Tyrosine.

93. An isolated tRNA, wherein the isolated tRNA is at least 80%, 90%, 95%, 99%, or 100% identical to any one of SEQ ID NOs: 15, 20, 46-52, 54-58, 61-66.

94. A host cell comprising the tRNA synthetase according to any one of claims 65 to 92 and a tRNA, wherein the tRNA synthetase and tRNA form an orthogonal aminoacyl-tRNA synthetase-tRNA pair.

95. A host cell comprising any combination of two or three items selected from the list:
(i) the tRNA synthetase according to any one of claims 65 to 67 and a tRNA, wherein the tRNA synthetase and tRNA form an orthogonal aminoacyl-tRNA synthetase-tRNA pair;
(ii) an Archaeoglobus fulgidus tyrosyl-tRNA synthetase (AfTyrRS), or a variant with altered selectivity to a non-canonical amino acid, and an Af-tRNA^{Tyr(A01)}_{YYY} wherein the AfTyrRS and Af-tRNA^{Tyr(A01)}_{YYY} form an orthogonal aminoacyl-tRNA synthetase-tRNA pair;
(iii) a MmPylRS tRNA synthetase, or a variant with altered selectivity to a non-canonical amino acid, and a MmtRNA^{Pyl}_{YYY}, wherein the MmPylRS tRNA synthetase and the MmtRNA^{Pyl}_{YYY} form an orthogonal aminoacyl-tRNA synthetase-tRNA pair.

96. A host cell comprising any combination of two or three items selected from the list:
(i) the tRNA synthetase according to any one of claims 65 to 67 and a tRNA, wherein the tRNA synthetase and tRNA form an orthogonal aminoacyl-tRNA synthetase-tRNA pair;
(ii) an AfTyrRS, or a variant with altered selectivity to a non-canonical amino acid, and an Af-tRNA^{Tyr(A01)}_{YYY}, wherein the AfTyrRS and Af-tRNA^{Tyr(A01)}_{YYY} form an orthogonal aminoacyl-tRNA synthetase-tRNA pair;
(iii) an M jannaschii tyrosyl-tRNA synthetase (MjTyrRS), or a variant with altered selectivity to a non-canonical amino acid, and a MjtRNA^{Tyr}_{YYY}, wherein the MjTyrRS and the MjtRNA^{Tyr}_{YYY} form an orthogonal aminoacyl-tRNA synthetase-tRNA pair.

97. A host cell comprising any combination of two or three items selected from the list:
i) a class A ΔNPylRS/^{ΔNPyl}tRNA pair;
ii) a class B ΔNPylRS/^{ΔNPyl}tRNA pair; and
iii) an MmPylRS/Spe^{Pyl}tRNA pair.

98. The host cell of any one of claims 94 to 96, wherein the host cell is a prokaryotic cell or eukaryotic cell, a bacterial cell, an E. coli. cell, a mammalian cell, an insect cell, or a human cell.

99. A method for improving the growth rate of a prokaryotic cell wherein the genome has been recoded, wherein the method comprises:
inducing mutagenesis in a cell culture comprising the prokaryotic cell that comprises the recoded genome,
maintaining the cell culture under exponential growth conditions,
selecting a prokaryotic cell, from said cell culture, with an increased growth rate compared to the initial culture.

100. The method of claim 99, wherein the mutagenesis, mutation, and selection are part of a parallel mutagenesis and dynamic parallel selection process.

101. The method of claim 99 or claim 100, wherein induction of mutagenesis comprises the use of a mutagenesis plasmid, optionally wherein the mutagenesis plasmid is a recoded mutagenesis plasmid.

102. The method of claim 101, wherein the mutagenesis plasmid is MP6.

103. The method of any one of claims 99 to 102, wherein two rounds of mutagenesis and selection are applied.

104. The method of any one of claims 99 to 103, wherein
mutagenesis is carried out for 5, 10, 15, 17, 20, 30, 45, 60, 70, 80, 100, 150, 200, or more generations; and/or
the cell culture is maintained under exponential growth conditions for 5, 10, 15, 20, 30, 40, 50, 52, 60, 70, 80, 100, 200, or more generations.

105. The method of any one of claims 99 to 104 wherein the prokaryotic cell is a bacterial cell or an Escherichia coli cell.

106. A prokaryotic cell obtained or obtainable by the method of any one of claims 99 to 105, wherein the growth rate of the prokaryotic cell is faster than the growth rate of a reference prokaryotic cell of a parental strain.

107. A prokaryotic cell, wherein the prokaryotic cell:
does not express a first endogenous tRNA;
comprises a genome wherein a first type of sense codon has been recoded such that the first endogenous tRNA is dispensable;
expresses a first orthogonal aminoacyl-tRNA synthetase and a first orthogonal tRNA,
the first orthogonal aminoacyl-tRNA synthetase and the first orthogonal tRNA form a first orthogonal aminoacyl-tRNA synthetase-tRNA pair, and
the first orthogonal tRNA is capable of decoding the first type of sense codon.

108. The prokaryotic cell of claim 107, wherein:
the essential genes of the genome do not contain occurrences of the first type of sense codon, and the first endogenous tRNA is a cognate tRNA for the first type of sense codon.

109. The prokaryotic cell of claim 107 or claim 108, wherein:
the genome comprises 5, 4, 3, 2, 1, or no occurrences of the first type of sense codon, and the first endogenous tRNA is a cognate tRNA for the first type of sense codon.

110. The prokaryotic cell of any one of claims 107 to 109, wherein
the first type of sense codon is TCA and the first endogenous tRNA is tRNA^{Ser}_{UGA}; or
the first type of sense codon is TCG and the first endogenous is tRNA^{Ser}_{CGA}

111. The prokaryotic cell of claim 110, wherein
a plurality of occurrences of the TCA codon in the parental strain have been replaced with AGT; and/or
a plurality of occurrences of the TCG codon in the parental strain have been replaced with AGC.

112. The prokaryotic cell of any one of claims 107 to 111, wherein the prokaryotic cell:
does not express a second endogenous tRNA;
comprises a genome wherein a second type of sense codon has been recoded such that the second endogenous tRNA is dispensable;
expresses a second orthogonal aminoacyl-tRNA synthetase and a second tRNA,
the second orthogonal aminoacyl-tRNA synthetase and the second orthogonal tRNA form a second orthogonal aminoacyl-tRNA synthetase-tRNA pair, and
the second orthogonal tRNA is capable of decoding the second type of sense codon.

113. The prokaryotic cell of claim 112, wherein:
the essential genes of the genome do not contain occurrences of the second type of sense codon, and the second endogenous tRNA is a cognate tRNA for the second type of sense codon.

114. The prokaryotic cell of claim 112 or claim 113, wherein:
the genome of comprises 5, 4, 3, 2, 1, or no occurrences of the second type of sense codon, and the second endogenous tRNA is a cognate tRNA for the second type of sense codon.

115. The prokaryotic cell of any one of claims 112 to 114, wherein
the second type of sense codon is TCA and the second endogenous tRNA is tRNA^{Ser}_{UGA}; or
the second type of sense codon is TCG and the second endogenous is tRNA^{Ser}_{CGA}.

116. The prokaryotic cell of any one of claims 107 to 115, wherein:
the prokaryotic cell does not express a first endogenous release factor;
a first type of stop codon has been recoded within the genome such that the first endogenous release factor is dispensable;
the prokaryotic cell expresses a third orthogonal aminoacyl-tRNA synthetase and a third orthogonal tRNA,
the third orthogonal aminoacyl-tRNA synthetase and the third orthogonal tRNA form a third orthogonal aminoacyl-tRNA synthetase-tRNA pair, and
the third orthogonal tRNA is capable of decoding the first type of stop codon.

117. The prokaryotic cell of claim 116, wherein the essential genes of the genome do not contain occurrences of the first type of stop codon, and the first endogenous release factor is a cognate release factor for the first type of stop codon.

118. The prokaryotic cell of claim 116 or 117, wherein the genome comprises 5, 4, 3, 2, 1, or no occurrences of the first type of stop codon, and the first endogenous release factor is a cognate release factor for the first type of stop codon.

119. The prokaryotic cell of any one of claims 116 to 118, wherein the first type of stop codon is TAG and wherein the first endogenous release factor is RF-1.

120. The prokaryotic cell of claim 119, wherein occurrences of the TAG codon in the parental strain have been replaced with TAA.

121. The prokaryotic cell of any one of claims 107 to 120, wherein the prokaryotic cell is a bacterial cell or an Escherichia coli cell.

122. The prokaryotic cell of any one of claims 107 to 121, wherein the prokaryotic cell is viable.
